(19) United States
(12) Patent Application Publication
(10) Pub. No.: US 2007/0213339 A1
(43) Pub. Date: Sep. 13, 2007

(54) Quinolone Carboxylic Acid Derivatives for Treatment of Hyperproliferative Conditions

(75) Inventors: Uday Khire, Orange, CT (US); Xiao-Gao Liu, New Haven, CT (US); Dhanapalan Nagarathnam, Bethany, CT (US); Jill Wood, North Haven, CT (US); Lei Wang, New Haven, CT (US); Donglei Liu, West Haven, CT (US); Jin Zhao, Middletown, CT (US); Leatte Guernon, Hamden, CT (US); Lei Zhang, Hamden, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 10/594,978

(22) PCT Filed: Mar. 31, 2005
(86) PCT No.: PCT/US05/10999

§ 371 Date: Sep. 29, 2006

Related U.S. Application Data
(60) Provisional application No. 60/558,432, filed on Mar. 31, 2004.

(51) Int. Cl. A61K 31/506; A61K 31/496; C07D 403/14

(52) U.S. Cl. 514/252.18; 514/253.08; 544/295; 544/363

(57) ABSTRACT

Quinolone carboxylic acid derivatives of formula (I) 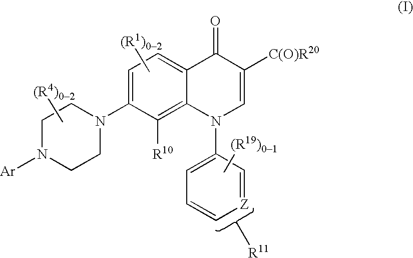
wherein Ar is an optionally substituted phenyl, pyridyl, or pyrimidinyl group and the substituent groups R¹, R⁴, R¹⁰, R¹¹, R¹⁹, and R²⁰ are as defined in the specification, pharmaceutical compositions containing them, and methods of using them in treatment of hyperproliferative diseases such as cancer are disclosed and claimed.

### FIELD

This invention relates to certain quinolone carboxylic acid derivatives and their use for preventing or treating hyper-proliferative disorders.

### BACKGROUND

Quinolone derivatives are known to possess antibacterial and antiviral properties. See, for example WO96/0251-0 (Bayer AG); Filipponi et al., J. Computer-Aided Mol. Design, 15, 203-217 (2001); WO96/02511 (Bayer AG); WO96/02533 (Bayer AG); WO96/02532 (Bayer AG); EP 612731 (Bayer AG); WO01/36408; U.S. Pat. No. 5,639,886 (Bayer AG); EP 0531958 (Mediolanum Farmaceutici); WO 02/059116 (Pharmacia & Upjohn); WO 03/002560 (Vita-Invest); WO 03/032962 (Morphochem AG); WO 03/031443 (Morphochem AG); WO 03/03 1441 (Morphochem AG); WO 99/42106 (Sankyo).

Some quinolone derivatives have also been recognized as having antitumor properties. See, for example Tomita, et al., J. Med. Chem., 45, 5564-5575 (2002).

The art is always desirous of new antitumor agents. New quinolone derivatives having antitumor properties are the subject of the present invention.

### BRIEF SUMMARY

In one embodiment, the present invention relates to compounds having the structural formula (I)

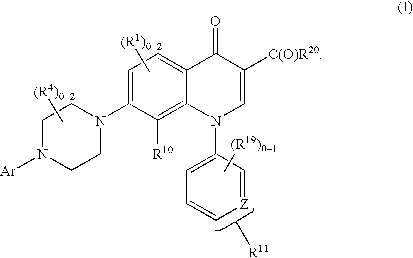

In this formula, the various groups are broadly defined as follows:

R¹ represents —F, —Cl, —Br, —NO₂, —(C₁₋₃ alkyl) optionally substituted with halogen, or —NR²R³, wherein R² and R¹ are independently H or C₁₋₃-alkyl which is optionally substituted with halogen.

R⁴ represents —F, —Cl, —Br, or —(C₁₋₃ alkyl) optionally substituted with halogen.

Ar represents

a1) 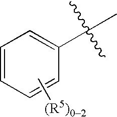
wherein R⁵ represents —F, —Cl, —Br, —(C₁₋₃ alkyl) optionally substituted with halogen, —O(C₁₋₃ alkyl) optionally substituted with halogen, —S(C₁₋₃ alkyl) optionally substituted with halogen, —CN, —C(O)NH₂, —SO₂NH₂, —C(O)CH₃, —NO₂, or —NR⁶R⁷ in which R⁶ and R⁷ are independently H or —(C₁₋₃ alkyl) optionally substituted with halogen;

a2) 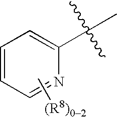
wherein R⁸ represents —CN, —(C₁₋₃ alkyl) optionally substituted with halogen, —O(C₁₋₃ alkyl) optionally substituted with halogen, —C(O)NH₂, or —SO₂NH₂; or

a3) 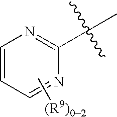
wherein R⁹ represents —F, —Cl, or —Br.

R¹⁰ represents —Cl, —Br, —(C₁₋₃ alkyl) optionally substituted with halogen, —O(C₁₋₃-alkyl) optionally substituted with halogen, or —CN.

Z represents C or N.

When Z is C, R¹¹ is located on one of the two ring atoms encompassed by the bracket and the other of the two ring atoms encompassed by the bracket bears an H or an R¹⁹ substituent, and R¹¹ represents

b1) 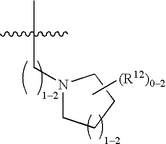
wherein R¹² represents —F, —Cl —Br, —OH, —(C₁₋₃ alkyl) optionally substituted with halogen, —O(C₁₋₃ alkyl) optionally substituted with halogen, or —CH₂OR¹³; wherein R¹³ represents H or —(C₁₋₃ alkyl) optionally substituted with halogen;

b2)

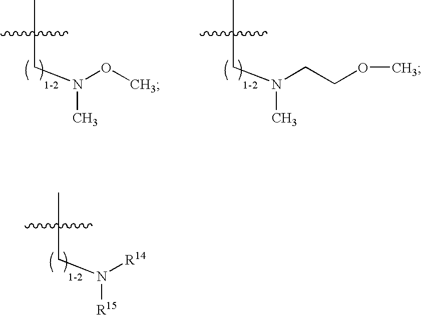

b3)

b4)

wherein R¹⁴ represents H or —(C₁₋₃ alkyl) optionally substituted with halogen; and R¹⁵ represents —(CH₂)₀₋₂(C₃₋₆ cycloalkyl) wherein said cycloalkyl moiety is optionally substituted with up to two substituents independently selected from the group of —F, —Cl, —Br, —OH, —(C₁₋₃ alkyl) optionally substituted with halogen, —O(C₁₋₃ alkyl) optionally substituted with halogen, and —CH₂OR¹⁶, wherein R¹⁶ represents H or —(C₁₋₃ alkyl) optionally substituted with halogen;

b5) 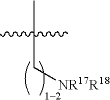
wherein R¹⁷ represents H or —(C₁₋₃ alkyl) optionally substituted with halogen; and R¹⁸ represents —(C₁₋₁₃ alkyl) optionally substituted with halogen; or

b6)

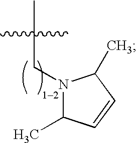

with the proviso that when R¹¹ is —CH₂—NR¹⁷R¹⁸, R¹⁰ is —Br, —CN, —O(C₁₋₃ alkyl), —OCF₃, —OCF₂Cl, or —(C₁₋₃ alkyl) optionally substituted with halogen.

When Z is N, R¹¹ is located on the carbon atom encompassed by the bracket and R¹¹ represents

c1) 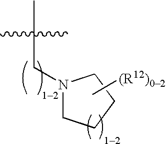
wherein R¹² is as defined above.

R¹⁹ represents —F, —Cl, —Br, —(C₁₋₃ alkyl) optionally substituted with halogen, or —O(C₁₋₃ alkyl) optionally substituted with halogen.

R²⁰ represents —NHR²¹, or —OR²²; wherein R²¹ and R²² are each independently H or —(C₁₋₃ alkyl) optionally substituted with halogen.

Pharmaceutically acceptable salts and hydrates of these materials are also within the scope of the invention.

The invention also relates to a pharmaceutical composition comprising a compound of claim 1 and a pharmaceutically acceptable excipient.

The invention also relates to a method of treating a hyperproliferative disorder comprising administering to a mammalian subject an effective amount of a compound of claim 1.

### DETAILED DESCRIPTION

As stated above, the compounds of the present invention are described by the generalized structural formula (I),

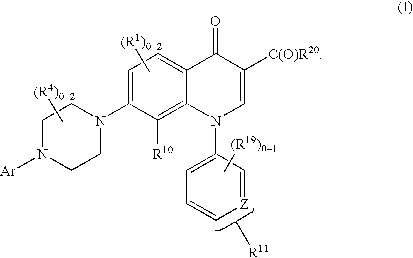

In its broadest embodiment, R¹ represents —F, —Cl, —Br, —NO₂, —(C₁₋₃ alkyl) optionally substituted with halogen, or —NR²R³, in which R² and R³ are independently H or C₁₋₃-alkyl which is optionally substituted with halogen. Preferably, R¹ represents —F, —Cl, —Br, or —(C₁₋₃ alkyl) optionally substituted with halogen. More preferably, R¹ is absent or represents a single substituent selected from —F, —Cl, and —Br.

In its broadest embodiment, R⁴ represents —F, —Cl, —Br, or —(C₁₋₃ alkyl) optionally substituted with halogen. Preferably, R⁴ is absent.

In its broadest embodiment, Ar represents

a1) 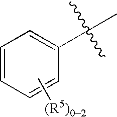
wherein R⁵ represents —F, —Cl, —Br, —(C₁₋₃ alkyl) optionally substituted with halogen, —O(C₁₋₃ alkyl) optionally substituted with halogen, —S(C₁₋₃ alkyl) optionally substituted with halogen, —CN, —C(O)NH₂, —SO₂NH₂, —C(O)CH₃, —NO₂, or —NR⁶R⁷ in which R⁶ and R⁷ are independently H or —(C₁₋₃ alkyl) optionally substituted with halogen;

a2) 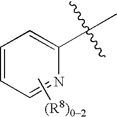
wherein R⁸ represents —CN, —(C₁₋₃ alkyl) optionally substituted with halogen, —O(C₁₋₃ alkyl) optionally substituted with halogen, —C(O)NH₂, or —SO₂NH₂; or

a3) 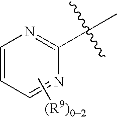
wherein R⁹ represents —F, —Cl, or —Br.

More preferably, Ar represents:

a1) 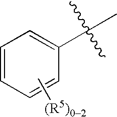
wherein R⁵ represents —F, —Cl, —Br, —(C₁₋₃ alkyl) optionally substituted with halogen, —O(C₁₋₃ alkyl) optionally substituted with halogen, or —CN;

a2) 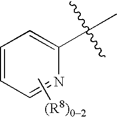
wherein R⁸ represents —CN, —(C₁₋₃ alkyl) optionally substituted with halogen, or —O(C₁₋₃ alkyl) optionally substituted with halogen; or

a3) 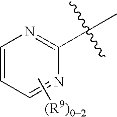
wherein R⁹ represents —F, —Cl, or —Br.

In its broadest embodiment, R¹⁰ represents —Cl, —Br, —(C₁₋₃ alkyl) optionally substituted with halogen, —O(C₁₋₃-alkyl) optionally substituted with halogen, or —CN. Preferably, R¹⁰ represents —Cl, —O(C₁₋₃ alkyl) optionally substituted with halogen, or —CN. More preferably, R¹⁰ represents —Cl, or —O(C₁₋₃ alkyl) optionally substituted with halogen.

In its broadest embodiment, Z represents C or N. Preferably, Z represents C.

In its broadest embodiment, when Z is C, R¹¹ is located on one of the two ring atoms encompassed by the bracket and the other of the two ring atoms encompassed by the bracket bears an H or an R¹⁹ substituent, and R¹¹ represents

b1) 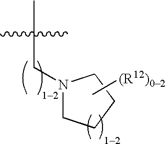
wherein R¹² represents —F, —Cl —Br, —OH, —(C₁₋₃ alkyl) optionally substituted with halogen, —O(C₁₋₃ alkyl) optionally substituted with halogen, or —CH₂OR¹³; wherein R¹³ represents H or —(C₁₋₃ alkyl) optionally substituted with halogen;

b2)

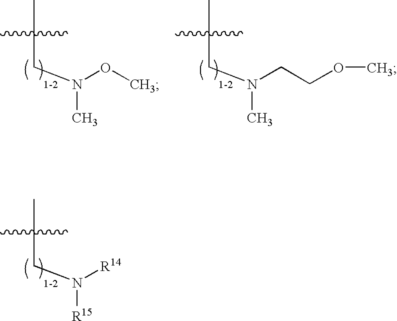

b3)

b4)

wherein R¹⁴ represents H or —(C₁₋₃ alkyl) optionally substituted with halogen; and R¹⁵ represents —(CH₂)₀₋₂(C₃₋₆ cycloalkyl) wherein said cycloalkyl moiety is optionally substituted with up to two substituents independently selected from the group of —F, —Cl, —Br, —OH, —(C₁₋₃ alkyl) optionally substituted with halogen, —O(C₁₋₃ alkyl) optionally substituted with halogen, and —CH₂OR¹⁶, wherein R¹⁶ represents H or —(C₁₋₃ alkyl) optionally substituted with halogen;

b5) 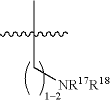
wherein R¹⁷ represents H or —(C₁₋₃ alkyl) optionally substituted with halogen; and R¹⁸ represents —(C₁₋₃ alkyl) optionally substituted with halogen; or

b6)

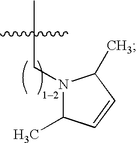

with the proviso that when R¹¹ is —CH₂—NR¹⁷R¹⁸, R¹⁰ is —Br, —CN, —O(C₁₋₃ alkyl), —OCF₃, —OCF₂Cl, or —(C₁₋₃ alkyl) optionally substituted with halogen.

Preferably, R¹¹ is located on one of the two ring atoms encompassed by the bracket and the other of the two ring atoms encompassed by the bracket bears a H or an R¹⁹ substituent, and R¹¹ represents

b1) 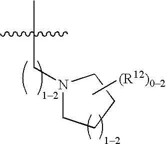
wherein R¹² represents —F, —Cl —Br, —OH, —(C₁₋₃ alkyl) optionally substituted with halogen, —O(C₁₋₃ alkyl) optionally substituted with halogen, or —CH₂OR¹³; wherein R¹³ represents H or —(C₁₋₃ alkyl) optionally substituted with halogen;

b4) 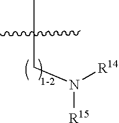
wherein R¹⁴ represents H or —(C₁₋₃ alkyl) optionally substituted with halogen, and R¹⁵ represents —(CH₂)₀₋₂(C₃₋₆ cycloalkyl) wherein the cycloalkyl moiety is optionally substituted with up to two substituents independently selected from the group of —F, —Cl, —Br, —OH, —(C₁₋₃ alkyl) optionally substituted with halogen, —O(C₁₋₃ alkyl) optionally substituted with halogen, and —CH₂OR¹⁶; wherein R¹⁶ represents H or —(C₁₋₃ alkyl) optionally substituted with halogen; or

b5) 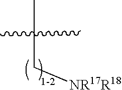
wherein R¹⁷ represents H or —(C₁₋₃ alkyl) optionally substituted with halogen, and R¹⁸ represents —(C₁₋₃ alkyl) optionally substituted with halogen;

with the proviso that when R¹¹ is —CH₂—NR¹⁷R¹⁸, R¹⁰ is —Br, —CN, —O(C₁₋₃ alkyl), —OCF₃, —OCF₂Cl, or —(C₁₋₃ alkyl) optionally substituted with halogen.

More preferably, R¹¹ is located on one of the two ring atoms encompassed by the bracket and the other of the two ring atoms encompassed by the bracket bears a H or an R¹⁹ substituent, and R¹¹ represents

b1)

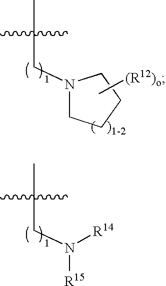

b4)

wherein R¹⁴ represents H or —(C₁₋₃ alkyl) optionally substituted with halogen; and R¹⁵ represents —(CH₂)₀₋₂(C₃₋₆ cycloalkyl) wherein the cycloalkyl moiety is optionally substituted with up to two substituents independently selected from the group of —F, —Cl, —Br, —OH, —(C₁₋₃ alkyl) optionally substituted with halogen, —O(C₁₋₃ alkyl) optionally substituted with halogen, and —CH₂OR¹⁶; wherein R¹⁶ represents H or —(C₁₋₃ alkyl) optionally substituted with halogen; or

b5) 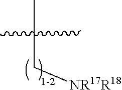
wherein R¹⁷ represents H or —(C₁₋₃ alkyl) optionally substituted with halogen, and R¹⁸ represents —(C₁₋₃ alkyl) optionally substituted with halogen;

with the proviso that when R¹¹ is —CH₂—NR¹⁷R¹⁸, R¹⁰ is —Br, —CN, —O(C₁₋₃ alkyl), —OCF₃, —OCF₂Cl, or —(C₁₋₃ alkyl) optionally substituted with halogen.

In its broadest embodiment, when Z is N, R¹¹ is located on the carbon atom encompassed by the bracket and R¹¹ represents

c1) 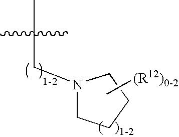
wherein R¹² is as defined above.

In its broadest embodiment, R¹⁹ represents —F, —Cl, —Br, —(C₁₋₃ alkyl) optionally substituted with halogen, or —O(C₁₋₃ alkyl) optionally substituted with halogen. More preferably, R¹⁹ represents —F, —Cl, or —Br.

In its broadest embodiment, R²⁰ represents —NHR²¹ or —OR²²; wherein R²¹ and R²² are each independently H or —(C₁₋₃ alkyl) optionally substituted with halogen. More preferably, R²⁰ represents —OR²²; wherein R²² is H.

In a second and somewhat narrower embodiment, the invention relates to compounds of structural formula (I)

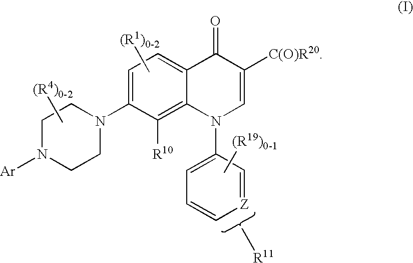

In this second embodiment, the various groups of the formula are defined as follows:

R¹ represents —F, —Cl, —Br, or —(C₁₋₃ alkyl) optionally substituted with halogen.

R⁴ is absent.

Ar represents:

a1) 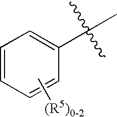
wherein R⁵ represents —F, —Cl, —Br, —(C₁₋₃ alkyl) optionally substituted with halogen, —O(C₁₋₃ alkyl) optionally substituted with halogen, —S(C₁₋₃ alkyl) optionally substituted with halogen, —CN, —C(O)NH₂, —SO₂NH₂, —C(O)CH₃, —NO₂, or —NR⁶R⁷, wherein R⁶ and R⁷ are independently H or —(C₁₋₃ alkyl) optionally substituted with halogen;

a2) 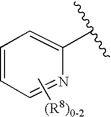
wherein R⁸ represents —CN, —(C₁₋₃ alkyl) optionally substituted with halogen, —O(C₁₋₃ alkyl) optionally substituted with halogen, —C(O)NH₂, or —SO₂NH₂; or

a3) 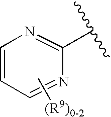
wherein R⁹ represents —F, —Cl, or —Br.

R¹⁰ represents —Cl, —O(C₁₋₃ alkyl) optionally substituted with halogen, or —CN.

Z represents C.

R¹¹ is located on one of the two ring atoms encompassed by the bracket and the other of the two ring atoms encompassed by the bracket bears a H or an R¹⁹ substituent, and R¹¹ represents

b1) 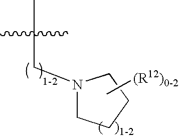
wherein R¹² represents —F, —Cl—Br, —OH, —(C₁₋₃ alkyl) optionally substituted with halogen, —O(C₁₋₃ alkyl) optionally substituted with halogen, or —CH₂OR¹³; wherein R¹³ represents H or —(C₁₋₃ alkyl) optionally substituted with halogen;

b4) 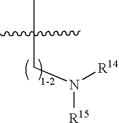
wherein R¹⁴ represents H or —(C₁₋₃ alkyl) optionally substituted with halogen, and R¹⁵ represents —(CH₂)₀₋₂(C₃₋₆ cycloalkyl) wherein the cycloalkyl moiety is optionally substituted with up to two substituents independently selected from the group of —F, —Cl, —Br, —OH, —(C₁₋₃ alkyl) optionally substituted with halogen, —O(C₁₋₃ alkyl) optionally substituted with halogen, and —CH₂OR¹⁶; wherein R¹⁶ represents H or —(C₁₋₃ alkyl) optionally substituted with halogen; or

b5) 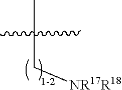
wherein R¹⁷ represents H or —(C₁₋₃ alkyl) optionally substituted with halogen, and R¹⁸ represents —(C₁₋₃ alkyl) optionally substituted with halogen;

with the proviso that when R¹¹ is —CH₂—NR¹⁷R¹⁸, R¹⁰ is —Br, —CN, —O(C₁₋₃ alkyl), —OCF₃, —OCF₂Cl, or —(C₁₋₃ alkyl) optionally substituted with halogen.

R¹⁹ represents —F, —Cl, —Br, —(C₁₋₃ alkyl) optionally substituted with halogen, or —O(C₁₋₃ alkyl) optionally substituted with halogen.

R²⁰ represents —OR²²; wherein R²² is H or —(C₁₋₃ alkyl) optionally substituted with halogen.

In yet another and narrower embodiment, the invention relates to compounds of structural formula (I)

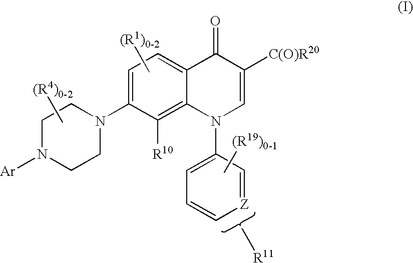

In this formula, the various groups of the formula are defined as follows:

R¹ is absent or represents a single substituent selected from —F, —Cl, and —Br.

R⁴ is absent.

Ar represents:

a1) 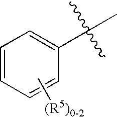
wherein R⁵ represents —F, —Cl, —Br, —(C₁₋₃ alkyl) optionally substituted with halogen, —O(C₁₋₃ alkyl) optionally substituted with halogen, or —CN;

a2) 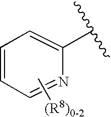
wherein R⁸ represents —CN, —(C₁₋₃ alkyl) optionally substituted with halogen, or —O(C₁₋₃ alkyl) optionally substituted with halogen; or

a3) 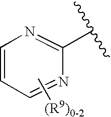
wherein R⁹ represents —F, —Cl, or —Br.

R¹⁰ represents —Cl, or —O(C₁₋₃ alkyl) optionally substituted with halogen.

Z represents C.

R¹¹ is located on one of the two ring atoms encompassed by the bracket and the other of the two ring atoms encompassed by the bracket bears a H or an R¹⁹ substituent, and R¹¹ represents

b1)

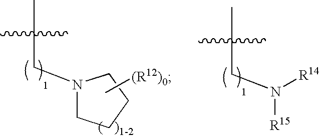

b4)

wherein R¹⁴ represents H or —(C₁₋₃ alkyl) optionally substituted with halogen; and R¹⁵ represents —(CH₂)₀₋₂(C₃₋₆ cycloalkyl) wherein the cycloalkyl moiety is optionally substituted with up to two substituents independently selected from the group of —F, —Cl, —Br, —OH, —(C₁₋₃ alkyl) optionally substituted with halogen, —O(C₁₋₃ alkyl) optionally substituted with halogen, and —CH₂OR¹⁶; wherein R¹⁶ represents H or —(C₁₋₃ alkyl) optionally substituted with halogen; or

b5) 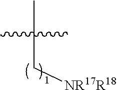
wherein R¹⁷ represents H or —(C₁₋₃ alkyl) optionally substituted with halogen, and R¹⁸ represents —(C₁₋₃ alkyl) optionally substituted with halogen;

with the proviso that when R¹¹ is —CH₂—NR¹⁷R¹⁸, R¹⁰ is —Br, —CN, —O(C₁₋₃ alkyl), —OCF₃, —OCF₂Cl, or —(C₁₋₃ alkyl) optionally substituted with halogen.

R¹⁹ represents —F, —Cl, or —Br.

R²⁰ represents —OR²²; wherein R²² is H.

Definitions of Terms

The term “C₁₋₃ alkyl” means linear or branched saturated hydrocarbon groups having from 1 to 3 carbon atoms. Such groups include but methyl, ethyl, n-propyl, and isopropyl.

The term “C₁₋₃ alkoxy” means a linear or branched saturated hydrocarbon group having from 1 to 8 carbon atoms, attached to an O atom. The O atom is the point of attachment of the alkoxy substituent to the rest of the molecule. Such groups include methoxy, ethoxy, n-propoxy, and isopropoxy.

The term “C₃₋₆ cycloalkyl” means a cyclic hydrocarbon ring containing from 3 to 6 carbons. Such groups include cyclopropyl, cyclobutyl, cyclopentyl, and cyclohexyl.

The term “halogen” in the present application connotes an atom selected from F, Cl, and Br, where Cl, and F are preferred and F is most preferred.

The language “optionally substituted with halogen” means that the moiety under consideration may be unsubstituted or may bear from 1 up to the maximum possible number of halogen substituents. The halogen substituents may be the same or different.

Compounds of Formula I are prepared by the methods shown in Reaction Scheme 1, shown below.

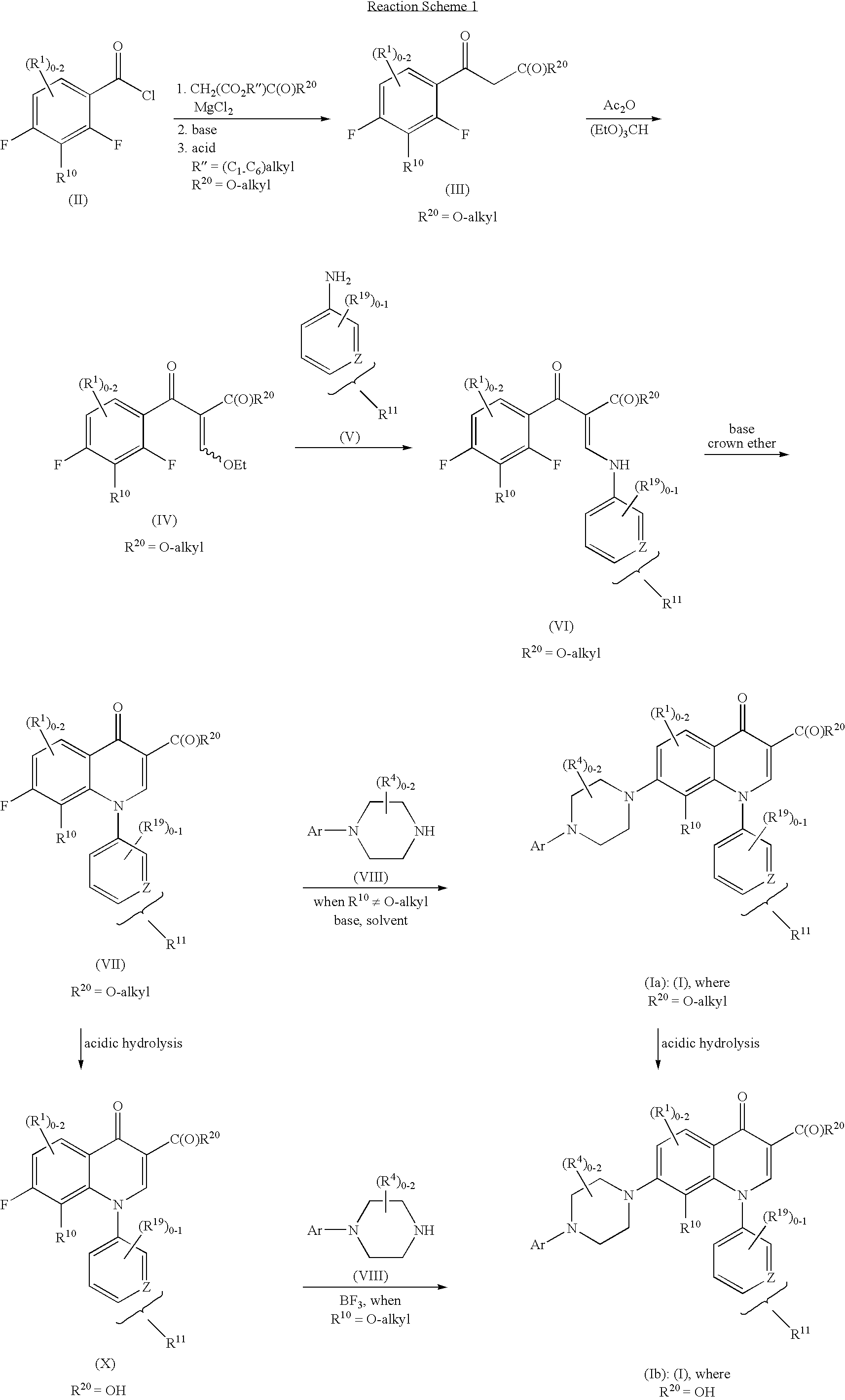

As shown in Reaction Scheme 1, preparation of Formula (I) compounds is accomplished from key starting materials of Formula (IV), Formula (V) and Formula (VII).

The ethoxymethylene ketoesters of Formula (IV) are either commercially available or prepared from the corresponding acid chloride (II). Reaction of (II) with a malonic ester, facilitated by MgCl₂, gives, after aqueous base hydrolysis and acidic (e.g., TsOH) decarboxylation, the intermediate ketoester (III). Condensation of (IM with triethylorthoformate in the presence of a dehydrating agent such as acetic anhydride provides the compounds of Formula (IV).

Reaction of (IV) with the amino compounds of Formula (V) is accomplished in either an inert solvent or neat, and provides the enaminoketoester of Formula (VI). Cyclization of (VI) to the quinolone of Formula (VII) is accomplished by the reaction in a base such as potassium carbonate, and is facilitated by addition of an appropriate crown ether (e.g., 18-crown-6).

For the preparation of Formula (I) compounds where R¹⁰ is other than an alkoxy group, the piperazine of Formula (VIII) is allowed to react with quinolone (VII) in a polar solvent such as DMSO, facilitated by a non-nucleophilic base such as diisopropylethylamine (Hunig's base, DIEA), to produce the ester of Formula (Ia) [(I) where R is O-alkyl]. Acid hydrolysis of (Ia) (e.g. aqueous HCl) provides the compounds of Formula (Ib), [(I) where R²⁰ is OH and where R² is other than an alkoxy or haloalkoxy group].

For the preparation of Formula (I) compounds where R¹⁰ is an alkoxy group, the quinoline of Formula (VII) is first hydrolyzed (e.g, aq HCl) to the acid of Formula (X), which in turn is allowed to react with the piperazine of Formula (VIII) in the presence of a complexing agent, e.g., BF₃, to provide the Formula (Ib) compounds [(I) where R²⁰ is OH and where R¹⁰ is an alkoxy group.

Formula (Ia) and Formula (Ib) compounds are interconvertable, as desired, by conducting hydrolysis, or by esterification reactions with the appropriate alcohol of formula R²⁰OH. Formula (I) compounds in which R²⁰ is NHR²¹ are prepared by reaction of the appropriate amine of Formula R²¹NH₂ with the carboxylic acid compound of Formula (Ib) either directly or by conversion of (Ib) to an acid chloride or mixed anhydride. Alternatively, the compounds of Formula (I) in which R²⁰ is NHR²¹, can be prepared by heating the Formula (Ia) ester compounds with the amines of Formula R²¹NH₂. by standard procedures known in the art.

The anilines or amino pyridines of Formula (V) are either commercially available are prepared by standard methods known to those skilled in the art, for example, reduction of the corresponding nitro compounds.

The aryl piperazines of Formula (VIII) are either commercially available, or prepared from standard methods known to those skilled in the art such as coupling of an aryl halide with piperazine.

By utilizing the methods described the above general scheme and selecting the appropriate starting materials, compounds of the invention can be made. Details of the conditions used for the preparation of representative examples of invention are described below in the experimental procedures.

It is to be understood that sensitive or reactive substituents attached to intermediates or to compounds of Formula (I) may need to be protected and deprotected during the preparations described above. Protecting groups in general may be added and removed by conventional methods well known in the art [see, e.g., T. W. Greene and P. G. M. Wuts, Protective Groups in Organic Synthesis; Wiley: New York, (1999)].

The use of a pharmaceutically acceptable salt of the compounds of this invention is also within the scope of this invention. The term “pharmaceutically acceptable salt” refers to either inorganic or organic salts of a compound of the present invention that have properties acceptable for the therapeutic use intended. For example, see: S. M. Berge, et al. “Pharmaceutical Salts,” J. Pharm. Sci. 1977, 66, 1-19.

Representative salts of the compounds of this invention also include the conventional non-toxic salts and the quaternary ammonium salts that are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate. The term acid addition salts also comprises the hydrates and the solvent addition forms which the compounds of this invention are able to form. Examples of such forms are, for example, hydrates, alcoholates and the like.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates including dimethyl, diethyl, and dibutyl sulfate; and diamyl sulfates, long chain halides such as decyl, lauryl, myristyl and stearyl chlorides, bromides and iodides, aralkyl halides including benzyl and phenethyl bromides, and others.

The esters of a compound of this invention are non-toric, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

The compounds used in this invention may contain one or more asymmetric centers, depending upon the location and nature of the various substituents desired. Asymmetric carbon atoms may be present in the (R)- or (S)-configuration or may be mixtures of compounds with the (R)- and (S)-configurations. In certain instances, asymmetry may also be present due to restricted rotation about a given bond, for example, the central bond adjoining two substituted aromatic rings of the specified compounds. It is intended that all such configurations (including enantiomers and diastereomers) are included within the scope of the present invention. Preferred compounds are those with the absolute configuration of the compound of this invention which produces the more desirable biological activity. Separated, pure or partially purified isomers or racemic mixtures of the compounds of this invention are also included within the scope of the present invention.

The following specific examples are presented to illustrate the invention described herein, but should not be construed as limiting the scope of the invention in any way.

### EXPERIMENTAL EXAMPLES OF THE INVENTION

General. All amines and arylpiperazines used in these experiments were purchased from commercial sources as listed in Table 1.

TABLE 1
Reference Table for Sources and Preparative Methods of Starting Materials

| Structure | Source |
|---|---|
| 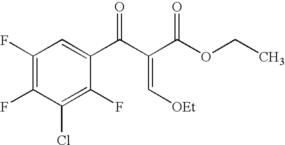 | Obtained from Bayer Chemicals, Germany. For preparation, see J. Med. Chem. 1994, 37, 3344. |
| 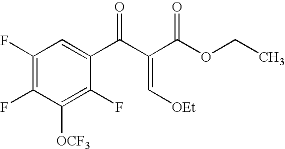 | Obtained from Bayer Chemicals, Germany. See Intermediate E, Step 2, for an experimental preparation |
| 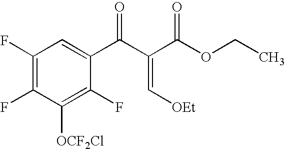 | Obtained from Bayer Chemicals, Germany. See Intermediate D, step 4 for an experimental preparation |
| 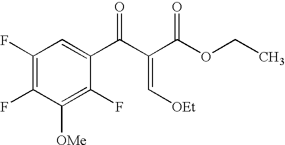 | Obtained from Bayer Chemicals, Germany. |
| 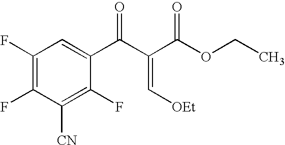 | Obtained from Bayer Chemicals, Germany. |
| 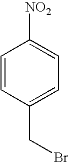 | Commercially available from Sigma-Aldrich Company, Milwaukee, WI, USA |
| 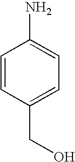 | Commercially available from Sigma-Aldrich Company, Milwaukee, WI, USA |
| 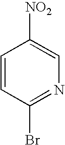 | Commercially available from Sigma-Aldrich Company, Milwaukee, WI, USA |
| 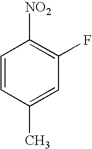 | Commercially available from Sigma-Aldrich Company, Milwaukee, WI, USA |
| 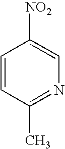 | 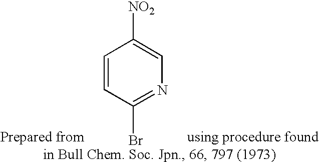 |

HPLC-electrospray mass spectra (HPLC ES-MS) were obtained using a Hewlett-Packard 1100 HPLC equipped with a quaternary pump, a variable wavelength detector, a YMC Pro C18 2.0 mm×23 mm column, and a Finnigan LCQ ion trap mass spectrometer with electrospray ionization. Gradient elution from 90% A to 95% B over 4 minutes was used on the HPLC. Buffer A was 98% water, 2% Acetonitrile, and 0.02% TFA, and Buffer B was 98% Acetonitrile, 2% water, and 0.018% TFA. Spectra were scanned from 140-1200 amu using a variable ion time according to the number of ions in the source.

Proton (¹H) nuclear magnetic resonance (NMR) spectra were measured with a General Electric GN-Omega 300 (300 MHz) spectrometer with either Me₄Si (δ0.00) or residual protonated solvent (CHCl₃ δ 7.26; MeOH δ 3.30; DMSO δ 2.49) as standard. Carbon (¹³C) NMR spectra were measured with a General Electric GN-Omega 300 (75 MHz) spectrometer with solvent (CDCl₃ δ 77.0; d₃-MeOD; δ 49.0; d₆-DMSO δ 39.5) as standard.

Chiral separations were performed using a commercially available Chiracel® AD HPLC column, eluting with a gradient of isopropanol in hexane (from 1% to 15%) with addition of 0.1% trifluoroacetic acid.

### Abbreviations and Acronyms

When the following abbreviations are used herein, they have the following meanings:

Abs absolute

Ac₂O acetic anhydride

anhy anhydrous

aq aqueous

calcd calculated

Celite® diatomaceous earth filter agent, ®Celite Corp.

conc concentrated

d day(s)

DABCO 1,4-diazabicyclo[2.2.2]octane

DBU 1,8-diazabicyclo[5.4.0]undec-7-ene

DIA diisopropylamine

DIEA diisopropylethylamine (Hunig's Base)

DMF N,N-dimethylformamide

DMSO dimethylsulfoxide

EtOAc ethyl acetate

EtOH ethanol (100%)

Et₂O diethyl ether

h hour(s)

¹H NMR proton nuclear magnetic resonance spectroscopy

HPLC high performance liquid chromatography

IPA isopropylamine

LC-MS liquid chromatography-mass spectrometry

m/z mass-to-charge ratio

MeOH methanol

min minute(s)

MTBE tert-butyl methyl ether

NMR nuclear magnetic resonance spectroscopy

R_{f} retention factor (TLC)

RT retention time (HPLC)

rt room temperature

SM starting material

TEA triethylamine

THF tetrahydrofuran

TFA trifluoroacetic acid

TLC thin layer chromatography

Preparation of Intermediates

### Intermediate A: Preparation of 4-pyrrolidin-1-ylmethyl-phenylamine

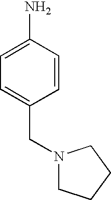

### Step 1: Preparation of 1-(4-nitrobenzyl)-pyrrolidine

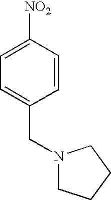

To a solution of 4-nitrobenzyl bromide (5 g) in THF (40 mL) pyrrolidine (5.5 mL) was added at once at rt and the mixture was stirred for 3 h. TLC (EtOAc) showed a single slow moving spot appeared and no SM was observed. The reaction mixture was concentrated to remove solvent and the residue was partitioned between EtOAc and water. The organic phase was dried with MgSO₄, filtered and concentrated to provide crude 1-(4-nitrobenzyl)-pyrrolidine.

### Step 2: Preparation of the title compound 4-pyrrolidin-1-ylmethyl-phenylamine

The crude product from step 1 was dissolved in EtOAc and was hydrogenated using Raney-Ni as catalyst and under atmospheric pressure of hydrogen. The reaction mixture was stirred overnight at rt. TLC showed a new slow moving spot. The precipitate was filtered off, and solution was concentrated to give 3.5 g of 4-pyrrolidin-1-ylmethyl-phenylamine as oil. (86% yield) ¹H NMR (DMSO, ppm): δ 6.88 (d, 2H), 6.45 (d, 2H), 4.88 (s, 2H), 2.32 (m, 4H), 1.64 (m, 4H). LC-MS: 177 [M+H]⁺

### Intermediate B: Preparation of ethyl 8-chloro-6,7-difluoro-4-oxo-1-[4-(pyrrolidin-1-ylmethyl)phenyl]-1,4-dihydroquinoline-3-carboxylate

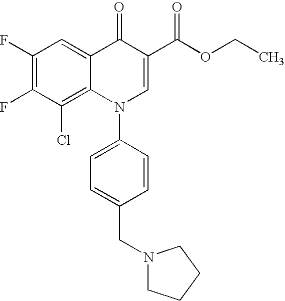

To the solution of ethyl 2-(3-chloro-2,4,5-trifluorobenzoyl)-3-ethoxyacrylate (0.5 g) in EtOH (15 mL) at below −10° C. was added 4-pyrrolidin-1-ylmethyl-phenylamine (Intermediate A, 0.26 g). The reaction was warmed to rt and stirred for an additional 3 h. TLC (MeOH—CH₂Cl₂, 1:9) showed a new spot formed. The solvent was removed and the residue was dissolved with THF (20 mL) and treated with 18-crown-6 (0.19 g) and potassium carbonate (0.41 g) and refluxed for 4 h. The solution was cooled to rt and the resulting precipitate was filtered off and solvent was concentrated. The residue was purified on a silica gel column using Combiflash (10 g silica gel) eluted with MeOH in CH₂Cl₂ from 0% to 10%. After the solvent was removed, 0.3 g of yellow precipitate was obtained (50% yield). LC-MS showed a desired product with m/z [M+H]⁺ 447. ¹H NMR (CDCl₃): δ 1.40 (t, 3H); 1.84 (m, 4H); 2.56 (m, 4H); 3.72 (s, 2H); 4.38 (q, 2H); 7.28-7.49 (q, 4H); 8.33 (dd, 1H); 8.46 (s, 1H).

### Intermediate C: Preparation of ethyl 6,7-difluoro-8-methoxy-4-oxo-1-[4-(pyrrolidin-1-ylmethyl)phenyl]-1,4-dihydroquinoline-3-carboxylate

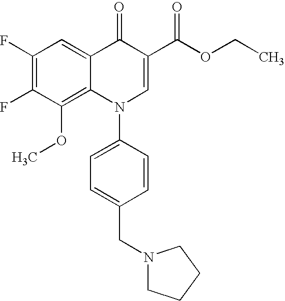

Diisopropylethylamine (3.7 mL, 18 mmol) was added to a solution of 4-pyrrolidin-1-ylmethyl-phenylamine (intermediate A, 1.59 g, 9.0 mmol) in DMSO (100 mL). Ethyl (2Z)-3-ethoxy-2-(2,4,5-trifluoro-3-methoxybenzoyl)acrylate (3.0 g, 9.0 mmol) was then added followed by DBU (2.65 mL, 18 mmol) to the mixture at rt. The reaction mixture turned dark brown. After stirring for 2.5 h at rt a precipitate formed. Water (100 mL) was added to the precipitate. After 10 min of stirring, the solids were filtered off and rinsed with copious amounts of water, followed by a small ether rinse to give 3.06 g (77%) of title compound ethyl 6,7-difluoro-8-methoxy-4-oxo-1-[4-(pyrrolidin-1-ylmethyl)phenyl]-1,4-dihydro-quinoline-3-carboxylate as a colorless solid. ¹H NMR (CDCl₃): δ 8.2 (s, 1H), 7.9 (t, 1H), 7.5-7.4 (dd, 4H), 4.2 (q, 2H), 3.7 (s, 2H), 3.3 (s, 3H), 2.4 (m, 4H), 1.7 (s, 4H), 1.3-1.2 (t, 3H).

### Intermediate D: Preparation of ethyl 8-[chloro(difluoro)methoxy]-6,7-difluoro-4-oxo-1-[4-(pyrrolidin-1-ylmethyl)phenyl]-1,4-dihydroquinoline-3-carboxylate

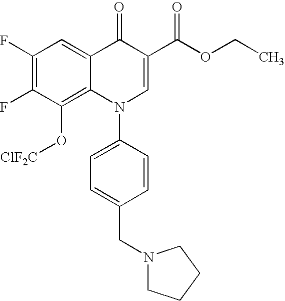

### Step 1: Synthesis of 2,4,5-Trifluoro-3-(trichloromethoxy)benzoyl chloride

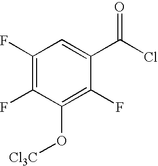

2,4,5-Trifluoro-3-methoxybenzoylchloride (400 g, 1.78 mol) was dissolved in 4-chlorobenzotrifluoride (1000 mL). 2 g of PCl₃ was then added and the mixture was heated to 125° C. At this temperature chlorine was passed into the solution under irradiation until complete conversion of the starting material (checked by GC). After eight days, solvent was removed by distillation under reduced pressure to provide 2,4,5-trifluoro-3-(trichloromethoxy)benzoyl chloride (crude yield 741 g).

### Step 2: Synthesis of 2,4,5-Trifluoro-3-(chlorodifluoromethoxy)benzoyl fluoride

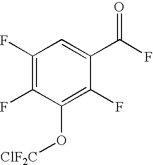

Hydrogen fluoride (188 mL) and antimony(V)chloride (3 g) were mixed in a steel vessel. To the vessel 2,4,5-trifluoro-3-(trichloromethoxy)benzoyl chloride (216 g, 0.66 mol) was added in small portions. The vessel was closed, pressurized with 10 bar of nitrogen and heated to 130° C. for 14 hours. At rt the contents of the vessel were poured onto ice and extracted several times with CH₂Cl₂. The organic layers were combined, dried and the solvent was removed under reduced pressure. The product 2,4,5-trifluoro-3-(chlorodifluoro-methoxy)benzoyl fluoride was purified by distillation, bp 80° C. at 20 mbar (yield: 145 g)

### Step 3: Synthesis of Ethyl 3-oxo-3-[2,4,5-trifluoro-3-(chlorodifluoro-methoxy)phenyl]-propanoate

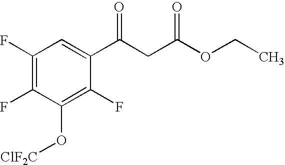

MgCl₂ (39.2 g, 0.410 mol) was dissolved in acetonitrile (200 mL) at 0° C. After 1 h, diethyl malonate (69 g, 0.431 mol) was added at rt. The mixture was cooled to 0° C. and over a period of 2 h, triethylamine (113 mL, 0.813 mol) was added. Then 2,4,5-trifluoro-3-(chlorodifluoromethoxy)-benzoyl chloride (120 g, 0.407 mol) was added to the solution at 0° C. and stirred further for 4 h at 0° C. The reaction mixture was allowed to warm up to room temperature and stirred for a further 24 h. The reaction was worked up by addition of 400 mL 5N HCl, extracted several times with MTBE, dried, and the solvent was removed. This crude material was then suspended in 380 mL water, 4-toluenesulfonic acid (4 g) was added and the suspension was heated to reflux for 8 hours. After cooling to room temperature the organic layer was separated, the aqueous was extracted with CH₂Cl₂. The combined organic layer was dried and the solvent was removed. Crude yield of ethyl-3-oxo-3-[2,4,5-trifluoro-3-(chlorodifluoro-methoxy)phenyl]-propanoate 123 g.

### Step 4: Synthesis of Ethyl-3-ethoxy-2-[2,4,5-trifluoro-3-(chloro-difluoromethoxy)benzoyl]-acrylate

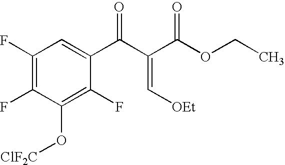

Ethyl-3-oxo-3-[2,4,5-trifluoro-3-(chlorodifluoromethoxy)phenyl]-propanoate (step 3 product, 110 g, 0.317 mol) was mixed with 77 mL (0.825 mol) acetic acid anhydride. Triethyl orthoformate (75 g, 0.507 mol) was then added at rt and the reaction mixture was heated to 120° C. for 10 h. The reagent and solvent were evaporated and the product (ethyl-3-ethoxy-2-[2,4,5-trifluoro-3-(chlorodifluoromethoxy)-benzoyl]acrylate, yield 111 g) was dried in vacuum.

### Step 5: Preparation of ethyl (2Z)-2-{3-[chloro(difluoro)methoxy]-2,4,5-trifluorobenzoyl}-3-{[4-(pyrrolidin-1-ylmethyl)phenyl]amino}acrylate

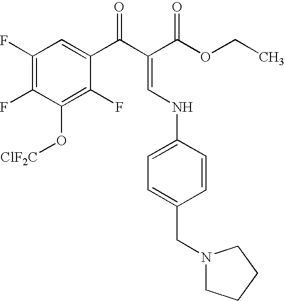

Ethyl-3-ethoxy-2-[2,4,5-trifluoro-3-(chlorodifluoromethoxy)benzoyl]-acrylate (step 4 product, 3 g, 7.77 mmol) was dissolved in ethanol (50 mL). 4-pyrrolidin-1-ylmethyl-phenylamine (Intermediate A, 1.24 g, 7.06 mmol) was added to the solution at −10° C. The reaction mixture was stirred at rt for 2.5 h, the solution was evaporated to dryness and the desired product was collected after a short silica gel column using from 50% ethyl acetate in hexane to 100% ethyl acetate in hexane. This product was 90% pure by LC-MS and submitted for the next step without further purification.

### Step 6: Preparation of the title compound, ethyl 8-[chloro(difluoro)methoxy]-6,7-difluoro-4-oxo-1-[4-(pyrrolidin-1-ylmethyl)phenyl]-1,4-dihydroquinoline-3-carboxylate

A solution of ethyl (2Z)-2-{3-[chloro(difluoro)methoxy]-2,4,5-trifluorobenzoyl}-3-{[4-(pyrrolidin-1-ylmethyl)phenyl]amino}acrylate (step 5 product, 4 g, 7.5 mmol), Potassium carbonate (2.93 g, 21.2 mmol), 18-crown-6 (0.56 g, 2.12 mmol), in acetonitrile (50 mL) was refluxed for 4 h, then cooled to room temperature, filtered, concentrated and the pure product (2.2 g, 57% yield) was purified by short silica gel column eluted with methanol/dichloromethane (3/97). ¹H NMR (CD2Cl2): δ 8.42(s, 1H), 8.37(t, 1H), 7.64(br, 2H), 7.32(d, 2H), 4.39(q, 2H), 3.88(br, 2H), 2.75(br, 4H), 1.94(br, 4H), 1.40(t, 3H).

### Intermediate E: Preparation of ethyl 8-[trifluoromethoxy]-6,7-difluoro-4-oxo-1-[4-(pyrrolidin-1-ylmethyl)phenyl]-1,4-dihydroquinoline-3-carboxylate

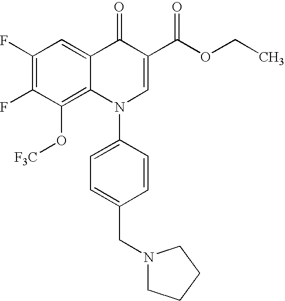

### Step 1: Synthesis of 2,4,5-Trifluoro-3-(trifluoromethoxy)benzoyl fluoride

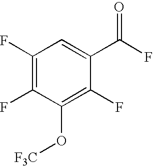

Hydrogen fluoride (264 mL) and antimony(V)chloride (6 g) were mixed in a steel vessel. 2,4,5-Trifluoro-3-(chlorodifluoromethoxy)benzoyl fluoride (Intermediate B, step 2 product, 141 g, 0.5 mol) was then added in small portions. The vessel was closed, pressurized with 10 bar nitrogen and heated to 160° C. for 14 h. At room temperature the contents of the vessel were poured on ice and extracted several times with CH₂Cl₂. The organic layers were combined, dried and the solvent was removed under reduced pressure. The desired product 2,4,5-trifluoro-3-(trifluoromethoxy)benzoyl fluoride was obtained by distillation (80° C., 60 mbar, yield 57 g).

### Step 2: Synthesis of ethyl-3-ethoxy-2-[2,4,5-trifluoro-3-(trifluoromethoxy)benzoyl]-acrylate

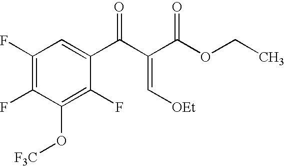

Synthesis of title compound was achieved by following the protocol as described in the synthesis of Intermediate D using 2,4,5-trifluoro-3-(trifluoromethoxy)benzoyl fluoride instead of 2,4,5-trifluoro-3-(chloro-difluoromethoxy)benzoyl fluoride in step 3 of Intermediate D synthesis.

### Step 3: Synthesis of ethyl-3-{[4-(pyrrolidin-1-ylmethyl)phenyl]amino}-2-[2,4,5-trifluoro-3-(trifluoromethoxy)benzoyl]acrylate

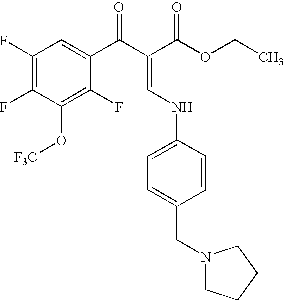

Ethyl-3-ethoxy-2-[2,4,5-trifluoro-3-(trifluoromethoxy)benzoyl]acrylate (2.0 g, 5.2 mmol, step 2 product) was dissolved in EtOH (abs., 50.0 mL) then cooled to 0° C. [4-(Pyrrolidin-1-ylmethyl)phenyl]amine dihydrochloride (1.29 g, 5.2 mmol, Intermediate A) and triethylamine (2.17 mL, 15.5 mmol, 3 equiv.) were added and the mixture was allowed to warm to room temperature and stir for 2 h. LC-MS analysis showed the reaction was complete. Solvent was removed in vacuo and the ethyl-3-{[4-(pyrrolidin-1-ylmethyl)phenyl]amino}-2-[2,4,5-trifluoro-3-(tri-fluoromethoxy)benzoyl]acrylate was used without further purification.

### Step 4: Synthesis of the title compound, ethyl 8-[trifluoromethoxy]-6,7-difluoro-4-oxo-1-[4-(pyrrolidin-1-ylmethyl)phenyl]-1,4-dihydroquinoline-3-carboxylate

Ethyl-3-{[4-(pyrrolidin-1-ylmethyl)phenyl]amino}-2-[2,4,5-trifluoro-3-(tri-fluoromethoxy)benzoyl]acrylate (approx 5.2 mmol) was dissolved in THF (10 mL) and K₂CO₃ (2.15 g, 15.5 mmol, 3 equiv.) and 18-crown-6 (957 mg, 1.55 mmol, 0.3 equiv.) were added. The mixture was heated to reflux for 2 h; LC-MS showed the starting material was gone. The reaction was cooled to room temperature, filtered and the solids were rinsed with THF. The solvent was removed from the filtrate in vacuo and hexanes were added to the oil. The precipitate was filtered and dried in a drying oven to give 800 mg (32%, steps 3 and 4 combined) of ethyl 6,7-difluoro-4-oxo-1-[4-(pyrrolidin-1-ylmethyl)phenyl]-8-(trifluoromethoxy)-1,4-dihydroquinoline-3-carboxylate as an off-white solid. LC-MS RT 2.13 min; [M+H]⁺ 497.8.

### Intermediate F: Preparation of ethyl 8-chloro-6,7-difluoro-1-[4-(hydroxymethyl)-phenyl]-4-oxo-1,4-dihydroquinoline-3-carboxylate

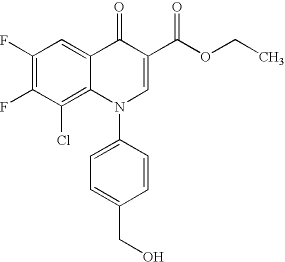

### Step 1: Synthesis of ethyl 2-(3-chloro-2,4,5-trifluorobenzoyl)-3-{[4-(hydroxymethyl)phenyl]amino}acrylate

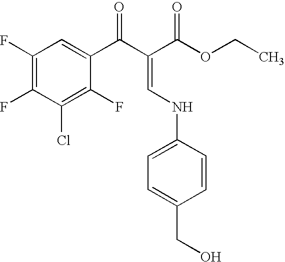

To a solution of ethyl 2-(3-chloro-2,4,5-trifluorobenzoyl)-3-ethoxyacrylate (15 g, 44.55 mmol) in EtOH (500 mL) was added a solution of 4-aminobenzyl alcohol (5.50 g, 44.66 mmol) in EtOH at about −30° C. Light yellow solid formed at the end of the addition. The reaction was then stirred at rt for 2 h. Solvent was then removed under reduced pressure and 100 mL of 2-propanol was added. The flask was placed in the refrigerator overnight. The yellow solid formed was filtered and then washed with Hexane/2-propanol (90/10), and then hexane to give desired product as a yellow powder (14.4 g, 78%). ¹H NMR (DMSO-d₆): δ12.32 (d, 1H); 8.57 (d, 1H); 7.66 (m, 1H); 7.49 (m, 2H); 7.37 (m, 2H); 5.25 (m, 1H); 4.94 (m, 2H); 4.04 (m, 2H); 1.04 (t, 3H). MS [M+H]⁺: 414.0 m/z. Calcd 413. RT (LC-MS): 3.40 min. TLC (CH₂Cl₂/2M NH₃ in MeOH 95/5) R_{f}=0.32

### Step 2: Synthesis of the title compound ethyl 8-chloro-6,7-difluoro-1-[4-(hydroxymethyl)phenyl]-4-oxo-1,4-dihydroquinoline-3-carboxylate

To a solution of ethyl 2-(3-chloro-2,4,5-trifluorobenzoyl)-3-{[4-(hydroxy-methyl)phenyl]amino}acrylate (14 g, 33.84 mmol) in THF (600 mL) was added K₂CO₃ (14.03 g, 101.5 mmol) and 18-crown-6 (2.68 g, 10.15 mmol). The mixture was stirred at rt for about 40 min (check TLC until all the SM consumed). The solid was filtered. The solvent was removed. The crude product was purified by column (CH₂Cl₂ with 1-3% of 2M NH₃ in methanol) and then recrystallized from ethyl acetate/hexane to give ethyl 8-chloro-6,7-difluoro-1-[4-(hydroxy-methyl)phenyl]-4-oxo-1,4-dihydroquinoline-3-carboxylate as a off white powder (12.8 g, 96%). ¹H NMR (DMSO-d₆): δ 8.34 (s, 1H); 8.21 (t, 1H); 7.56 (d, 2H, J=8); 7.48 (d, 2H, J=8); 5.38 (t 1H); 4.60 (d, 2H, J=4.8); 4.23 (q, 2H); 1.26 (t, 3H). MS [M+H]⁺: 394.4 m/z. Calcd 393. RT (LC-MS): 2.54 min. TLC (CH₂Cl₂/2M NH₃ in MeOH 95/5) R_{f}=0.31

### PREPARATIVE EXAMPLES OF THE INVENTION

### Example 1

### Preparation of 8-chloro-6-fluoro-4-oxo-7-(4-pyridin-2-yl-piperazin-1-yl)-1-[4-(pyrrolidin-1-ylmethyl)phenyl]-1,4-dihydroquinoline-3-carboxylic acid

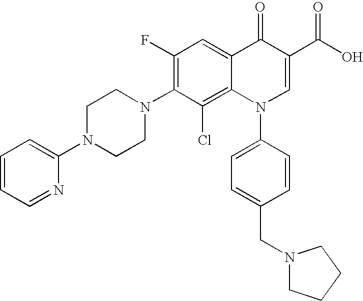

### Step 1: Preparation of ethyl 8-chloro-6-fluoro-4-oxo-7-(4-pyridin-2-ylpiperazin-1-yl)-1-[4-(pyrrolidin-1-ylmethyl)phenyl]-1,4-dihydroquinoline-3-carboxylate

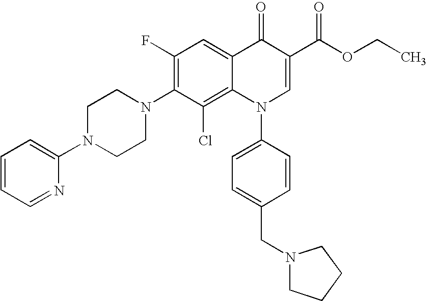

A solution of ethyl 8-chloro-6,7-difluoro-4-oxo-1-[4-(pyrrolidin-1-ylmethyl)phenyl]-1,4-dihydroquinoline-3-carboxylate (Intermediate B, 3.0 g), 1-(2-pyridylphenyl)piperazine (2.2 g), and DIEA (2.4 mL) in DMSO (45 mL) was heated at 100° C. overnight. The reaction mixture was cooled to rt, resulting in formation of precipitate. The precipitate was filtered, washed with isopropanol and dried under high vacuum at 50° C. overnight to give 2.62 g of desired product (66% yield). ¹H NMR (CDCl₃): δ 8.45 (s, 1H), 8.16 (m, 2H), 7.5 (m, 3H), 7.24 (m, 2H), 6.6 (m, 2H), 4.4 (q, 2H), 3.8 (s, 2H), 3.6 (wide, 4H), 3.4 (s, 4H), 2.7 (s, 4H), 1.9 (s, 4H), 1.4 (t, 3H). LC-MS: 590 [M+H]⁺, RT 1.75 min.

### Step 2: Preparation of the title compound, 8-chloro-6-fluoro-4-oxo-7-(4-pyridin-2-ylpiperazin-1-yl)-1-[4-(pyrrolidin-1-ylmethyl)phenyl]-1,4-dihydro-quinoline-3-carboxylic acid

A mixture of step 1 product (2.37 g) in a mixed solvent consisting of isopropanol (77 mL), H₂O (7 mL), and HCl (conc., 15 mL) was heated at 100° C. overnight. LC-MS showed a single pure product peak with [M+H]⁺ at m/z 562. After the solution was cooled to rt, a yellow precipitate was formed. The precipitate was filtered, washed with isopropanol 3 times, and dried under high vacuum at 50° C. overnight to give 1.7 g of yellow product. ¹H NMR (DMSO): δ 11.3 (s, 1H), 8.6 (s, 1H), 8.15 (d, 1H), 8.05 (d, 1H), 7.9 (s, 1H), 7.85 (d, 2H), 7.7 (d, 2H), 7.3 (d, 1H), 6.9 (t, 1H), 4.45 (d, 2H), 3.8 (s, 2H), 3.7 (wide, 4H), 3.3 (s, 4H), 3.1 (m, 2H), 2.04 (m, 2H), 1.9 (m, 2H). LC-MS: 562 [M+H]⁺, RT 1.83 min

### Example 2

### Preparation of 8-chloro-6-fluoro-7-[4-(4-fluorophenyl)-piperazin-1-yl]-4-oxo-1-[4-(pyrrolidin-1-ylmethyl)phenyl]-1,4-dihydroquinoline-3-carboxylic acid

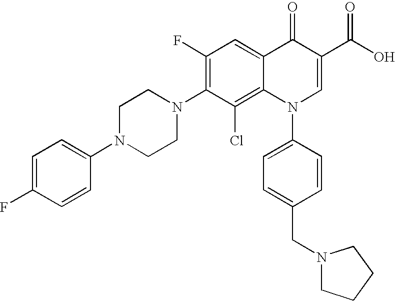

Example 2 was prepared using the procedure as described for Example 1, using 1-(4-fluorophenyl)piperazine in step 1. LC-MS: 579 [M+H]⁺, RT 2.56 min.

### Example 3

### Preparation of 8-chloro-6-fluoro-7-[4-(4-chlorophenyl)-piperazin-1-yl]-4-oxo-1-[4-(pyrrolidin-1-ylmethyl)phenyl]-1,4-dihydroquinoline-3-carboxylic acid

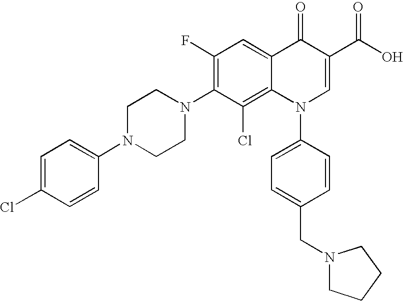

Example 3 was prepared using the procedure as described for Example 1, using 1-(4-chlorophenyl)piperazine in step 1. LC-MS: 595 [M+H]⁺, RT 2.92 min.

### Example 4

### Preparation of 8-chloro-6-fluoro-7-[4-(2-cyanophenyl)-piperazin-1-yl]-4-oxo-1-[4-(pyrrolidin-1-ylmethyl)phenyl]1,4-dihydroquinoline-3-carboxylic acid

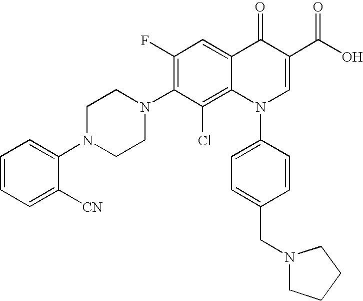

Example 4 was prepared using the procedure as described for Example 1, using 2-piperazin-1-ylbenzonitrile in step 1. LC-MS: 586 [M+H]⁺, RT 2.58 min.

### Example 5

### Preparation of 8-chloro-7-{4-[2-cyano-4-(trifluoromethyl)-phenyl]piperazin-1-yl}-6-fluoro-4-oxo-1-[4 (pyrrolidin-1-ylmethyl)phenyl]-1,4-dihydroquinoline-3-carboxylic acid

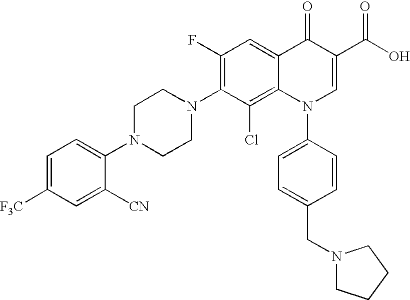

Example 5 was prepared using the procedure as described for Example 1, using 1-(2-cyano-4-(trifluoromethyl)phenyl)piperazine in step 1. LC-MS: 682 [M+H]⁺, RT 2.73 min.

### Example 6

### Preparation of 8-chloro-6-fluoro-4-oxo-7-(4-pyrimidin-2-ylpiperazin-1-yl)-1-[4-(pyrrolidin-1-ylmethyl)phenyl]-1,4-dihydroquinoline-3-carboxylic acid

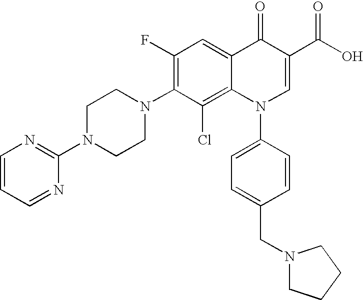

Example 6 was prepared using a similar protocol as Example 1, using 2-piperazin-1-ylpyrimidine in step 1. ¹H NMR (DMSO): δ 11.2 (s, 1H), 8.6 (s, 1H), 8.4 (d, 2H), 8.1 (d, 1H), 7.8 (d, 2H), 7.7 (d, 2H), 6.6 (t, 1H), 4.45 (d, 2H), 3.8 (s, 4H), 3.4 (m, 2H), 3.3 (s, 4H), 3.1 (m, 2H), 2.04 (m, 2H), 1.9 (m, 2H). LC-MS: 563 [M+H]⁺, RT 2.35 min.

### Example 7

### Preparation of 8-chloro-7-[4-(3-cyanopyridin-2-yl)piperazin-1-yl]-6-fluoro-4-oxo-1-[4-(pyrrolidin-1-ylmethyl)phenyl]-1,4-dihydroquinoline-3-carboxylic acid

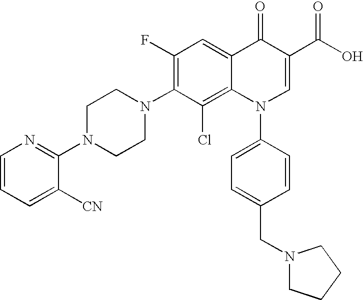

Example 7 was prepared using the procedure as described for Example 1, using 2-piperazin-1-ylnicotinonitrile in step 1. LC-MS: 587 [M+H]⁺, RT 2.47 min.

### Example 8

### Preparation of 8-chloro-7-[4-(2,4-difluorophenyl)piperazin-1-yl]-6-fluoro-4-oxo-1-[4-(pyrrolidin-1-ylmethyl)phenyl]-1,4-dihydroquinoline-3-carboxylic acid

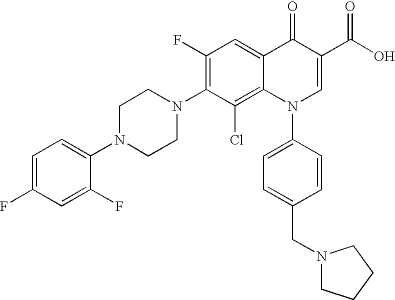

Example 8 was prepared using the procedure as described for Example 1, using 1-(2,4-difluorophenyl)piperazine in step 1. LC-MS: 597 [M+H]⁺, RT 2.76 min.

### Example 9

### Preparation of 7-{4-[3-(aminocarbonyl)pyridin-2-yl]piperazin-1-yl}-8-chloro-6-fluoro-4-oxo-1-[4-(pyrrolidin-1-ylmethyl)phenyl]-1,4-dihydroquinoline-3-carboxylic acid

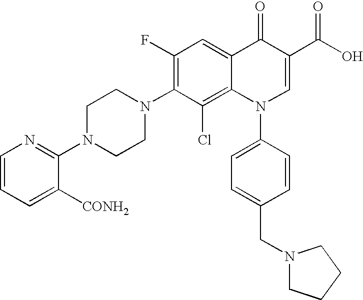

Example 9 was prepared using the procedure as described for Example 1, using in step 1. LC-MS: 605.2 [M+H]⁺, RT 1.99 min.

### Example 10

### Preparation of 7-{4-[2-(aminocarbonyl)phenyl]piperazin-1-yl}-8-chloro-6-fluoro-4-oxo-1-[4-(pyrrolidin-1-ylmethyl)phenyl]-1,4-dihydroquinoline-3-carboxylic acid

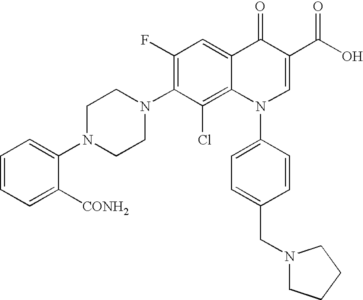

The example was prepared using the procedure as described for Example 1, using in step 1. LC-MS: 604.3 [M+H]⁺, RT 2.21 min.

### Example 11

### Preparation of 8-chloro-6-fluoro-7-[4-(2-methylphenyl)-piperazin-1-yl]-4-oxo-1-[4-(pyrrolidin-1-ylmethyl)phenyl]-1,4-dihydroquinoline-3-carboxylic acid

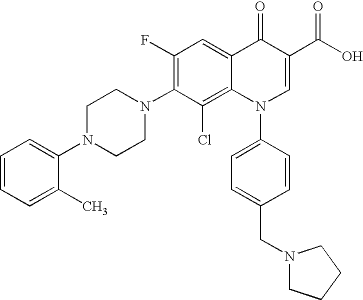

The example was prepared using the procedure as described for Example 1, using 1-(2-methylphenyl)piperazine in step 1. LC-MS: 575 [M+H]⁺, RT 2.82 min.

### Example 12

### Preparation of 8-chloro-7-[4-(4-ethoxyphenyl)piperazin-1-yl]-6-fluoro-4-oxo-1-[4-(pyrrolidin-1-ylmethyl)phenyl]-1,4-dihydroquinoline-3-carboxylic acid

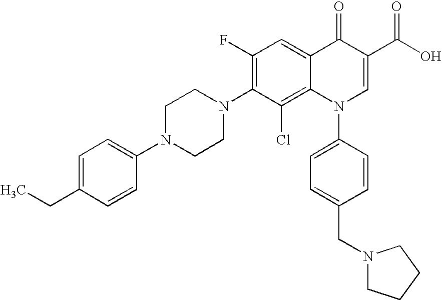

The example was prepared using the procedure as described for Example 1, using 1-(4-ethoxyphenyl)piperazine in step 1. LC-MS: 605 [M+H]⁺, RT 2.37 min.

### Example 13

### Preparation of 8-chloro-6-fluoro-7-[4-(6-methylpyridin-2-yl)-piperazin-1-yl]-4-oxo-1-[4-(pyrrolidin-1-ylmethyl)phenyl]-1,4-dihydroquinoline-3-carboxylic acid

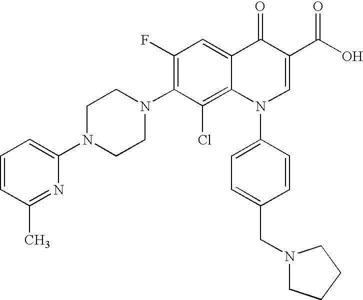

The example was prepared using the procedure as described for Example 1, using 1-(3-methylpyridin-2-yl)piperazine in step 1. LC-MS: 576 [M+H]⁺, RT 1.85 min.

### Example 14

### Preparation of 8-chloro-6-fluoro-4-oxo-1-[4-(pyrrolidin-1-yl-methyl)phenyl]-7-{4-[3-(trifluoromethyl)pyridin-2-yl]piperazin-1-yl}-1,4-dihydroquinoline-3-carboxylic acid

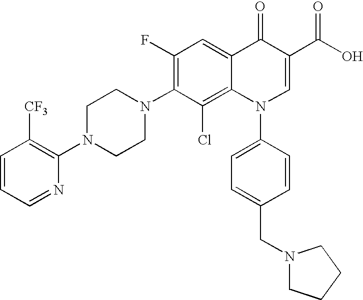

The example was prepared using the procedure as described for Example 1, using in step 1. LC-MS: 630 [M+H]⁺, RT 3.28 min.

### Example 15

### Preparation of 8-chloro-7-[4-(2-cyanophenyl)piperazin-1-yl]-6-fluoro-4-oxo-1-[4-(pyrrolidin-1-ylmethyl)phenyl]-1,4-dihydroquinoline-3-carboxylic acid

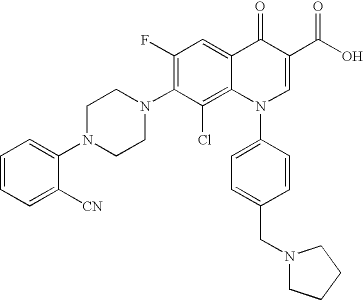

The example was prepared using the procedure as described for Example 1, using 2-piperazin-1-ylbenzonitrile in step 1. LC-MS: [M+H]⁺, RT min.

### Example 16

### Preparation of 8-chloro-7-[4-(2-ethoxyphenyl)piperazin-1-yl]-6-fluoro-4-oxo-1-[4-(pyrrolidin-1-ylmethyl)phenyl]-1,4-dihydroquinoline-3-carboxylic acid

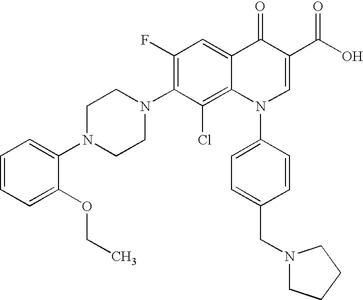

The example was prepared using the procedure as described for Example 1, using 1-(2-ethoxyphenyl)piperazine in step 1. LC-MS: 607 [M+H]⁺, RT 2.49 min.

### Example 17

### Preparation of 8-chloro-6-fluoro-1-{4-[(2-methylpyrrolidin-1-yl)methyl]phenyl}-4-oxo-7-(4-pyridin-2-ylpiperazin-1-yl)-1,4-dihydroquinoline-3-carboxylic acid

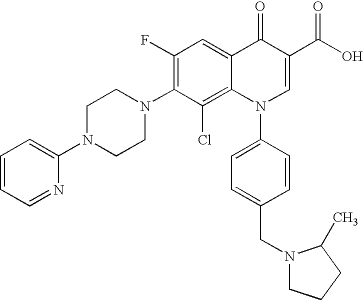

The example was prepared using the procedure as described for the preparation of Example 27. 2-Methylpyrrolidine was used in step 2 instead of (3S)-pyrrolidin-3-ol and using 1-pyridin-2-ylpiperazine instead of 2-piperazin-1-ylpyrimidine in step 3. LC-MS: 576.2 [M+H]⁺, RT 2.39 min.

### Example 18

### Preparation of 8-chloro-6-fluoro-7-[4-(4-fluorophenyl)-piperazin-1-yl]-1-{4-[(2-methylpyrrolidin-1-yl)methyl]phenyl}-4-oxo-1,4-dihydroquinoline-3-carboxylic acid

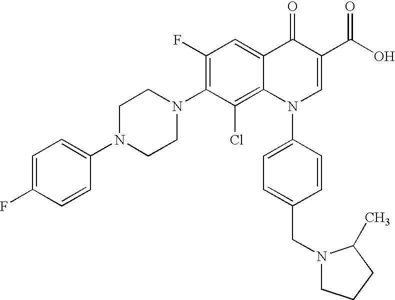

The example was prepared using the procedure as described for the preparation of Example 27. 2-Methylpyrrolidine was used in step 2 instead of (3S)-pyrrolidin-3-ol and using 1-(4-fluorophenyl)piperazine instead of 2-piperazin-1-ylpyrimidine in step 3. LC-MS: 593.2 [M+H]⁺, RT 3.17 min.

### Example 19

### Preparation of 8-chloro-1-{4-[(2,5-dimethylpyrrolidin-1-yl)methyl]phenyl}-6-fluoro-4-oxo-7-(4-pyridin-2-ylpiperazin-1-yl)-1,4-dihydroquinoline-3-carboxylic acid

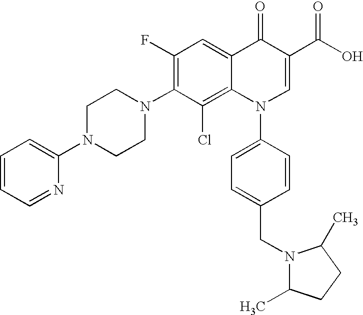

The example was prepared using the procedure as described for the preparation of Example 27. 2,5-Dimethylpyrrolidine was used in step 2 instead of (3S)-pyrrolidin-3-ol and using 1-pyridin-2-ylpiperazine instead of 2-piperazin-1-ylpyrimidine in step 3. LC-MS: 590.6 [M+H]⁺, RT 1.97 min.

### Example 20

### Preparation of 8-chloro-1-{4-[(2,5-dimethyl-2,5-dihydro-1H-pyrrol-1-yl)methyl]phenyl}-6-fluoro-4-oxo-7-(4-pyrimidin-2-ylpiperazin-1-yl)-1,4-dihydroquinoline-3-carboxylic acid

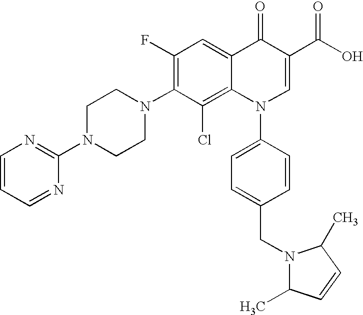

The example was prepared using the procedure as described for the preparation of Example 27. 2,5-Dimethyl-2,5-dihydro-1H-pyrrole was used in step 2 instead of (3S)-pyrrolidin-3-ol. LC-MS: 589.2 [M+H]⁺, RT 2.94 min.

### Example 21

### Preparation of 8-chloro-6-fluoro-1-(4-{[(2R)-2-(methoxy-methyl)pyrrolidin-1-yl]methyl}phenyl)-4-oxo-7-(4-pyridin-2-ylpiperazin-1-yl)-1,4-dihydroquinoline-3-carboxylic acid

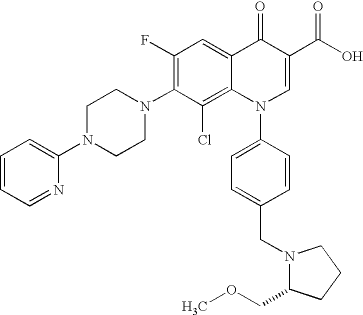

The example was prepared using the procedure as described for the preparation of Example 27. (2R)-2-(methoxymethyl)pyrrolidine was used in step 2 instead of (3S)-pyrrolidin-3-ol and using 1-pyridin-2-ylpiperazine instead of 2-piperazin-1-ylpyrimidine in step 3. LC-MS: 606 [M+H]⁺, RT 1.90 min.

### Example 22

### Preparation of 8-chloro-6-fluoro-1-(4-{[(2R)-2-(methoxy-methyl)pyrrolidin-1-yl]methyl}phenyl)-4-oxo-7-(4-pyrimidin-2-ylpiperazin-1-yl)-1,4-dihydroquinoline-3-carboxylic acid

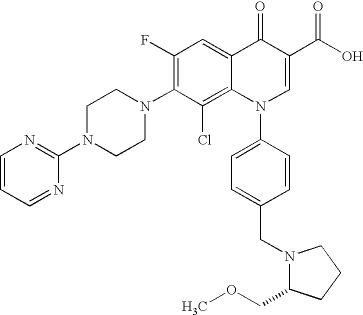

The example was prepared using the procedure as described for the preparation of Example 27. (2R)-2-(methoxymethyl)pyrrolidine was used in step 2 instead of (3S)-pyrrolidin-3-ol. LC-MS: 607 [M+H]⁺, [M+H]⁺, RT 2.42 min.

### Example 23

### Preparation of 8-chloro-6-fluoro-7-[4-(4-fluorophenyl)-piperazin-1-yl]-1-(4-{[(2R)-2-(methoxymethyl)pyrrolidin-1-yl]methyl}phenyl)-4-oxo-1,4-dihydroquinoline-3-carboxylic acid

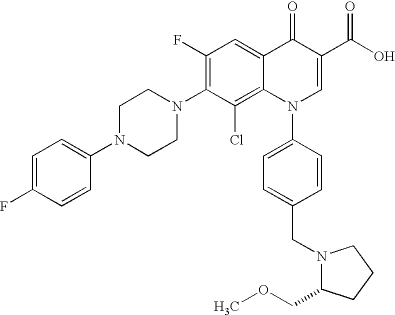

The example was prepared using the procedure as described for the preparation of Example 27. (2R)-2-(methoxymethyl)pyrrolidine was used in step 2 instead of (3S)-pyrrolidin-3-ol and using 1-(4-fluorophenyl)piperazine instead of 2-piperazin-1-ylpyrimidine in step 3. LC-MS: 623 [M+H]⁺, RT 2.69 min.

### Example 24

### Preparation of 8-chloro-6-fluoro-1-(4-{[(2S)-2-(methoxy-methyl)pyrrolidin-1-yl]methyl}phenyl)-4-oxo-7-(4-pyridin-2-ylpiperazin-1-yl)-1,4-dihydroquinoline-3-carboxylic acid

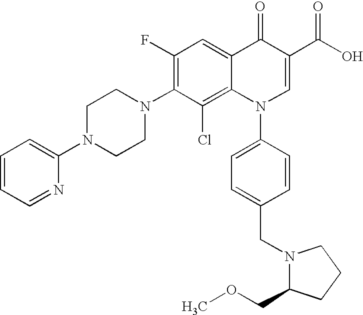

The example was prepared using the procedure as described for the preparation of Example 27. (2S)-2-(methoxymethyl)pyrrolidine was used in step 2 instead of (3S)-pyrrolidin-3-ol and using 1-pyridin-2-ylpiperazine instead of 2-piperazin-1-ylpyrimidine in step 3. LC-MS: 606 [M+H]⁺, RT 1.99 min.

### Example 25

### Preparation of 8-chloro-6-fluoro-7-[4-(4-fluorophenyl)-piperazin-1-yl]-1-(4-{[(2S)-2-(methoxymethyl)pyrrolidin-1-yl]methyl}phenyl)-4-oxo-1,4-dihydroquinoline-3-carboxylic acid

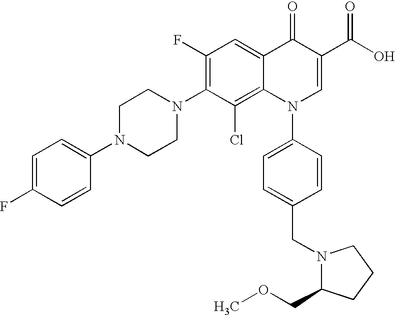

The example was prepared using the procedure as described for the preparation of Example 27. (2R)-2-(methoxymethyl)pyrrolidine was used in step 2 instead of (3S)-pyrrolidin-3-ol and using 1-(4-fluorophenyl)piperazine instead of 2-piperazin-1-ylpyrimidine in step 3. LC-MS: 623 [M+H]⁺, RT 3.19 min.

### Example 26

### Preparation of 8-chloro-6-fluoro-1-(4-{[(2S)-2-(methoxy-methyl)pyrrolidin-1-yl]methyl}phenyl)-4-oxo-7-(4-pyrimidin-2-ylpiperazin-1-yl)-1,4-dihydroquinoline-3-carboxylic acid

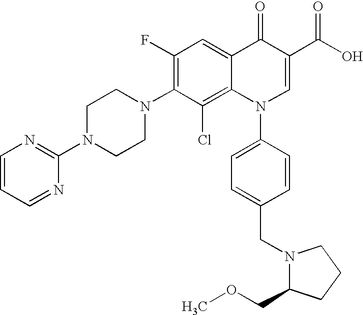

The example was prepared using the procedure as described for the preparation of Example 27. (2R)-2-(methoxymethyl)pyrrolidine was used in step 2 instead of (3S)-pyrrolidin-3-ol. LC-MS: 607 [M+H]⁺, RT 2.41 min.

### Example 27

### Preparation of 8-chloro-6-fluoro-1-(4-{[(3S)-3-hydroxy-pyrrolidin-1-yl]methyl}phenyl)-4-oxo-7-(4-pyrimidin-2-ylpiperazin-1-yl)-1,4-dihydroquinoline-3-carboxylic acid

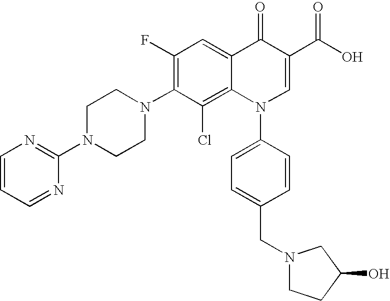

### Step 1: Preparation of ethyl 8-chloro-6-fluoro-1-[4-(hydroxy-methyl)phenyl]-4-oxo-7-(4-pyridin-2-ylpiperazin-1-yl)-1,4-dihydroquinoline-3-carboxylate

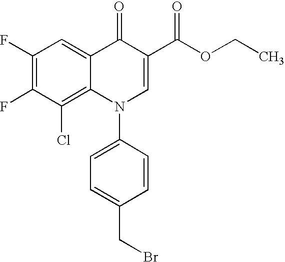

A solution of ethyl 8-chloro-6,7-difluoro-1-[4-(hydroxymethyl)phenyl]-4-oxo-1,4-dihydroquinoline-3-carboxylate (Intermediate F, 3.2 g, 8.2 mmol) in ether/CH₂Cl₂ (30 mL, 1:1) was treated with PBr₃ (4.9 mmol, 4.9 mL of 1N solution in CH₂Cl₂) at 0° C., and stirred at rt for 10 min. The reaction mixture was then poured into a mixture of ether and ice water. The organic layer washed with water, brine, dried (Na₂SO₄), and concentrated. The desired product was then isolated by recrystallization from ethyl acetate/hexane as a light yellow powder (2.95 g, 78%). ¹H NMR (DMSO-d₆): δ 8.39 (s, 1H); 8.21 (t, 1H); 7.64 (m, 4H); 4.80 (s, 2H); 4.23 (q, 2H); 1.26 (t, 3H). MS [M+H]⁺: 456.4 m/z. Calcd 455. RT (LC-MS): 3.33 min. TLC (CH₂Cl₂/2M NH₃ in MeOH 95/5) R_{f}=0.53

### Step 2: Preparation of ethyl 8-chloro-6,7-difluoro-1-(4-{[(3S)-3-hydroxypyrrolidin-1-yl]methyl}phenyl)-4-oxo-1,4-dihydroquinoline-3-carboxylate

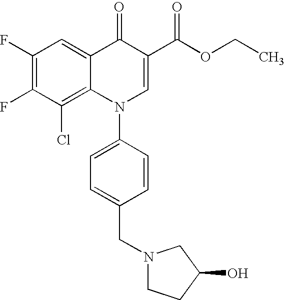

To a solution of ethyl 8-chloro-6-fluoro-1-[4-(hydroxymethyl)phenyl]-4-oxo-7-(4-pyridin-2-ylpiperazin-1-yl)-1,4-dihydroquinoline-3-carboxylate (500 mg, 1.09 mmol) in CH₂Cl₂ was added (3S)-pyrrolidin-3-ol (190 mg, 2.19 mmol) at 0° C. and the mixture was stirred at rt overnight. The reaction mixture was diluted with CH₂Cl₂ and washed with brine, then water. The organic layer was dried and then concentrated. The crude product was purified by silica gel column (CH₂Cl₂ with 1% to 5% of 2M NH₃ in MeOH) to give the desired product as a white powder (376 mg, 74%). ¹H NMR (DMSO-d₆): δ 8.37 (s, 1H); 8.21 (t, 1H); 7.54 (d, J=8.4, 2H); 7.46 (d, J=8.4, 2H); 4.70 (d, J=4.4, 1H); 4.23 (m, 3H); 3.69 (q, 2H); 2.71 (m, 1H); 2.60(m, 1H); 2.46(m, 1H); 2.34(m, 1H); 2.03(m, 1H); 1.57(m, 1H); 1.26 (t, 3H). MS [M+H]⁺: 463.1 m/z. Calcd 462. RT (LC-MS): 2.44 min. TLC (CH₂Cl₂/2M NH₃ in MeOH 95/5) R_{f}=0.22

### Step 3: Preparation of ethyl 8-chloro-6-fluoro-1-(4-{[(3S)-3-hydroxypyrrolidin-1yl]methyl}phenyl)-4-oxo-7-(4-pyridin-2-ylpiperazin-1-yl)-1,4-dihydroquinoline-3-carboxylate

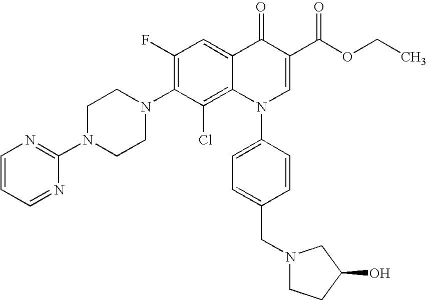

The solution of ethyl 8-chloro-6,7-difluoro-1-(4-{[(3S)-3-hydroxypyrrolidin-1-yl]methyl}phenyl)-4-oxo-1,4-dihydroquinoline-3-carboxylate (100 mg, 0.22 mmol), 2-(1-piperazinyl)pyrimidine (110 mg, 0.65 mmol) and DIEA (0.28 g, 2.16 mmol) in 2 mL of DMSO was heated at 90° C. for 24 h. The solvent was removed under vacuo, and the crude mixture was purified by HPLC to give the desired product as an off-white powder (103 mg, 63%). ¹H NMR (CD₃OD): δ 8.35 (m, 3H); 7.95 (d, J=12, 1H); 7.47 (m, 4H); 6.64 (t, 1H); 4.70 (d, J=4.8, 1H); 4.23 (m, 3H); 3.79 (s, broad, 4H); 3.67 (q, 2H); 3.18 (s, 4H); 2.71(m, 1H); 2.63(m, 1H); 2.45(m, 1H); 2.35 (m, 1H); 2.03 (m, 1H); 1.58 (m, 1H); 1.26 (t, 3H). MS [M+H]⁺: 607.3 m/z. Calcd 606. RT (LC-MS): 2.70 min.

### Step 4: Preparation of the Title Compound

The solution of ethyl 8-chloro-6-fluoro-1-(4-{[(3S)-3-hydroxypyrrolidin-1yl]methyl}phenyl)-4-oxo-7-(4-pyridin-2-ylpiperazin-1-yl)-1,4-dihydroquinoline-3-carboxylate (30 mg, 0.05 mmol) in iPrOH/HCl/H₂O (2 ml/0.5 mL/0.5 mL) was heated at 95° C. overnight. The reaction mixture was cooled to rt and the solvent was removed. Cold 2-propanol was added. The precipitates formed was filtered and washed with 2-propanol/hexane to give the desired product as a light yellow powder (18 mg, 56%). ¹H NMR (DMSO-d₆): δ 11.20 (broad s, 1H), 8.58 (m, 1H); 8.37 (d, J=4.4, 2H); 8.13 (d, J=11.6, 1H); 7.84 (m, 4H); 6.67 (t, 1H); 4.50 (m, 4H); 3.83 (s, broad 4H); 3.57 (m, 1H); 3.43 (m, 1H); 3.26 (m, 6H); 2.99 (m, 0.5H); 2.31 (m, 0.5H); 2.04 (m, 1H). MS [M+H]⁺: 579.2 m/z. Calcd 578. RT (LC-MS): 2.80 min.

### Example 28

### Preparation of 8-chloro-6-fluoro-1-(4-{[(3S)-3-hydroxypyrrolidin-1-yl]methyl}phenyl)-4-oxo-7-(4-pyridin-2-ylpiperazin-1-yl)-1,4-dihydroquinoline-3-carboxylic acid

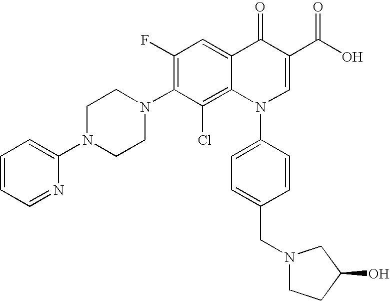

The example was prepared using the procedure as described for the preparation of Example 27. 1-pyridin-2-ylpiperazine was used in step 3 instead of 2-piperazin-1-ylpyrimidine. LC-MS: 578.2 [M+H]⁺, RT 2.22 min.

### Example 29

### Preparation of 8-chloro-6-fluoro-7-[4-(4-fluorophenyl)-piperazin-1-yl]-1-(4-{[(3S)-3-hydroxypyrrolidin-1-yl]methyl}phenyl)-4-oxo-1,4-dihydroquinoline-3-carboxylic acid

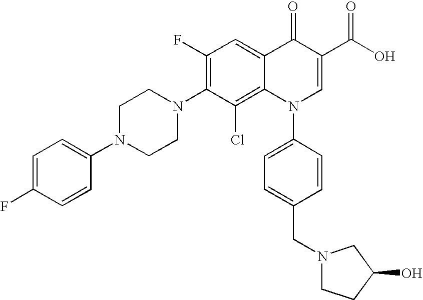

The example was prepared using the procedure as described for the preparation of Example 27. 1-(4-fluorophenyl)piperazine was used in step 3 instead of 2-piperazin-1-ylpyrimidine. LC-MS: 595.2 [M+H]⁺, RT 2.56 min.

### Example 30

### Preparation of 8-chloro-7-[4-(2-cyanophenyl)piperazin-1-yl]-6-fluoro-1-(4-{[(3S)-3-hydroxypyrrolidin-1-yl]methyl}phenyl)-4-oxo-1,4-dihydroquinoline-3-carboxylic acid

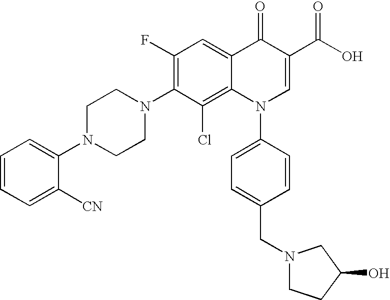

The example was prepared using the procedure as described for the preparation of Example 27. 2-piperazin-1-ylbenzonitrile was used in step 3 instead of 2-piperazin-1-ylpyrimidine. LC-MS: 602.3 [M+H]⁺, RT 2.59 min.

### Example 31

### Preparation of 8-chloro-6-fluoro-7-[4-(4-fluorophenyl)-piperazin-1-yl]-1-(4-{[(3R)-3-hydroxypyrrolidin-1-yl]methyl}phenyl)-4-oxo-1,4-dihydroquinoline-3-carboxylic acid

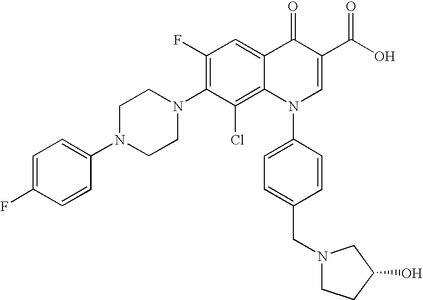

The example was prepared using the procedure as described for the preparation of Example 27. (3R)-Pyrrolidin-3-ol was used in step 2 instead of (3S)-pyrrolidin-3-ol and 1-(4-fluorophenyl)piperazine was used in step 3 instead of 2-piperazin-1-ylpyrimidine. LC-MS: 595.2 [M+H]⁺, RT 3.06 min.

### Example 32

### Preparation of 8-chloro-7-[4-(2-cyanophenyl)piperazin-1-yl]-6-fluoro-1-(4-{[(3R)-3-hydroxypyrrolidin-1-yl]methyl}phenyl)-4-oxo-1,4-dihydroquinoline-3-carboxylic acid

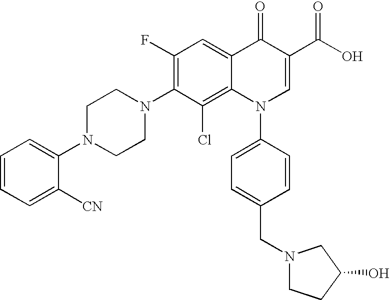

The example was prepared using the procedure as described for the preparation of Example 27. (3R)-Pyrrolidin-3-ol was used in step 2 instead of (3S)-pyrrolidin-3-ol and 2-piperazin-1-ylbenzonitrile was used in step 3 instead of 2-piperazin-1-ylpyrimidine. LC-MS: 602.2 [M+H]⁺, RT 3.11 min.

### Example 33

### Preparation of 8-chloro-6-fluoro-4-oxo-7-(4-pyrimidin-2-ylpiperazin-1-yl)-1-[6-(pyrrolidin-1-ylmethyl)pyridin-3-yl]-1,4-dihydroquinoline-3-carboxylic acid

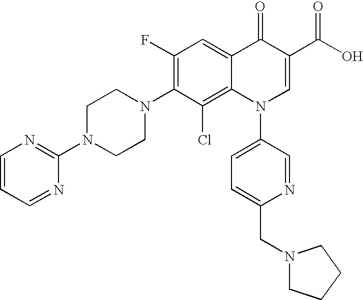

### Step 1: Preparation of 5-nitropyridine-2-carbaldehyde

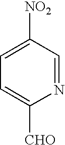

A mixture of 2-methyl-5-nitropyridine (3 g), selenium(IV) oxide (2.9 g), 1,4-dioxane (25 mL) and water (0.5 mL) was refluxed for 4 hrs. The resulting black solid was filtered through Celite® bed and washed with ether. The filtrate was treated with saturate aqueous NaHCO₃ and filtered again. The filtrate was extracted with ether twice and the solvent was concentrated. The residue was purified with a short silica gel column eluted with 20% ethyl acetate in hexane to give 1.0 g of orange precipitate 2 (35% yield). ¹H NMR (CD₂Cl₂): δ 10.15 (s, 1H), 9.55 (s, 1H), 8.7 (d, 1H), 8.15 (s, 1H).

### Step 2: Preparation of 5-nitro-2-(pyrrolidin-1-ylmethyl)pyridine

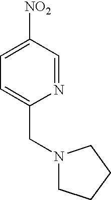

A mixture of 5-nitropyridine-2-carbaldehyde and pyrrolidine in 1,2-dichloroethane (20 mL) was treated with sodium triaectoxyborohydride and acetic acid. The reaction mixture was stirred at room temperature overnight. LC-MS showed major desired product peak at m/z 208 [M+H]⁺. The reaction mixture was quenched by adding 1 N NaOH, diluted with dichloromethane and passed through a Celite® bed. The organic phase was poured to a short silica gel column and eluted with 2% MeOH (containing 1 N NH₃) in dichloromethane to give dark crude product. The solvent was concentrated and the residue was purified with a short column eluted with 20% ethyl acetate in hexanes to give 1.0 g of desired product as a orange precipitate (73% yield).

### Step 3: Preparation of 6-(pyrrolidin-1-ylmethyl)pyridin-3-amine

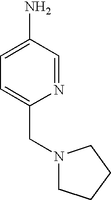

To a solution of 5-nitro-2-(pyrrolidin-1-ylmethyl)pyridine (0.5 g) in ethyl acetate (10 mL) and methanol (10 mL) was added Raney-Ni under nitrogen atmosphere. The solution was purged with hydrogen using a balloon and checked by TLC. After 1 hr reaction, TLC (19:1, CH₂Cl₂-MeOH with 2N NH₃, 19:1) showed all starting material had been consumed and two slow moving spots appeared. Hydrogenation was continued for additional 1 h when TLC showed a single spot. The catalyst was filtered off, and solvent was concentrated to give 0.33 g of desired product as light yellow oil (77% yield).

### Step 4: Preparation of ethyl 8-chloro-6,7-difluoro-4-oxo-1-[6-(pyrrolidin-1-ylmethyl)pyridin-3-yl]-1,4-dihydroquinoline-3-carboxylate

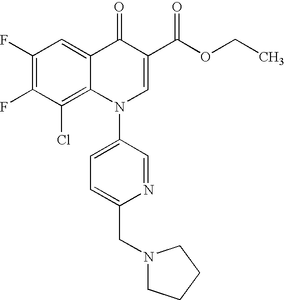

This intermediate was synthesized using the procedure as described for the synthesis of Intermediate B, using 6-(pyrrolidin-1-ylmethyl)pyridin-3-amine (step 3 product) instead of 4-pyrrolidin-1-ylmethyl-phenylamine (71% yield). ¹H NMR (CD₂Cl₂): δ 8.6 (d, 1H), 8.4 (s, 1H), 8.3 (q, 1H), 7.7 (s, 2H), 4.3 (q, 2H), 4.0 (s, 2H), 3.6 (s, 2H), 2.7 (s, 4H), 1.9 (s, 4H), 1.4 (t, 3H).

### Step 5: Preparation of ethyl 8-chloro-6-fluoro-4-oxo-7-(4-pyrimidin-2-ylpiperazin-1-yl)-1-[6-(pyrrolidin-1-ylmethyl)pyridin-3-yl]-1,4-dihydroquinoline-3-carboxylate

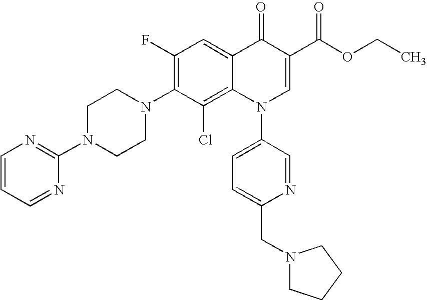

A solution of ethyl 8-chloro-6,7-difluoro-4-oxo-1-[6-(pyrrolidin-1-ylmethyl)pyridin-3-yl]-1,4-dihydroquinoline-3-carboxylate (80 mg), 2-(1-piperazinyl)pyrimidine (50 mg), and DIEA (0.06 mL) in DMSO (1.5 mL) was heated at 90° C. overnight. The heat was removed, and mixture was cooled to room temperature. The solvent was concentrated and residue was purified on a column (10 g silica gel), eluting with MeOH in CH₂Cl₂ from 0 to 10% to give 25 mg of pure precipitate and 36 mg of impure precipitate. R_{f}=0.45 (10% MeOH in CH₂Cl₂). The impure precipitate was purified again on a silica gel column. Total 43 mg of the desired product was obtained (40% yield). ¹H NMR (CD₂Cl₂): δ 8.6 (d, 1H), 8.4 (s, 1H), 8.35 (dd, 1H), 8.3 (d, 1H), 8.1 (d, 2H), 7.8 (s, 1H), 6.5 (t, 1H), 4.4 (q, 2H), 4.3 (s, 2H), 3.9 (wide, 4H), 3.3 (s, 4H), 3.1 (wide, 4H), 2.1 (s, 4H), 1.4 (t, 3H). LC-MS: m/z 592 [M+H]⁺, RT 2.70 min.

### Step 6: Preparation of the Title Compound

A solution of 6 (43 mg) in a mixed solvent (1.5 mL) consisting of IPA, HCl (conc.) and water (100:20:10) was heated at 90° C. overnight. The solvent was concentrated. the resulting precipitate was treated with isopropanol, filtered, washed with isopropanol and dried under high vacuum over night to give 13 mg (26% yield) of 7 (2 HCl salt). ¹H NMR (D₂O): δ 8.9 (m, 2H), 8.5 (s, 2H), 8.1 (m, 2H), 7.9 (m, 1H), 6.9 (s, 1H), 4.8 (s, 2H), 4.0 (s, 2H), 3.9 (s, 2H), 3.8 (s, 4H), 3.6 (s, 4H), 3.4 (s, 2H), 2.3 (s, 2H), 2.17 (s, 2H). LC-MS: 564 [M+H]⁺, RT 2.25 min.

### Example 34

### Preparation of 8-chloro-6-fluoro-4-oxo-7-(4-pyridin-2-yl-piperazin-1-yl)-1-[6-(pyrrolidin-1-ylmethyl)pyridin-3-yl]-1,4-dihydroquinoline-3-carboxylic acid

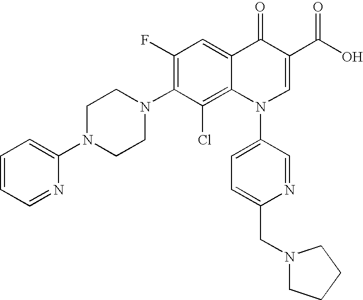

A similar procedure as described in the synthesis of Example 33 was used, except 2-(1-piperazinyl)pyridine was used in place of 2-(1-piperazinyl)pyrimidine in step 4. LC-MS: 563 [M+H]⁺, RT 1.74 min.

### Example 35

### Preparation of 8-chloro-7-[4-(3-cyanopyridin-2-yl)piperazin-1-yl]-6-fluoro-4-oxo-1-[6-(pyrrolidin-1-ylmethyl)pyridin-3-yl]-1,4-dihydroquinoline-3-carboxylic acid

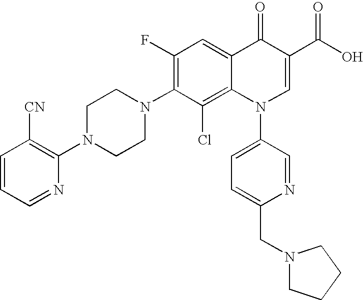

A similar procedure as described in the synthesis of Example 33 was used, except 2-piperazin-1-ylnicotinonitrile was used in place of 2-(1-piperazinyl)pyrimidine in step 4. LC-MS: 588 [M+H]⁺, RT 2.54 min.

### Example 36

### Preparation of 8-chloro-7-[4-(2-cyanophenyl)piperazin-1-yl]-6-fluoro-4-oxo-1-[6-(pyrrolidin-1-ylmethyl)pyridin-3-yl]-1,4-dihydroquinoline-3-carboxylic acid

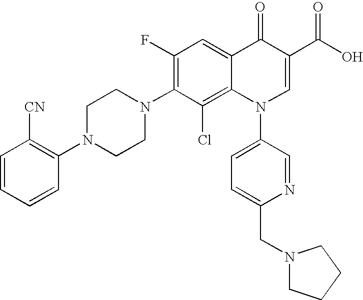

A similar procedure as described in the synthesis of Example 33 was used, except 2-piperazin-1-ylbenzonitrile was used in place of 2-(1-piperazinyl)pyrimidine in step 4. LC-MS: 587 [M+H]⁺, RT 2.66 min.

### Example 37

### Preparation of 8-chloro-6-fluoro-7-[4-(4-fluorophenyl)-piperazin-1-yl]-4-oxo-1-[6-(pyrrolidin-1-ylmethyl)pyridin-3-yl]-1,4-dihydroquinoline-3-carboxylic acid

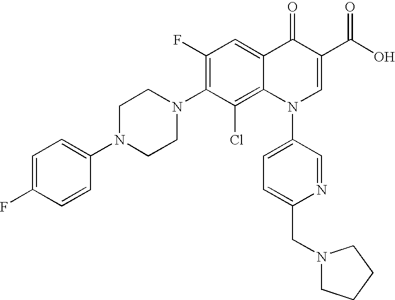

A similar procedure as described in the synthesis of Example 33 was used, except 1-(4-fluorophenyl)piperazine was used in place of 2-(1-piperazinyl)-pyrimidine in step 4. LC-MS: 580 [M+H]⁺, PT 2.59 min.

### Example 38

### Preparation of 8-chloro-7-[4-(4-chlorophenyl)piperazin-1-yl]-6-fluoro-4-oxo-1-[6-(pyrrolidin-1-ylmethyl)pyridin-3-yl]-1,4-dihydroquinoline-3-carboxylic acid

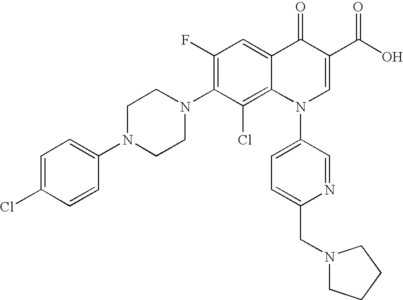

A similar procedure as described in the synthesis of Example 33 was used, except 1-(4-chlorophenyl)piperazine was used in place of 2-(1-piperazinyl)pyrimidine in step 4. LC-MS: 596 [M+H]⁺, RT 2.84 min.

### Example 39

### Preparation of 8-chloro-7-[4-(2-cyanophenyl)piperazin-1-yl]-6-fluoro-1-[2-fluoro-4-(pyrrolidin-1-ylmethyl)phenyl]-4-oxo-1,4-dihydroquinoline-3-carboxylic acid

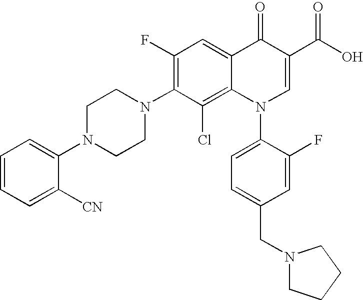

### Step 1: Preparation of 4-(bromomethyl)-2-fluoro-1-nitrobenzene

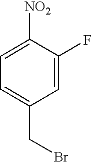

In a 2 L of round bottle flask was placed 3-fluoro-4-nitrotoluene (7.2 g) in 700 mL of dichloromethane. To this was added 500 mL of water and then potassium bromate (31 g), followed by adding sodium hydrosulfite (32 g) from a funnel dropwise as a solution in 200 mL of water during the period of 16 h. The reaction was transferred to a separatory funnel and washed with Na₂S₂O₃ aq NaHCO₃ and water. The organic phase was dried over MgSO₄, filtered and evaporated to produce an oil. The oil was put in the freezer over the weekend to give crude yellow precipitate (10.55 g). The crude product was purified with a column (125 g silica gel), eluting with EtOAc in hexanes (from 0 to 20%) to give 3.2 g of white precipitate (58% yield). ¹H NMR (CD₂Cl₂): δ 8.05(t, 1H), 7.35 (m, 2H), 4.5 (s, 2H).

### Step 2: Preparation of 1-(3-fluoro-4-nitrobenzyl)pyrrolidine

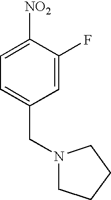

A solution of 4-(bromomethyl)-2-fluoro-1-nitrobenzene (2.4 g) and DIEA (3.6 mL) in THF (80 mL) was slowly added 2.65 g of pyrrolidine in 20 mL of THF in 0° C. The solution was stirred at room temperature for 6 hrs. The precipitate was filtered off and the solvent was concentrated. The residue was purified with a short silica gel column eluted with EtOAc in hexanes (from 20 to 60%) to give 1.8 g of desired product as light yellow oil in 65% yield.

### Step 3: Preparation of 2-fluoro-4-(pyrrolidin-1-ylmethyl)aniline

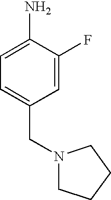

To a solution of 1-(3-fluoro-4-nitrobenzyl)pyrrolidine (1.85 g) in EtOAc (30 mL) was added Raney-Ni under a nitrogen atmosphere. The solution was purged with hydrogen using a balloon and checked by TLC. After 2 h reaction, TLC (CH₂Cl₂-MeOH with 2N NH₃, 19:1) showed all starting material had been consumed. A slow moving spot was observed. The catalyst was filtered off, and solvent was concentrated to give 1.5 g of the desired product as light yellow oil in 93% yield. ¹H NMR (CD₂Cl₂): δ 7.0 (d, 1H), 6.9 (d, 1H), 6.7 (q, 1H), 3.8 (s, 2H), 3.3 (s, 2H), 2.5 (m, 4H), 1.8 (m, 4H).

### Step 4: Preparation of ethyl 8-chloro-6,7-difluoro-1-[2-fluoro-4-(pyrrolidin-1-ylmethyl)phenyl]-4-oxo-1,4-dihydroquinoline-3-carboxylate

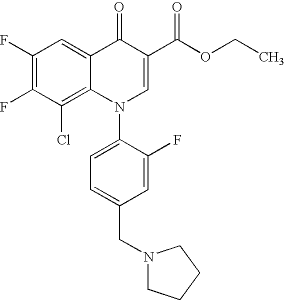

This intermediate was synthesized using the procedure as described for the synthesis of Intermediate B, using 2-fluoro-4-(pyrrolidin-1-ylmethyl)aniline (step 3 product) instead of 4-pyrrolidin-1-ylmethyl-phenylamine (89% yield). ¹H NMR (CD₂Cl₂): δ 8.6 (dd, 1H), 7.4 (m, 1H), 7.2 (m, 2H), 5.33 (s, 1H), 4.1 (q, 2H), 3.6 (s, 2H), 2.5 (s, 4H), 1.8 (s, 4H), 1.1 (t, 3H).

### Step 5: Preparation of ethyl 8-chloro-7-[4-(2-cyanophenyl)piperazin-1-yl]-6-fluoro-1-[2-fluoro-4-(pyrrolidin-1-ylmethyl)phenyl]-4-oxo-1,4-dihydro-quinoline-3-carboxylate

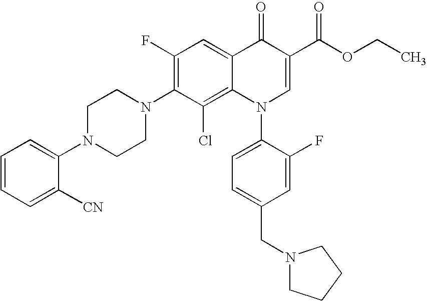

A solution of ethyl 8-chloro-6,7-difluoro-1-[2-fluoro-4-(pyrrolidin-1-ylmethyl)phenyl]-4-oxo-1,4-dihydroquinoline-3-carboxylate (110 mg), 1-(2-cyanophenyl)-piperazine (107 mg), and DIEA (0.1 mL) in DMSO (2.4 mL) was heated at 90° C. overnight. The heat was removed, and mixture was cooled to room temperature. The solvent was concentrated and the residue was purified with a column (10 g silica gel), eluting with MeOH in CH₂Cl₂ (from 0 to 3%) to give 60 mg of the desired product as yellow precipitate in 40% yield. ¹H NMR (CDCl₃): δ 8.4 (s, 1H), 8.2 (s, 1H), 7.6 (d, 1H), 7.5 (m, 2H), 7.4 (s, 1H), 7.0 (m, 2H), 4.4 (s, 2H), 3.9 (wide, 2H), 3.5 (s, 4H), 3.3 (s, 4H), 2.8 (wide, 2H), 2.0 (s, 4H), 1.6 (wide, 2H), 1.4 (t, 3H). LC-MS: 632 m/z [M+H]⁺, RT 2.72 min.

### Step 6: Preparation of the Title Compound

A solution of ethyl 8-chloro-7-[4-(2-cyanophenyl)piperazin-1-yl]-6-fluoro-1-[2-fluoro-4-(pyrrolidin-1-ylmethyl)phenyl]-4-oxo-1,4-dihydroquinoline-3-carboxylate (40 mg) in a mixed solvent (3 mL) consisting of IPA, HCl (conc.) and water (100:20:10). The solution was heated at 90° C. overnight. The solvent was concentrated with to give a precipitate. The precipitate was treated with isopropanol, filtered and washed with isopropanol. The resulting precipitate was dried under high vacuum over night to give 25 mg of pure product as a yellow precipitate (2HCl salt) in 54% yield. ¹H NMR (DMSO): δ 11.0 (s, 1H), 8.7 (s, 1H), 8.2 (d, 1H), 7.8 (d, 2H), 7.7 (d, 1H), 7.6 (m, 1H), 7.2 (d, 1H), 7.1 (t, 1H), 4.5 (s, 2H), 3.4 (s, 6H), 3.3 (s, 4H), 3.1 (s, 2H), 2.1 (s, 2H), 1.9 (m, 2H). LC-MS: 604 m/z [M+H]⁺, RT 3.21 min.

### Example 40

### Preparation of 8-chloro-6-fluoro-1-[2-fluoro-4-(pyrrolidin-1-ylmethyl)phenyl]-4-oxo-7-(4-pyrimidin-2-ylpiperazin-1-yl)-1,4-dihydroquinoline-3-carboxylic acid

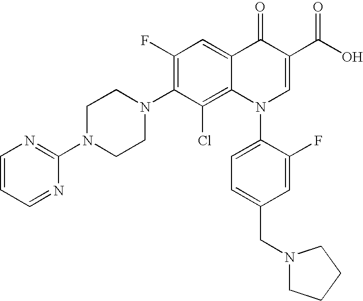

A similar procedure as described in the synthesis of Example 39 was used, except 2-piperazin-1-ylpyrimidine was used in place of 1-(2-cyanophenyl)-piperazine in step 5. LC-MS: 581 [M+H]⁺, RT 2.94 min.

### Example 41

### Preparation of 8-chloro-6-fluoro-7-[4-(4-fluorophenyl)-piperazin-1-yl]-1-[2-fluoro-4-(pyrrolidin-1-ylmethyl)phenyl]-4-oxo-1,4-dihydroquinoline-3-carboxylic acid

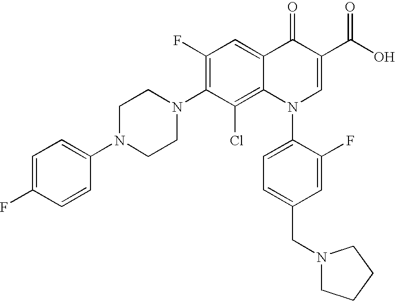

A similar procedure as described in the synthesis of Example 39 was used, except 1-(4-fluorophenyl)piperazine was used in place of 1-(2-cyanophenyl)-piperazine in step 5. LC-MS: 597 [M+H]⁺, RT 3.17 min.

### Example 42

### Preparation of 8-chloro-7-[4-(4-chlorophenyl)piperazin-1-yl]-6-fluoro-1-[2-fluoro-4-(pyrrolidin-1-ylmethyl)phenyl]-4-oxo-1,4-dihydroquinoline-3-carboxylic acid

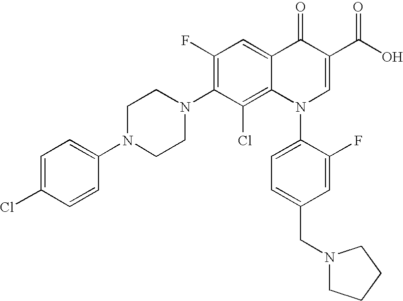

A similar procedure as described in the synthesis of Example 39 was used, except 1-(4-chlorophenyl)piperazine was used in place of 1-(2-cyanophenyl)-piperazine in step 5. LC-MS: 613 [M+H]⁺, RT 3.39 min.

### Example 43

### Preparation of 8-chloro-6-fluoro-1-[2-fluoro-4-(pyrrolidin-1-ylmethyl)phenyl]-4-oxo-7-(4-phenylpiperazin-1-yl)-1,4-dihydroquinoline-3-carboxylic acid

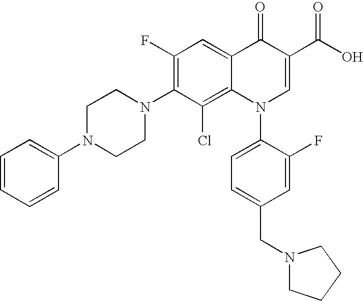

A similar procedure as described in the synthesis of Example 39 was used, except 1-phenylpiperazine was used in place of 1-(2-cyanophenyl)-piperazine in step 5. LC-MS: 579 [M+H]⁺, RT 3.09 min.

### Example 44

### Preparation of 8-chloro-6-fluoro-1-[2-fluoro-4-(pyrrolidin-1-ylmethyl)phenyl]-7-[4-(3-methylpyridin-2-yl)piperazin-1-yl]-4-oxo-1,4-dihydroquinoline-3-carboxylic acid

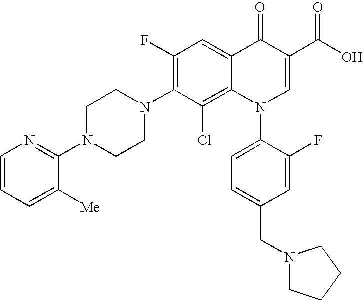

A similar procedure as described in the synthesis of Example 39 was used, except 1-(3-methylpyridin-2-yl)piperazine was used in place of 1-(2-cyanophenyl)-piperazine in step 5. LC-MS: 594 [M+H]⁺, RT 2.19 min.

### Example 45

### Preparation of 8-chloro-7-[4-(3-cyanopyridin-2-yl)piperazin-1-yl]-6-fluoro-1-[2-fluoro-4-(pyrrolidin-1-ylmethyl)phenyl]-4-oxo-1,4-dihydroquinoline-3-carboxylic acid

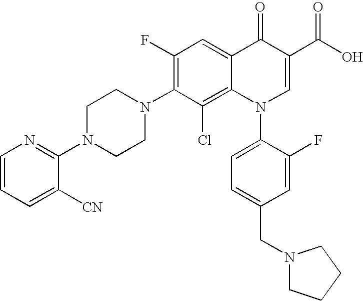

A similar procedure as described in the synthesis of Example 39 was used, except 2-piperazin-1-ylnicotinonitrile was used in place of 1-(2-cyanophenyl)-piperazine in step 5. LC-MS: 605 [M+H]⁺, RT 2.67 min.

### Example 46

### Preparation of 8-chloro-6-fluoro-1-[2-fluoro-4-(pyrrolidin-1-ylmethyl)phenyl]-4-oxo-7-(4-pyridin-2-ylpiperazin-1-yl)-1,4-dihydroquinoline-3-carboxylic acid

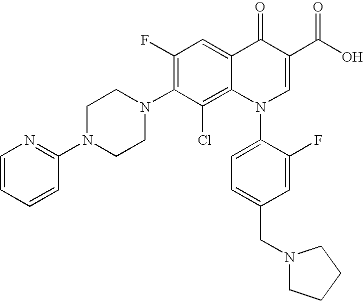

A similar procedure as described in the synthesis of Example 39 was used, except 1-pyridin-2-ylpiperazine was used in place of 1-(2-cyanophenyl)-piperazine in step 5. LC-MS: 580 [M+H]⁺, RT 1.99 min.

### Example 47

### Preparation of 8-chloro-7-[4-(5-cyanopyridin-2-yl)piperazin-1-yl]-6-fluoro-1-[2-fluoro-4-(pyrrolidin-1-ylmethyl)phenyl]-4-oxo-1,4-dihydroquinoline-3-carboxylic acid

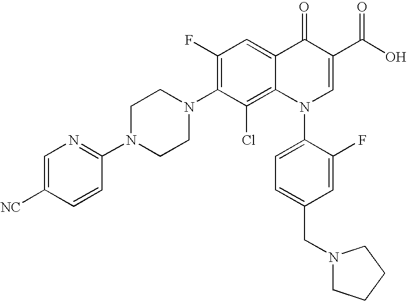

A similar procedure as described in the synthesis of Example 39 was used, except 6-piperazin-1-ylnicotinonitrile was used in place of 1-(2-cyanophenyl)-piperazine in step 5. LC-MS: 605 [M+H]⁺, RT 2.66 min.

### Example 48

### Preparation of 8-chloro-7-[4-(4-cyanophenyl)piperazin-1-yl]-6-fluoro-1-[2-fluoro-4-(pyrrolidin-1-ylmethyl)phenyl]-4-oxo-1,4-dihydroquinoline-3-carboxylic acid

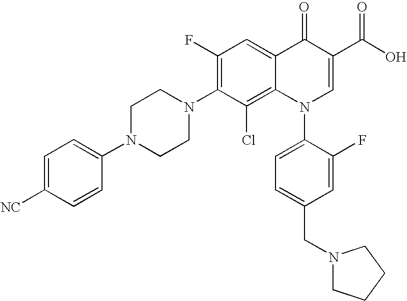

A similar procedure as described in the synthesis of Example 39 was uses, except 4-piperazin-1-ylbenzonitrile was used in place of 1-(2-cyanophenyl)-piperazine in step 5. LC-MS: 604 [M+H]⁺, RT 2.76 min.

### Example 49

### Preparation of 8-chloro-6-fluoro-1-(4-{[methoxy(methyl)-amino]methyl}phenyl)-4-oxo-7-(4-pyridin-2-ylpiperazin-1-yl)-1,4-dihydroquinoline-3-carboxylic acid

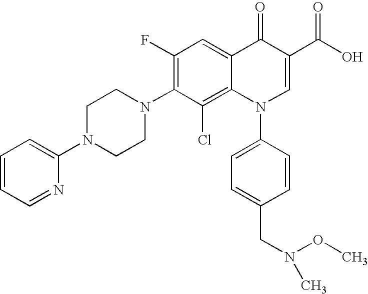

### Step 1: Preparation of (4-{[methoxy(methyl)amino]methyl}phenyl)amine

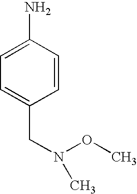

The aniline was prepared using the protocol as described for synthesis of Intermediate A except N,O-dimethyl-hydroxylamine HCl salt was used in step 1 instead of pyrrolidine.

### Step 2: Preparation of the Title Compound

The example was prepared using the procedure as described for synthesis of Example 39 except (4-{[methoxy(methyl)amino]methyl}phenyl)amine was used instead of 2-fluoro-4-(pyrrolidin-1-ylmethyl)aniline in step 4 and 1-pyridin-2-ylpiperazine was used instead of 1-(2-cyanophenyl)-piperazine in step 5. LC-MS: 552 [M+H]⁺, RT 2.41 min.

### Example 50

### Preparation of 8-chloro-6-fluoro-1-(4-{[methoxy(methyl)-amino]methyl}phenyl)-4-oxo-7-(4-pyrimidin-2-ylpiperazin-1-yl)-1,4-dihydroquinoline-3-carboxylic acid

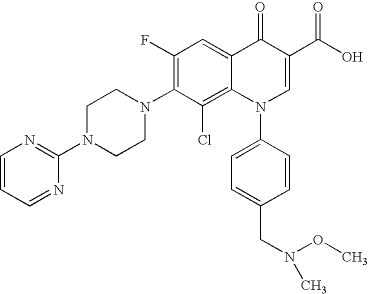

A similar procedure as described in the synthesis of Example 49 was used, except 2-piperazin-1-ylpyrimidine was used in place of 1-pyridin-2-ylpiperazine in step 5. LC-MS: 553 [M+H]⁺, RT 3.37 min.

### Example 51

### Preparation of 8-chloro-6-fluoro-7-[4-(4-fluorophenyl)-piperazin-1-yl]-1-(4-{[methoxy(methyl)amino]methyl}phenyl)-4-oxo-1,4-dihydroquinoline-3-carboxylic acid

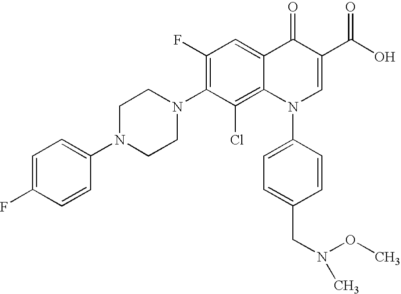

A similar procedure as described in the synthesis of Example 49 was used, except 1-(4-fluorophenyl)piperazine was used in place of 1-pyridin-2-ylpiperazine in step 5. LC-MS: 569 [M+H]⁺, RT 3.77 min.

### Example 52

### Preparation of 8-chloro-7-[4-(4-chlorophenyl)piperazin-1-yl]-6-fluoro-1-(4-{[methoxy(methyl)amino]methyl}phenyl)-4-oxo-1,4-dihydroquinoline-3-carboxylic acid

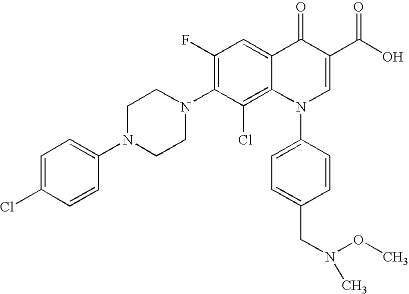

A similar procedure as described in the synthesis of Example 49 was used, except 1-(4-chlorophenyl)piperazine was used in place of 1-pyridin-2-ylpiperazine in step 5. LC-MS: 585 [M+H]⁺, RT 4.15 min.

### Example 53

### Preparation of 8-chloro-7-[4-(2-cyanophenyl)piperazin-1-yl]-6-fluoro-1-(4-{[methoxy(methyl)amino]methyl}phenyl)-4-oxo-1,4-dihydroquinoline-3-carboxylic acid

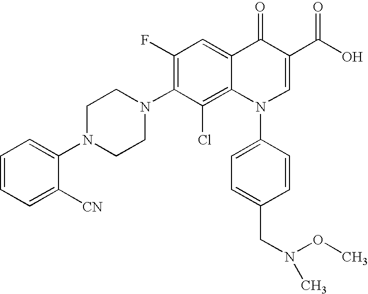

A similar procedure as described in the synthesis of Example 49 was used, except 2-piperazin-1-ylbenzonitrile was used in place of 1-pyridin-2-ylpiperazine in step 5. LC-MS: 576 [M+H]⁺, RT 3.78 min.

### Example 54

### Preparation of 8-chloro-7-[4-(3-cyanopyridin-2-yl)piperazin-1-yl]-6-fluoro-1-(4-{[methoxy(methyl)amino]methyl}phenyl)-4-oxo-1,4-dihydroquinoline-3-carboxylic acid

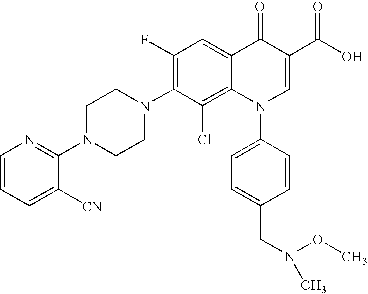

A similar procedure as described in the synthesis of Example 49 was used, except 2-piperazin-1-ylnicotinonitrile was used in place of 1-pyridin-2-ylpiperazine in step 5. LC-MS: 577 [M+H]⁺, RT 3.60 min.

### Example 55

### Preparation of 8-chloro-6-fluoro-1-(4-{[(2-methoxyethyl)-15 (methyl)amino]methyl}phenyl)-4-oxo-7-(4-pyridin-2-ylpiperazin-1-yl)-(1,4-dihydroquinoline-3-carboxylic acid

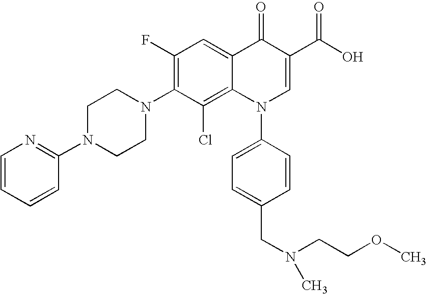

### Step 1: Preparation of 4-{[(2-methoxyethyl)(methyl)amino]methyl}aniline

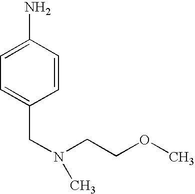

The aniline was prepared using the protocol as described for synthesis of Intermediate A except 2-methoxy-N-methylethanamine was used in step 1 instead of pyrrolidine.

### Step 2: Preparation of the Title Compound

The example was prepared using the procedure as described for the synthesis of Example 39 except 4-{[(2-methoxyethyl)(methyl)-amino]methyl}aniline was used instead of 2-fluoro-4-(pyrrolidin-1-ylmethyl)aniline in step 4 and 1-pyridin-2-ylpiperazine was used instead of 1-(2-cyanophenyl)-piperazine in step 5. LC-MS: 580.2 [M+H]⁺, RT 1.96 min.

### Example 56

### Preparation of 8-chloro-1-{4-[(cyclopentylamino)-methyl]phenyl}-6-fluoro-4-oxo-7-(4-pyridin-2-ylpiperazin-1-yl)-1,4-dihydroquinoline-3-carboxylic acid

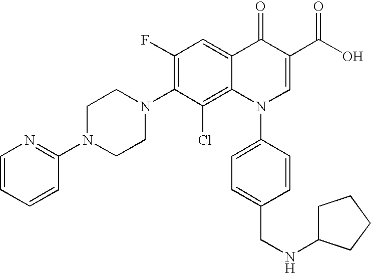

### Step 1: Preparation of ethyl 8-chloro-6-fluoro-1-[4-(hydroxymethyl)phenyl]-4-oxo-7-(4-pyridin-2-ylpiperazin-1-yl)-1,4-dihydro-quinoline-3-carboxylate

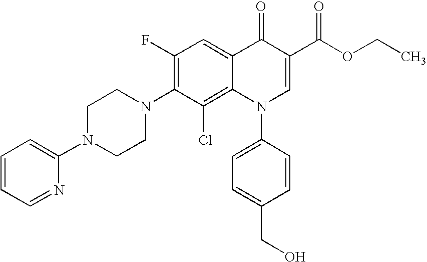

To a solution of ethyl 8-chloro-6,7-difluoro-1-[4-(hydroxymethyl)phenyl]-4-oxo-1,4-dihydroquinoline-3-carboxylate (Intermediate F, 2 g, 5.08 mmol), and 1-(2-pyridyl)piperazine (2.49 g, 15.2 mmol) in 10 mL of dry DMSO was added Hunig's base (6.56 g, 50.8 mmol). The solution was heated at 90° C. overnight. Precipitates formed. The mixture was diluted with ethyl acetate and filtered. The solid washed with ethyl acetate and then hexane to give the desired product as a white powder (2.03 g, 74%). ¹H NMR (DMSO-d₆): δ 8.34 (s, 1H); 8.09 (m, 1H); 7.94 (d, J=12, 1H); 7.53 (m, 5H); 6.83 (d, J=8.4, 1H); 6.64 (m, 1H); 5.38 (t, 1H); 4.59 (d, J=5.6, 2H); 4.22 (q, 2H); 3.58 (s, broad, 4H); 3.23 (s, 4H); 1.26 (t, 3H). MS [M+H]⁺: 537.1 m/z. Calcd 536. RT (LC-MS): 1.97 min.

### Step 2: Preparation of ethyl 1-[4-(bromomethyl)phenyl]-8-chloro-6-fluoro-4-oxo-7-(4-pyridin-2-ylpiperazin-1-yl)-1,4-dihydroquinoline-3-carboxylate

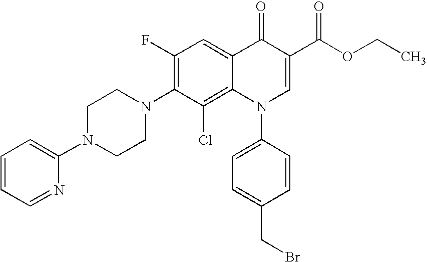

A solution of ethyl 8-chloro-6-fluoro-1-[4-(hydroxymethyl)phenyl]-4-oxo-7-(4-pyridin-2-ylpiperazin-1-yl)-1,4-dihydroquinoline-3-carboxylate (2 g, 3.72 mmol) in CH₂Cl₂ (50 mL) was treated with PBr₃ (2.8 mL, 1M solution in CH₂Cl₂) and then stirred at rt overnight. The reaction mixture was then concentrated. The crude product was purified by silica gel column (CH₂Cl₂ with 1 to 3% of 2M NH₃ in methanol) to give the desired product as a light yellow powder (1.68 g, 71%). ¹H NMR (DMSO-d₆): δ 8.37 (s, 1H); 8.09 (m, 1H); 7.94 (d, J=12, 1H); 7.63 (m, 2H); 7.53 (m, 2H); 6.84 (d, J=8.8, 1H); 6.64 (m, 1H); 4.79 (s, 2H); 4.22 (q, 2H); 3.55 (s, broad, 4H); 3.16 (s, 4H); 1.26 (t, 3H). MS [M+H]⁺: 599.1 m/z. Calcd 599. RT (LC-MS): 2.46 min.

### Step 3: Preparation of ethyl 8-chloro-1-{4-[(cyclopentylamino)-methyl]phenyl}-6-fluoro-4-oxo-7-(4-pyridin-2-ylpiperazin-1-yl)-1,4-dihydro-quinoline-3-carboxylate

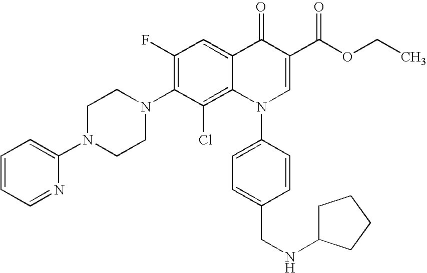

To a solution of ethyl 1-[4-(bromomethyl)phenyl]-8-chloro-6-fluoro-4-oxo-7-(4-pyridin-2-ylpiperazin-1-yl)-1,4-dihydroquinoline-3-carboxylate (3.4 g, 5.67 mmol) in 20 mL of CH₂Cl₂ was added cyclopentylamine (4.8 g, 56.7 mmol) at rt. The solution was stirred at rt overnight. After the reaction was over (followed by TLC), the solvent was removed. The crude product was purified by silica gel column (CH₂Cl₂ with 1% to 5% of 2M NH₃ in methanol) to give the desired product as a white powder (2.31 g, 67%). ¹H NMR (DMSO-d₆): δ 8.33 (s, 1H); 8.09 (m, 1H); 7.94 (d, J=12, 1H); 7.53 (m, 5H); 6.83 (d, J=8.8, 1H); 6.64(m, 1H); 4.22 (q, 2H); 3.75 (s, 2H); 3.32 (s, broad, 4H); 3.23 (s, 4H); 3.04 (t, 1H); 1.74 (m, 2H); 1.65 (m, 2H); 1.48(m, 2H); 1.42 (m, 2H); 1.26 (t, 3H). MS [M+H]⁺: 604.2 m/z. Calcd 603. RT (LC-MS): 1.84 min. TLC (CH₂Cl₂/2M NH₃ in MeOH 95/5) R_{f}=0.32

### Step 4: Preparation of the Title Compound

The solution of ethyl 8-chloro-1-{4-[(cyclopentylamino)methyl]phenyl}-6-fluoro-4-oxo-7-(4-pyridin-2-ylpiperazin-1-yl)-1,4-dihydroquinoline-3-carboxylate (2.3 g, 3.81 mmol) in iPrOH/HCl/H₂O (50 mL/10 mL/5 mL) was heated at 100° C. overnight. The mixture was cooled to rt and ether was added. The precipitate formed was filtered and washed with 2-propanol/hexane to give the desired product as a light yellow powder (2 g, 81%). ¹H NMR (DMSO-d₆): δ 9.46 (s, broad, 2H); 8.54 (s, 1H); 8.14 (d, J=12, 1H); 8.03 (d, J=5.6, 1H); 7.91 (m, 1H); 7.79 (d, J=8, 2H); 7.69 (d J=8, 2H); 7.32 (m, 1H); 6.90 (m, 1H); 4.24 (m, 2H); 3.79 (s, broad, 4H); 3.48 (m, 1H); 3.37 (s, 4H); 2.02 (m, 2H); 1.74 (m, 4H); 1.53 (m, 2H). MS [M+H]⁺: 576.3 m/z. Calcd 575. RT (LC-MS): 2.42 min

### Example 57

### Preparation of 8-chloro-1-{4-[(cyclopentylamino)methyl]-phenyl}-6-fluoro-4-oxo-7-(4-pyrimidin-2-ylpiperazin-1-yl)-1,4-dihydroquinoline-3-carboxylic acid

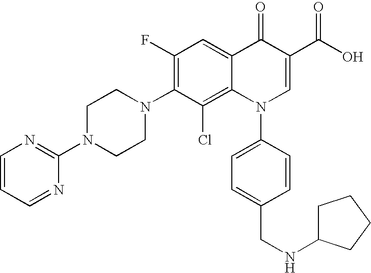

The example was prepared using the procedure as described for the synthesis of Example 56 except 2-piperazin-1-ylpyrimidine was used instead of 1-pyridin-2-ylpiperazine in step 1. LC-MS: 577.2 [M+H]⁺, RT 2.94 min.

### Example 58

### Preparation of 8-chloro-1-{4-[(cyclobutylamino)methyl]-phenyl}-6-fluoro-4-oxo-7-(4-pyridin-2-ylpiperazin-1-yl)-1,4-dihydroquinoline-3-carboxylic acid

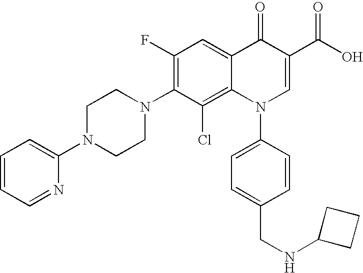

The example was prepared using the procedure as described for the synthesis of Example 56 except cyclobutylamine was used instead of cyclopentylamine in step 3. ¹H NMR (DMSO-d₆): [symbol] 9.64 (s, broad, 2H); 8.54 (s, 1H); 8.14 (d, J=12, 1H); 8.04 (d, J=4.8, 1H); 7.90 (m, 1H); 7.75 (d, J=8.4, 2H); 7.69 (d J=8.4, 2H); 7.29 (m, 1H); 6.89 (m, 1H); 4.14 (m, 2H); 3.94 (m, broad, 5H); 3.37 (s, 4H); 2.29 (m, 4H); 1.83 (m, 2H). LC-MS: 562.1 [M+H]⁺, RT 1.94 nm in.

### Example 59

### Preparation of 8-chloro-1-{4-[(cyclopropylamino)-methyl]phenyl}-6-fluoro-4-oxo-7-(4-pyridin-2-ylpiperazin-1-yl)-1,4-dihydroquinoline-3-carboxylic acid

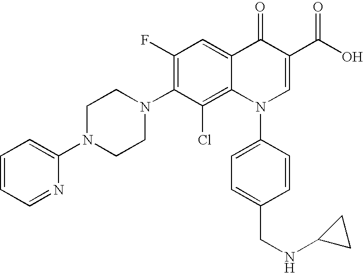

The example was prepared using the procedure as described for the synthesis of Example 56 except aziridine was used instead of cyclopentylamine in step 3. LC-MS: 548 [M+H]⁺, RT 1.81 min.

### Example 60

### Preparation of 8-chloro-1-{4-[(cyclopropylamino)methyl]-phenyl}-6-fluoro-4-oxo-7-(4-pyrimidin-2-ylpiperazin-1-yl)-1,4-dihydroquinoline-3-carboxylic acid

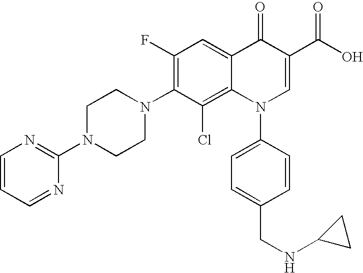

The example was prepared using the procedure as described for the synthesis of Example 56 except aziridine was used instead of cyclopentylamine in step 3 and 2-piperazin-1-ylpyrimidine was used instead of 1-pyridin-2-ylpiperazine in step 1. LC-MS: 549 [M+H]⁺, RT 2.33 min.

### Example 61

### Preparation of 8-chloro-1-(4-{[cyclohexyl(methyl)amino]-methyl}phenyl)-6-fluoro-4-oxo-7-(4-pyridin-2-ylpiperazin-1-yl)-1,4-dihydroquinoline-3-carboxylic acid

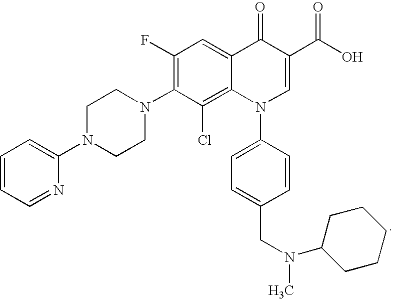

The example was prepared using the procedure as described for the synthesis of Example 56 except N-methylcyclohexylamine was used instead of cyclopentylamine in step 1. LC-MS: 604 [M+H]⁺, RT 2.02 min.

### Example 62

### Preparation of 8-chloro-1-(4-{[cyclohexyl(methyl)amino]-methyl}phenyl)-6-fluoro-4-oxo-7-(4-pyrimidin-2-ylpiperazin-1-yl)-1,4-dihydroquinoline-3-carboxylic acid

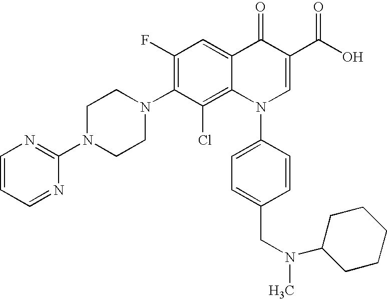

The example was prepared using the procedure as described for the synthesis of Example 56 except N-methylcyclohexylamine was used instead of cyclopentylamine in step 3 and 2-piperazin-1-ylpyrimidine was used instead of 1-pyridin-2-ylpiperazine in step 1. LC-MS: 605.7 [M+H]⁺, RT 2.54 min.

### Example 63

### Preparation of 8-chloro-6-fluoro-7-[4-(2-fluorophenyl)-piperazin-1-yl]-4-oxo-1-[3-(piperidin-1-ylmethyl)phenyl]-1,4-dihydroquinoline-3-carboxylic acid

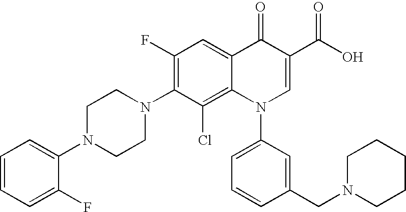

### Step 1: Synthesis of ethyl-2-(3-chloro-2,4,5-trifluorobenzoyl)-3-{[3-(piperidin-1-ylmethyl)phenyl]amino}acrylate

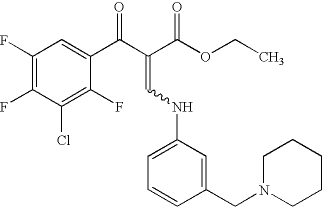

Ethyl-2-(3-chloro-2,4,5-trifluorobenzoyl)-3-ethoxyacrylate (5.0 g, 14.8 mmol) was dissolved in EtOH (abs, 50.0 mL) then cooled to 0° C. [3-(Piperidin-1-ylmethyl)phenyl]amine (step 1 product of Example 137, 2.83 g, 14.8 mmol) was added and the mixture was allowed to warm to room temperature and stir for 2 h. LC-MS analysis showed the reaction was complete. The precipitate was filtered off and the filtrate was concentrated in vacuo to leave a volume of about. 20 mL EtOH. After standing 30 min at room temperature, more precipitate was collected and the combined solids were dried in vacuo to give 5.0 g (70%) of ethyl-2-(3-chloro-2,4,5-trifluorobenzoyl)-3-{[3-(piperidin-1-ylmethyl)phenyl]-amino}acrylate which was carried to next step without further purification.

### Step 2: Synthesis of ethyl 8-chloro-6,7-difluoro-4-oxo-1-[3-(piperidin-1-ylmethyl)phenyl]-1,4-dihydroquinoline-3-carboxylate

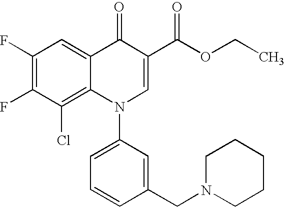

Ethyl-2-(3-chloro-2,4,5-trifluorobenzoyl)-3-{[3-(piperidin-1-ylmethyl)phenyl]amino}acrylate (5.0 g, 10.4 mmol) was dissolved in THF (10 mL) and K₂CO₃ (4.31 g, 31.2 mmol, 3 equiv.) and 18-crown-6 (1.92 g, 3.12 mmol, 0.3 equiv.) were added. The mixture was heated to reflux for 18 h; LC-MS showed the starting material was gone. The reaction was cooled to room temperature, filtered and the solids were rinsed with THF. The solvent was removed from the filtrate in vacuo and hexanes were added to the oil and the mixture was stirred 18 h. The precipitate was filtered and dried in a drying oven to give 4.50 g (94%) of ethyl 8-chloro-6,7-difluoro-4-oxo-1-[3-(piperidin-1-ylmethyl)phenyl]-1,4-dihydro-quinoline-3-carboxylate as an off-white solid.

### Step 3: Synthesis of ethyl 8-chloro-6,7-difluoro-4-oxo-1-[3-(piperidin-1-ylmethyl)phenyl]-1,4-dihydroquinoline-3-carboxylate

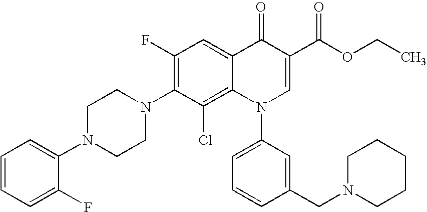

1-(2-Fluorophenyl)piperazine was added to the ethyl 8-chloro-6,7-difluoro-4-oxo-1-[3-(piperidin-1-ylmethyl)phenyl]-1,4-dihydroquinoline-3-carboxylate following the procedure given in Step 1 of Example 119 to give ethyl 8-chloro-6-fluoro-7-[4-(2-fluorophenyl)piperazin-1-yl]-4-oxo-1-[3-(piperidin-1-yl-methyl)-phenyl]-1,4-dihydroquinoline-3-carboxylate. ¹H NMR (acetone-d₆) δ 8.46 (s, 1H), 8.00 (d, 1H), 7.51-7.56 (m, 2H), 7.46-7.50 (m, 2H), 7.04-7.12 (m, 3H), 6.96-7.01 (m, 1H), 4.26 (dd, 2H), 3.53 (dd, 2H), 3.42 (br s, 4H), 3.18 (br s, 4H), 2.41 (br s, 4H), 1.52-1.58 (m, 4H), 1.43-1.45 (m, 2H), 1.31 (t, 3H); LC-MS RT 2.65 min; [M+H]⁺ 621.8.

### Step 4: Synthesis of the Title Compound

The ester was hydrolyzed following the procedure given in Step 2 of Example 119. The residue was purified using HPLC to provide 8-chloro-6-fluoro-7-[4-(2-fluorophenyl)piperazin-1-yl]-4-oxo-1-[3-(piperidin-1-ylmethyl)phenyl]-1,4-dihydroquinoline-3-carboxylic acid trifluoroacetate. ¹H NMR (CD₃OD) δ 8.73 (s, 1H), 8.09 (d, 1H), 7.67-7.23 (m, 4H), 6.97-7.08 (m, 4H), 4.42 (dd, 2H), 3.46-3.57 (m, 6H), 3.17 (br s, 4H), 2.98-3.04 (m, 2H), 1.93-1.99 (m, 2H), 1.77-1.88 (m, 3H), 1.51-1.55 (m, 1H); LC-MS RT 2.86 min; [M+H]⁺ 593.3.

### Example 64

### Preparation of 8-chloro-7-[4-(2,4-difluorophenyl)piperazin-1-yl]-6-fluoro-4-oxo-1-[3-(piperidin-1-ylmethyl)phenyl]-1,4-dihydroquinoline-3-carboxylic acid

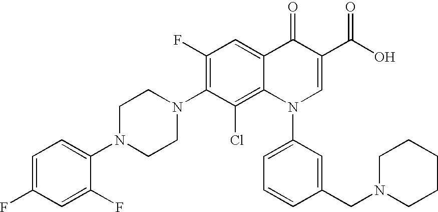

The example was prepared using the procedure as described for the synthesis of Example 63 except 1-(2,4-difluorophenyl)piperazine was used instead of 1-(2-fluorophenyl)piperazine in step 3. LC-MS: 611.3 [M+H]⁺, RT 2.91 nm in.

### Example 65

### Preparation of 8-chloro-6-fluoro-4-oxo-1-[3-(piperidin-1-yl-methyl)phenyl]-7-(4-pyridin-2-ylpiperazin-1-yl)-1,4-dihydroquinoline-3-carboxylic acid

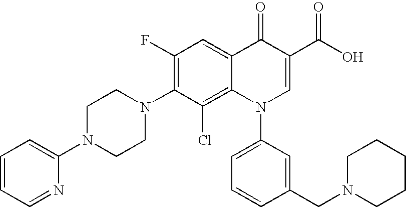

The example was prepared using the procedure as described for the synthesis of Example 63 except 1-pyridin-2-ylpiperazine was used instead of 1-(2-fluorophenyl)piperazine in step 3. LC-MS: 576.6 [M+H]⁺, RT 1.84 nm in.

### Example 66

### Preparation of 8-chloro-6-fluoro-4-oxo-1-[3-(piperidin-1-yl-methyl)phenyl]-7-(4-pyrimidin-2-ylpiperazin-1-yl)-1,4-dihydroquinoline-3-carboxylic acid

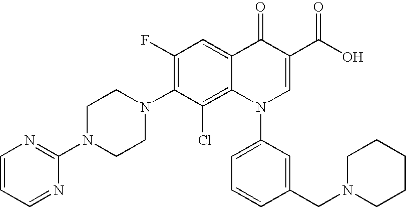

The example was prepared using the procedure as described for the synthesis of Example 63 except 2-piperazin-1-ylpyrimidine was used instead of 1-(2-fluorophenyl)piperazine in step 3. LC-MS: 578.1 [M+H]⁺, RT 2.34 min.

### Example 67

### Preparation of 8-chloro-6-fluoro-7-[4-(4-fluorophenyl)-piperazin-1-yl]-4-oxo-1-[3-(piperidin-1-ylmethyl)phenyl]-1,4-dihydroquinoline-3-carboxylic acid

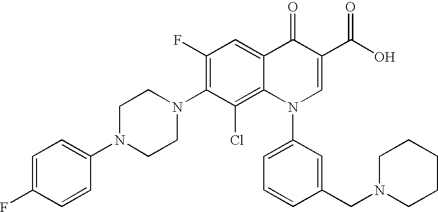

The example was prepared using the procedure as described for the synthesis of Example 63 except 1-(4-fluorophenyl)piperazine was used instead of 1-(2-fluorophenyl)piperazine in step 3. LC-MS: 594 [M+H]⁺, RT 2.62 min.

### Example 68

### Preparation of 8-chloro-7-[4-(3-chlorophenyl)piperazin-1-yl]-6-fluoro-4-oxo-1-[3-(piperidin-1-ylmethyl)phenyl]-1,4-dihydroquinoline-3-carboxylic acid

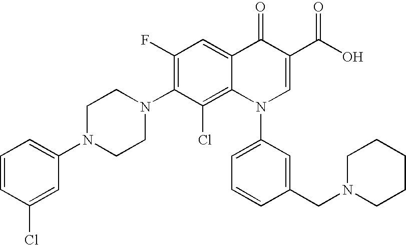

The example was prepared using the procedure as described for the synthesis of Example 63 except 1-(3-chlorophenyl)piperazine was used instead of 1-(2-fluorophenyl)piperazine in step 3. LC-MS: 609.9 [M+H]⁺, RT 2.85 min.

### Example 69

### 8-chloro-1-{4-[2-(dimethylamino)ethyl]phenyl}-6-fluoro-4-oxo-7-(4-pyridin-2-ylpiperazin-1-yl)-1,4-dihydroquinoline-3-carboxylic acid

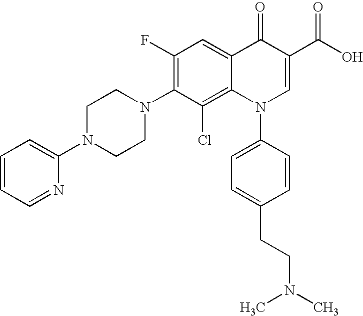

### Step 1: Preparation of N,N-dimethyl-2-(4-nitrophenyl)ethanamine

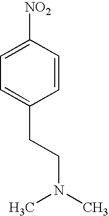

A solution of 4-nitrophenethylbromide (3.1 g) in THF (20 mL) was added 20 mL of dimethylamine (2N in methanol). The solution was stirred at room temperature overnight. The mixture was heated further at 80° C. over night, then the solvent was concentrated. The residue was purified with silica gel column chromatography to give 1.0 g of the desired product in 38% yield.

### Step 2: Preparation of 4-[2-(dimethylamino)ethyl]aniline

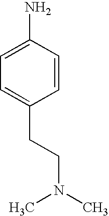

A solution of 5-nitro-2-(pyrrolidin-1-ylmethyl)pyridine (1 g) was purged with Ar for 5 min, then the 10% Pd/C catalyst was added, followed by EtOAc (100 mL). The mixture was stirred under hydrogen atmosphere (baloon) for 8 hrs. The reaction mixture was passed through a Celite® bed and the filtrate was concentrated to give 0.5 g of the desired product in 59% yield.

### Step 3: Preparation of 8-chloro-1-{4-[2-(dimethylamino)ethyl]phenyl}-6,7-difluoro-4-oxo-1,4-dihydroquinoline-3-carboxylic acid

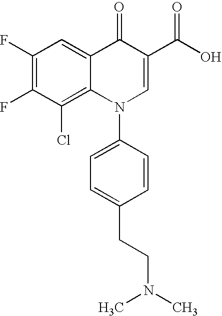

A solution of ethyl 2-(3-chloro-2,4,5-trifluorobenzoyl)-3-ethoxyacrylate (1.0 g) in EtOH (40 mL) was treated with 4-[2-(dimethylamino)ethyl]aniline (0.5 g) at −10° C. The reaction was stirred at room temperature over night. After the solvent was concentrated, the residue was dissolved in 50 mL of CH₃CN. To this solution was added 0.24 g of 18-crown-6, 0.82 g of potassium carbonate and the reaction mixture was heated to reflux for 4 h. The resulting precipitate was filtered off and solvent was concentrated to give white precipitate. The ester was hydrolyzed under acidic condition using HCl (aq, conc), water and ethanol under reflux conditions over night to give 0.12 g of white precipitate (8% yield). ¹H NMR (CD₃OD) δ 8.7 (s, 1H), 8.4 (t, 1H), 7.6 (s, 4H), 3.5 (q, 2H), 3.2 (q, 2H), 3.0 (s, 6H). LC-MS: 407 [M+H]⁺, RT 2.20 min.

### Step 4: Preparation of the Title Compound

To a solution of 8-chloro-1-{4-[2-(dimethylamino)ethyl]phenyl}-6,7-difluoro-4-oxo-1,4-dihydroquinoline-3-carboxylic acid (30 mg) was added 1-(2-pyridyl)piperazine (60 mg), and DABCO (40 mg) in CH₃CN (2 mL) and the reaction mixture was heated at 90° C. overnight. The reaction mixture was cooled to room temperature which resulted in formation of the desired product as a white precipitate. The crude product was purified using HPLC to give 3.6 mg of 8-chloro-1-{4-[2-(dimethylamino)ethyl]phenyl}-6-fluoro-4-oxo-7-(4-pyridin-2-ylpiperazin-1-yl)-1,4-dihydroquinoline-3-carboxylic acid (9% yield). ¹H NMR (CD₃OD) δ 8.7 (s, 1H), 8.2 (d, 1H), 8.05 (t, 1H), 7.96 (d, 1H), 7.5 (s, 4H), 7.4 (d, 1H), 7.0 (t, 1H), 3.82 (s, 4H), 3.5 (m, 6H), 3.2 (q, 2H), 3.0 (s, 6H). LC-MS 550 (mw+1), RT 1.77 min.

### Example 70

### Preparation of 8-chloro-7-[4-(4-chlorophenyl)piperazin-1-yl]-1-{4-[2-(dimethylamino)ethyl]phenyl}-6-fluoro-4-oxo-1,4-dihydroquinoline-3-carboxylic acid

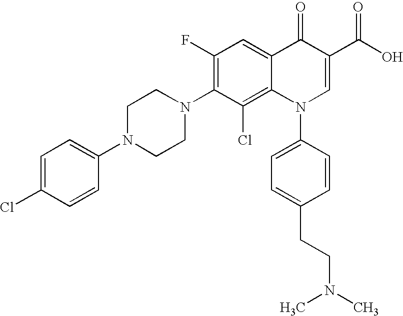

The example was prepared using the procedure as described for the synthesis of Example 69 except 1-(3-chlorophenyl)piperazine was used instead of 1-pyridin-2-ylpiperazine in the final step. ¹H NMR (CD₃OD): δ 8.5 (s, 1H), 8.16 (d, 1H), 7.55 (m, 4H), 7.2 (d, 2H), 6.95 (d, 2H), 3.44 (m, 6H), 3.2 (m, 4H), 3.0 (s, 6H). LC-MS: 609.9 [M+H]⁺, RT 2.85 min.

### Example 71

### Preparation of 8-chloro-1-{4-[2-(dimethylamino)ethyl]phenyl}-6-fluoro-4-oxo-7-(4-pyrimidin-2-ylpiperazin-1-yl)-1,4-dihydroquinoline-3-carboxylic acid

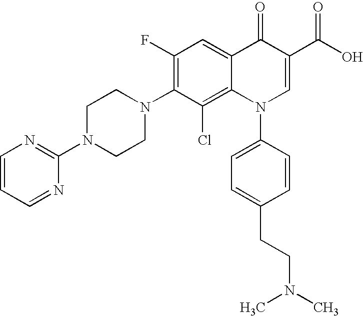

The example was prepared using the procedure as described for the synthesis of Example 69 except 2-piperazin-1-ylpyrimidine was used instead of 1-pyridin-2-ylpiperazine in the final step. ¹H NMR (CD₃OD): δ 8.7 (s, 1H), 8.3 (d, 1H), 8.16 (t, 1H), 7.5 (m, 4H), 6.6 (t, 1H), 3.8 (s, 4H), 3.5 (q, 2H), 3.36 (s, 4H), 3.2 (q, 2H), 3.0 (s, 6H). LC-MS: 609.9 [M+H]⁺, RT 2.85 min.

### Example 72

### Preparation of 6-fluoro-8-methoxy-4-oxo-7-(4-pyridin-2-ylpiperazin-1-yl)-1-[4-(pyrrolidin-1-ylmethyl)phenyl]-1,4-dihydroquinoline-3-carboxylic acid

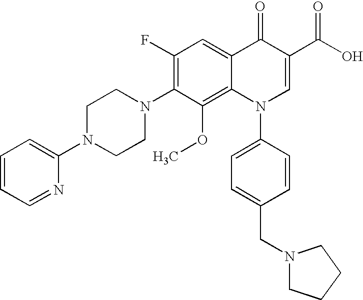

### Step 1. Synthesis of 6,7-difluoro-8-methoxy-4-oxo-1-[4-(pyrrolidin-1-ylmethyl)phenyl]-1,4-dihydroquinoline-3-carboxylic acid

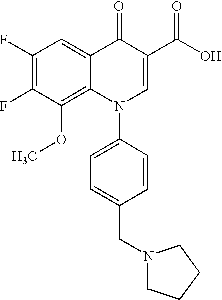

Ethyl 6,7-difluoro-8-methoxy-4-oxo-1-[4-(pyrrolidin-1-ylmethyl)phenyl]-1,4-dihydroquinoline-3-carboxylate (Intermediate C, 3.06 g, 6.9 mmol) was added to a mixture of EtOH/HCl/H₂O (100/12.5/12.5, 100 mL) and then heated to 70° C. for 18 h. The solution was cooled to room temperature and the precipitate was collected by filtration, rinsed with water, rinsed with copious amounts of ether, and then dried to give 2.65 g (93%) of 6,7-difluoro-8-methoxy-4-oxo-1-[4-(pyrrolidin-1-ylmethyl)phenyl]-1,4-dihydroquinoline-3-carboxylic acid as a colorless solid. ¹H NMR δ 10.9 (s, 1H), 8.5 (s, 1H), 8.1 (t, 1H), 7.8-7.7 (dd, 4H), 4.5 (d, 2H), 3.4 (m, 4H), 3.1 (s, 3H), 2.0-1.9 (m, 4H).

### Step 2. Synthesis of {6,7-difluoro-8-methoxy-4-oxo-1-[4-(pyrrolidin-1-ylmethyl)phenyl]-1,4-dihydroquinolin-3-yl}carbonyl difluoridoborate

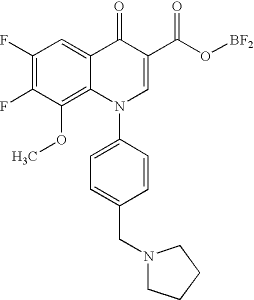

Boron trifluoride etherate (13 mL, 106 mmol) was added slowly to a solution of 6,7-difluoro-8-methoxy-4-oxo-1-[4-(pyrrolidin-1-ylmethyl)phenyl]-1,4-dihydroquinoline-3-carboxylic acid (step 1 product, 2.65 g, 6.4 mmol) in THF (20 mL) at room temperature. The reaction was heated to 70° C. for 18 h then cooled to rt, diluted with Et₂O (20 mL) and the precipitate was collected by filtration to give 3.3 g (slightly wet) of {6,7-difluoro-8-methoxy-4-oxo-1-[4-(pyrrolidin-1-ylmethyl)phenyl]-1,4-dihydroquinolin-3-yl}carbonyl difluoridoborate as a colorless solid. ¹H NMR δ 9.8 (s, 1H), 9.1 (s, 1H), 8.5-8.4 (t, 1H), 7.8-7.7 (dd, 4H), 4.5 (d, 2H), 3.4 (m, 4H), 3.2 (m, 3H), 2.0 (m, 4H).

### Step 3. Synthesis of the Title Compound

{6,7-difluoro-8-methoxy-4-oxo-1-[4-(pyrrolidin-1-ylmethyl)phenyl]-1,4-dihydroquinolin-3-yl}carbonyl difluoridoborate (step 2 product, 3.30 g, approx 6.4 mmol) was dissolved in acetonitrile (50.0 mL) and 1-pyridin-2-ylpiperazine (4.66 g, 28.5 mmol) was added. The solution was heated to 40° C. for 18 h then cooled to room temperature. The acetonitrile was removed in vacuo and the solids were dissolved in water and neutralized to pH 7 with 1 M HCl. The precipitate was stirred for 18 h longer in water until it solidified. The precipitate was then collected by filtration to give 3.76 g colorless solid. The solid was dissolved in EtOH (40.0 mL), H₂O (10.0 mL), and triethylamine (5.2 mL) and the solution was heated to reflux for 18 h. The reaction was cooled to room temperature and the solvent was removed in vacuo. The solid was then taken up in water and basified to pH 12 with 50% NaOH (aq) then neutralized back to, pH 7 with conc. HCl. The water layer was extracted with chloroform/isopropanol (3:1) 4 times. The combined chloroform/isopropanol layers were extracted with several water washes until water remains colorless. Solvent was removed in vacuo from the organic layers to give 6-fluoro-8-methoxy-4-oxo-7-(4-pyridin-2-ylpiperazin-1-yl)-1-[4-(pyrrolidin-1-ylmethyl)phenyl]-1,4-dihydroquinoline-3-carboxylic acid. ¹H NMR: δ 10.7 (s, 1H), 8.4 (s, 1H), 8.0 (d, 1H), 7.9 (dd, 1H) 7.7 (m, 4H), 7.3 (bs, 1H), 6.8 (bs, 1H), 4.5 (d, 2H), 3.7 (m, 4H), 3.6 (m, 8H), 3.2 (m, 3H), 2.0-1.8 (m, 4H). LC-MS: 607 [M+H]⁺, RT 2.49 min.

### Example 73

### Preparation of 6-fluoro-8-methoxy-4-oxo-7-(4-pyrimidin-2-ylpiperazin-1-yl)-1-[4-(pyrrolidin-1-ylmethyl)phenyl]-1,4-dihydroquinoline-3-carboxylic acid

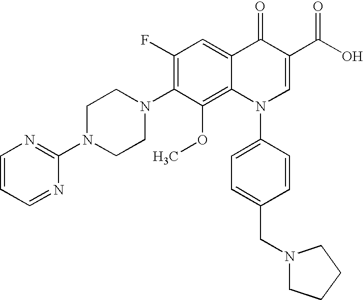

Example 73 was prepared using the procedure as described for the synthesis of Example 72 except 2-piperazin-1-ylpyrimidine was used instead of 1-pyridin-2-ylpiperazine in step 3. LC-MS: 559 [M+H]⁺, RT 2.80 min.

### Example 74

### Preparation of 6-fluoro-7-[4-(4-fluorophenyl)piperazin-1-yl]-8-methoxy-4-oxo-1-[4-(pyrrolidin-1-ylmethyl)phenyl]-1,4-dihydroquinoline-3-carboxylic acid

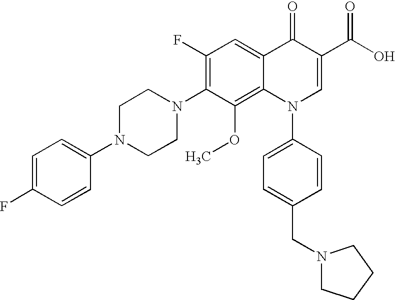

The example was prepared using the procedure as described for the synthesis of Example 72 except 1-(4-fluorophenyl)piperazine was used instead of 1-pyridin-2-ylpiperazine in step 3. LC-MS: 575.3 [M+H]⁺, RT 2.65 min.

### Example 75

### Preparation of 6-fluoro-7-[4-(2-fluorophenyl)piperazin-1-yl]-8-methoxy-4-oxo-1-[4-(pyrrolidin-1-ylmethyl)phenyl]-1,4-dihydroquinoline-3-carboxylic acid

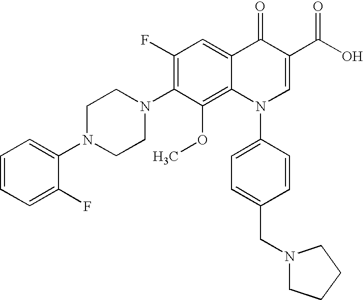

The example was prepared using the procedure as described for the synthesis of Example 72 except 1-(2-fluorophenyl)piperazine was used instead of 1-pyridin-2-ylpiperazine in step 3. LC-MS: 575.8 [M+H]⁺, RT 2.71 min.

### Example 76

### Preparation of 7-[4-(2,4-difluorophenyl)piperazin-1-yl]-6-fluoro-8-methoxy-4-oxo-1-[4-(pyrrolidin-1-ylmethyl)phenyl]-1,4-dihydroquinoline-3-carboxylic acid

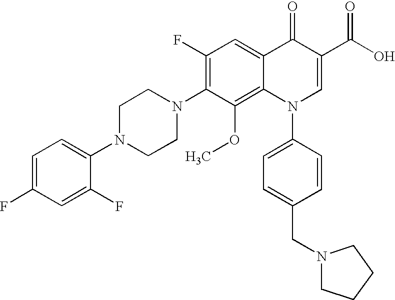

The example was prepared using the procedure as described for the synthesis of Example 72 except 1-(2,4-difluorophenyl)piperazine was used instead of 1-pyridin-2-ylpiperazine in step 3. LC-MS: 594 [M+H]⁺, RT 2.76 min.

### Example 77

### Preparation of 7-[4-(4-cyanophenyl)piperazin-1-yl]-6-fluoro-8-methoxy-4-oxo-1-[4-(pyrrolidin-1-ylmethyl)phenyl]-1,4-dihydroquinoline-3-carboxylic acid

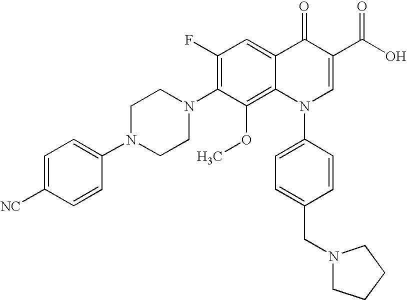

The example was prepared using the procedure as described for the synthesis of Example 72 except 4-piperazin-1-ylbenzonitrile was used instead of 1-pyridin-2-ylpiperazine in step 3. LC-MS: 582.2 [M+H]⁺, RT 2.65 nm n.

### Example 78

### Preparation of 7-[4-(4-acetylphenyl)piperazin-1-yl]-6-fluoro-8-methoxy-4-oxo-1-[4-(pyrrolidin-1-ylmethyl)phenyl]-1,4-dihydroquinoline-3-carboxylic acid

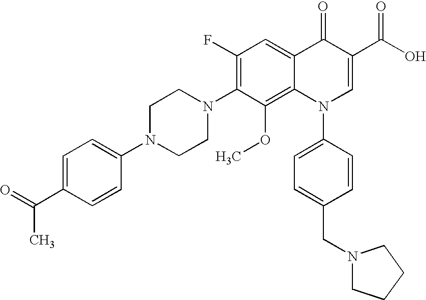

The example was prepared using the procedure as described for the synthesis of Example 72 except 1-(4-piperazin-1-ylphenyl)ethanone was used instead of 1-pyridin-2-ylpiperazine in step 3. LC-MS: 587.3 [M+H]⁺, RT 2.33 min.

### Example 79

### Preparation of 7-[4-(3-cyanopyridin-2-yl)piperazin-1-yl]-6-fluoro-8-methoxy-4-oxo-1-[4-(pyrrolidin-1-ylmethyl)phenyl]-1,4-dihydroquinoline-3-carboxylic acid

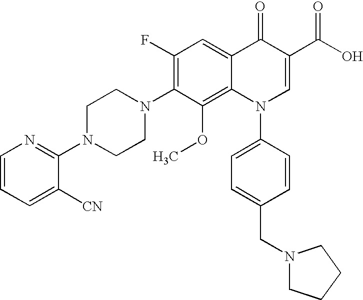

The example was prepared using the procedure as described for the synthesis of Example 72 except 2-piperazin-1-ylnicotinonitrile was used instead of 1-pyridin-2-ylpiperazine in step 3. LC-MS: 583.2 [M+H]⁺, RT 2.57 min.

### Example 80

### Preparation of 7-[4-(4-chlorophenyl)piperazin-1-yl]-6-fluoro-8-methoxy-4-oxo-1-[4-(pyrrolidin-1-ylmethyl)phenyl]-1,4-dihydroquinoline-3-carboxylic acid

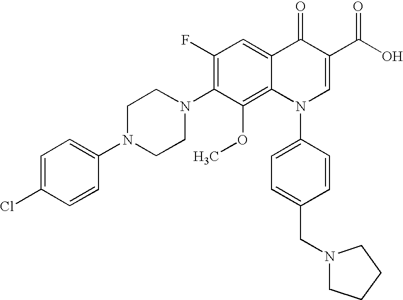

The example was prepared using the procedure as described for the synthesis of Example 72 except 1-(4-chlorophenyl)piperazine was used instead of 1-pyridin-2-ylpiperazine in step 3. LC-MS: 591.2 [M+H]⁺, RT 2.90 min.

### Example 81

### Preparation of 7-[4-(2-cyanophenyl)piperazin-1-yl]-6-fluoro-8-methoxy-4-oxo-1-[4-(pyrrolidin-1-ylmethyl)phenyl]-1,4-dihydroquinoline-3-carboxylic acid

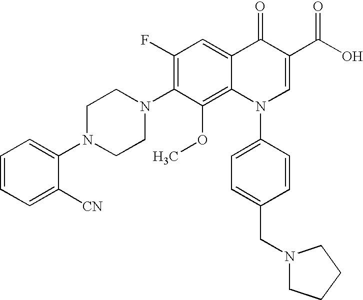

The example was prepared using the procedure as described for the synthesis of Example 72 except 2-piperazin-1-ylbenzonitrile was used instead of 1-pyridin-2-ylpiperazine in step 3. LC-MS: 582.2 [M+H]⁺, RT 2.71 min.

### Example 82

### Preparation of 6-fluoro-8-methoxy-4-oxo-1-[4-(pyrrolidin-1-ylmethyl)phenyl]-7-{4-[4-(trifluoromethyl)phenylpiperazin-1-yl}-1,4-dihydroquinoline-3-carboxylic acid

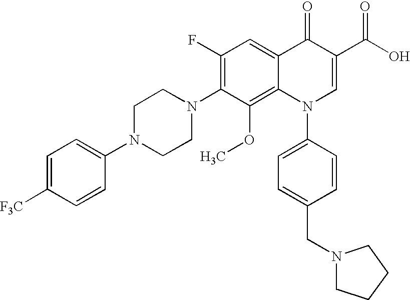

The example was prepared using the procedure as described for the synthesis of Example 72 except 1-[4-(trifluoromethyl)phenyl]piperazine was used instead of 1-pyridin-2-ylpiperazine in step 3. LC-MS: 625.2 [M+H]⁺, RT 3.00 min.

### Example 83

### Preparation of 6-fluoro-8-methoxy-7-[4-(2-methoxyphenyl)-piperazin-1-yl]-4-oxo-1-[4-(pyrrolidin-1-ylmethyl)phenyl]-1,4-dihydroquinoline-3-carboxylic acid

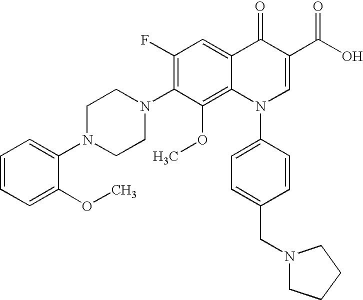

The example was prepared using the procedure as described for the synthesis of Example 72 except 1-[2-(methoxy)phenyl]piperazine was used instead of 1-pyridin-2-ylpiperazine in step 3. LC-MS: 587.2 [M+H]⁺, RT 2.40 min.

### Example 84

### Preparation of 6-fluoro-8-methoxy-7-[4-(2-nitrophenyl)-piperazin-1-yl]-4-oxo-1-[4-(pyrrolidin-1-ylmethyl)phenyl]-1,4-dihydroquinoline-3-carboxylic acid

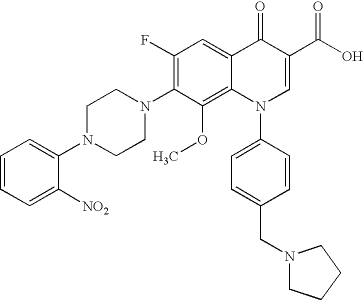

The example was prepared using the procedure as described for the synthesis of Example 72 except 4-(2-nitrophenyl)piperazine was used instead of 1-pyridin-2-ylpiperazine in step 3. LC-MS: 602.2 [M+H]⁺, RT 2.78 min.

### Example 85

### Preparation of 6-fluoro-7-[4-(3-fluorophenyl)piperazin-1-yl]-8-methoxy-4-oxo-1-[4-(pyrrolidin-1-ylmethyl)phenyl]-1,4-dihydroquinoline-3-carboxylic acid

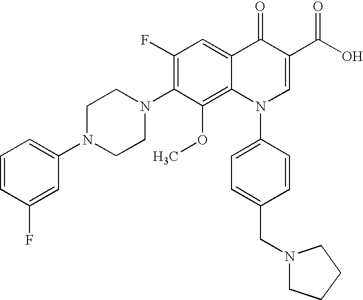

The example was prepared using the procedure as described for the synthesis of Example 72 except 4-(3-fluorophenyl)piperazine was used instead of 1-pyridin-2-ylpiperazine in step 3. LC-MS: 575.2 [M+H]⁺, RT 3.19 min.

### Example 86

### Preparation of 7-[4-(3,4-dichlorophenyl)piperazin-1-yl]-6-fluoro-8-methoxy-4-oxo-1-[4-(pyrrolidin-1-ylmethyl)phenyl]-1,4-dihydroquinoline-3-carboxylic acid

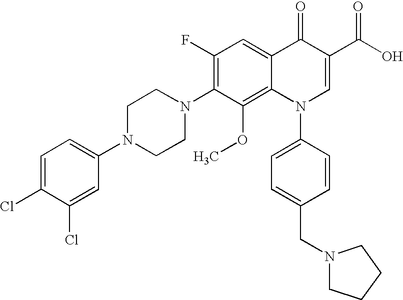

The example was prepared using the procedure as described for the synthesis of Example 72 except 4-(3,4-dichlorophenyl)piperazine was used instead of 1-pyridin-2-ylpiperazine in step 3. LC-MS: 625.9 [M+H]⁺, RT 2.92 min.

### Example 87

### Preparation of 6-fluoro-7-[4-(2-fluoro-4-nitrophenyl)piperazin-1-yl]-8-methoxy-4-oxo-1-[4-(pyrrolidin-1-ylmethyl)phenyl]-1,4-dihydroquinoline-3-carboxylic acid

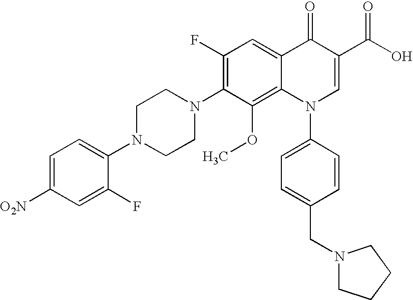

The example was prepared using the procedure as described for the synthesis of Example 72 except 4-(2-fluoro,4-nitrophenyl)piperazine was used instead of 1-pyridin-2-ylpiperazine in step 3. LC-MS: 620.5 [M+H]⁺, RT 2.72 min.

### Example 88

### Preparation of 6-fluoro-8-methoxy-7-{4-[2-(methylthio)phenyl]-piperazin-1-yl}-4-oxo-1-[4-(pyrrolidin-1-ylmethyl)phenyl]-1,4-dihydroquinoline-3-carboxylic acid

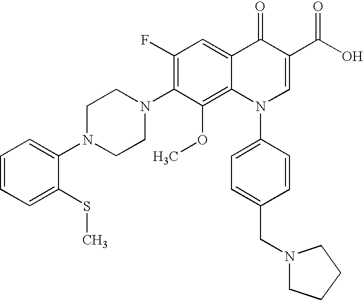

The example was prepared using the procedure as described for the synthesis of Example 72 except 1-[2-(methylthio)phenyl]piperazine was used instead of 1-pyridin-2-ylpiperazine in step 3. LC-MS: 603.2 [M+H]⁺, RT 2.99 min.

### Example 89

### Preparation of 7-[4-(2-cyanophenyl)piperazin-1-yl]-6-fluoro-8-methoxy-4-oxo-1-[4-(piperidin-1-ylmethyl)phenyl]-1,4-dihydroquinoline-3-carboxylic acid

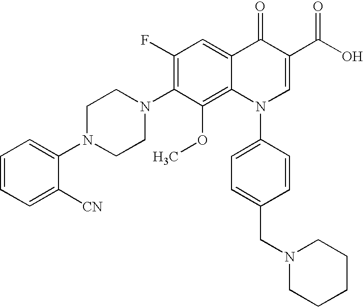

### Step 1: Preparation of ethyl 6,7-difluoro-8-methoxy-4-oxo-1-[4-(piperidin-1-ylmethyl)phenyl]-1,4-dihydroquinoline-3-carboxylate

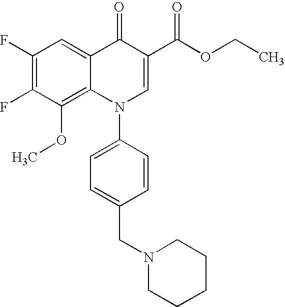

This intermediate was prepared using the procedure as described for the synthesis of Intermediate C except 4-(piperidin-1-ylmethyl)aniline was used instead of 4-pyrrolidin-1-ylmethyl-phenylamine.

### Step 2: Preparation of the Title Compound

The title compound was prepared using procedure as described for the synthesis of Example 72 except ethyl 6,7-difluoro-8-methoxy-4-oxo-1-[4-(piperidin-1-ylmethyl)phenyl]-1,4-dihydroquinoline-3-carboxylate was used instead of ethyl 6,7-difluoro-8-methoxy-4-oxo-1-[4-(pyrrolidin-1-ylmethyl)-phenyl]-1,4-dihydroquinoline-3-carboxylate (Intermediate C) and 2-piperazin-1-ylbenzonitrile was used instead of 1-pyridin-2-ylpiperazine in step 3. LC-MS: 596.5 [M+H]⁺, RT 2.61 min.

### Example 90

### Preparation of 7-[4-(4-chlorophenyl)piperazin-1-yl]-6-fluoro-8-methoxy-4-oxo-1-[4-(piperidin-1-ylmethyl)phenyl]-1,4-dihydroquinoline-3-carboxylic acid

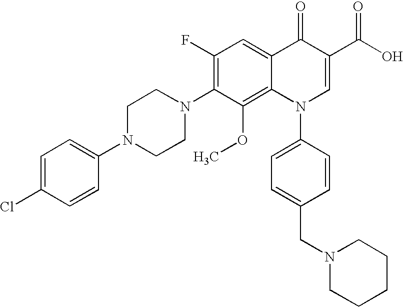

The example was prepared by using procedure as described for the synthesis of Example 89 except 1-(4-chlorophenyl)piperazine was used instead of 2-piperazin-1-ylbenzonitrile in step 2. LC-MS: 596.5 [M+H]⁺, RT 2.61 min. LC-MS: 605.6 [M+H]⁺, RT 2.81 min.

### Example 91

### Preparation of 6-fluoro-8-methoxy-7-[4-(2-nitrophenyl)-piperazin-1-yl]-4-oxo-1-[4-(piperidin-1-ylmethyl)phenyl]-1,4-dihydroquinoline-3-carboxylic acid

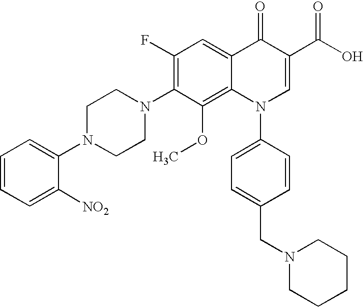

The example was prepared by using procedure as described for the synthesis of Example 89 except 1-(2-nitrophenyl)piperazine was used instead of 2-piperazin-1-ylbenzonitrile in step 2. LC-MS: 616.4 [M+H]⁺, RT 2.70 min.

### Example 92

### Preparation of 1-{4-[(dimethylamino)methyl]phenyl}-6-fluoro-8-methoxy-4-oxo-7-(4-pyridin-2-ylpiperazin-1-yl)-1,4-dihydroquinoline-3-carboxylic acid

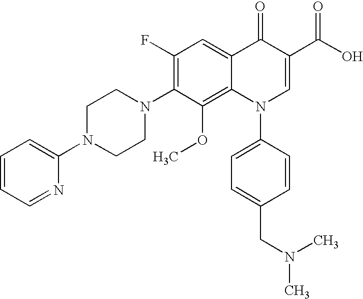

### Step 1: Preparation of ethyl 1-{4-[(dimethylamino)methyl]phenyl}-6,7-difluoro-8-methoxy-4-oxo-1,4-dihydroquinoline-3-carboxylate

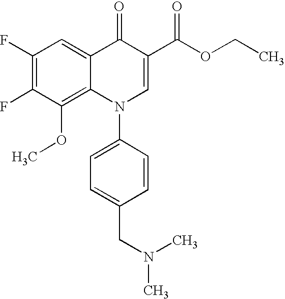

This intermediate was prepared using the procedure as described for the synthesis of Intermediate C except 4-[(dimethylamino)methyl]aniline was used instead of 4-pyrrolidin-1-ylmethyl-phenylamine.

### Step 2: Preparation of the Title Compound

The example was then prepared by using procedure as described for the synthesis of Example 72 except ethyl 1-{4-[(dimethylamino)methyl]phenyl}-6,7-difluoro-8-methoxy-4-oxo-1,4-dihydroquinoline-3-carboxylate was used instead of Ethyl 6,7-difluoro-8-methoxy-4-oxo-1-[4-(pyrrolidin-1-ylmethyl)phenyl]-1,4-dihydroquinoline-3-carboxylate (Intermediate C). ¹H NMR: δ 8.4 (s, 1H), 8.1 (d, 1H), 7.9 (d, 2H), 7.7 (s, 4H), 7.2 (bs, 1H), 6.9 (bs, 1H), 4.4 (d, 2H), 3.2 (t, 3H), 2.7 (m, 4H), 2.4 (m, 4H), 1.1 (t, 6H). LC-MS: 532 [M+H]⁺, RT 1.62 min.

### Example 93

### Preparation of 1-{4-[(dimethylamino)methyl]phenyl}-6-fluoro-7-[4-(4-fluorophenyl)piperazin-1-yl]-8-methoxy-4-oxo-1,4-dihydroquinoline-3-carboxylic acid

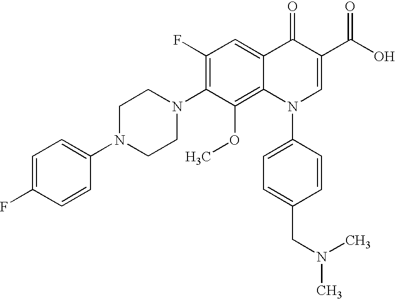

The example was prepared by using the procedure as described for the synthesis of Example 92 except 1-(4-fluorophenyl)piperazine was used instead of 1-pyridin-2-ylpiperazine in step 3. ¹H NMR: δ 8.6 (s, 1H), 8.0 (d, 1H), 7.7 (s, 4H), 7.5 (m, 2H), 7.2 (t, 2H), 4.4 (s, 2H), 3.6 (m, 4H), 3.5 (m, 4H), 3.3 (s, 3H), 2.9 (t, 6H). LC-MS: 549.3 [M+H]⁺, RT 2.52 nm.

### Example 94

### Preparation of 1-{4-[(dimethylamino)methyl]phenyl}-6-fluoro-7-[4-(2-fluorophenyl)piperazin-1-yl]-8-methoxy-4-oxo-1,4-dihydroquinoline-3-carboxylic acid

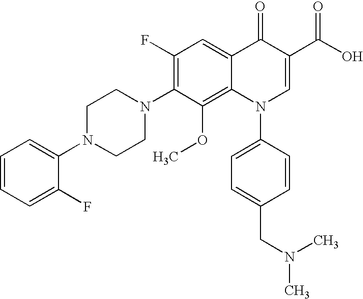

The example was prepared by using the procedure as described for the synthesis of Example 92 except 1-(2-fluorophenyl)piperazine was used instead of 1-pyridin-2-ylpiperazine in step 3. LC-MS: 549.3 [M+H]⁺, RT 2.63 min.

### Example 95

### Preparation of 7-[4-(2-cyanophenyl)piperazin-1-yl]-1-{4-[(dimethylamino)methyl]phenyl}-6-fluoro-8-methoxy-4-oxo-1,4-dihydroquinoline-3-carboxylic acid

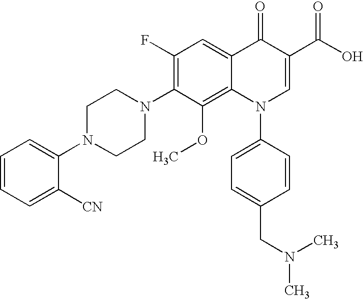

The example was prepared by using the procedure as described for the synthesis of Example 92 except 2-piperazin-1-ylbenzonitrile was used instead of 1-pyridin-2-ylpiperazine in step 3. LC-MS: 556.2 [M+H]⁺, RT 2.69 min.

### Example 96

### Preparation of 1-{4-[(dimethylamino)methyl]phenyl}-6-fluoro-8-methoxy-4-oxo-7-(4-pyrimidin-2-ylpiperazin-1-yl)-1,4-dihydroquinoline-3-carboxylic acid

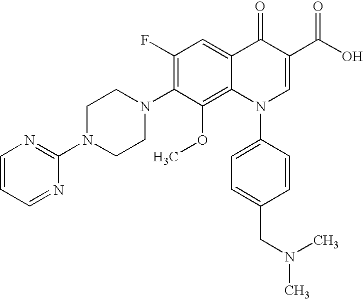

The example was prepared by using the procedure as described for the synthesis of Example 92 except 2-piperazin-1-ylpyrimidine was used instead of 1-pyridin-2-ylpiperazine in step 3. LC-MS: 533.2[M+H]⁺, RT 2.36 min.

### Example 97

### Preparation of 1-{4-[(dimethylamino)methyl]phenyl}-6-fluoro-8-methoxy-4-oxo-7-(4-phenylpiperazin-1-yl)-1,4-dihydroquinoline-3-carboxylic acid

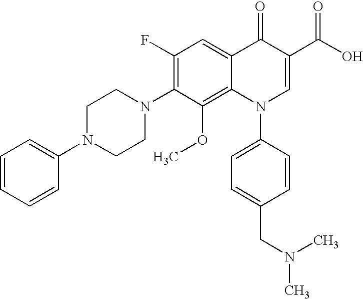

The example was prepared by using the procedure as described for the synthesis of Example 92 except 1-phenylpiperazine was used instead of 1-pyridin-2-ylpiperazine in step 3. LC-MS: 531.2 [M+H]⁺, RT 2.55 min.

### Example 98

### Preparation of 7-[4-(3-chlorophenyl)piperazin-1-yl]-1-{4-[(dimethylamino)methyl]phenyl}-6-fluoro-8-methoxy-4-oxo-1,4-dihydroquinoline-3-carboxylic acid

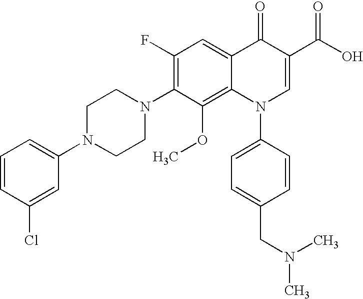

The example was prepared by using the procedure as described for the synthesis of Example 92 except 1-(3-chlorophenyl)piperazine was used instead of 1-pyridin-2-ylpiperazine in step 3. LC-MS: 565.2 [M+H]⁺, RT 2.91 min.

### Example 99

### Preparation of 1-{4-[(dimethylamino)methyl]phenyl}-6-fluoro-8-methoxy-7-[4-(2-methoxyphenyl)piperazin-1-yl]-4-oxo-1,4-dihydroquinoline-3-carboxylic acid

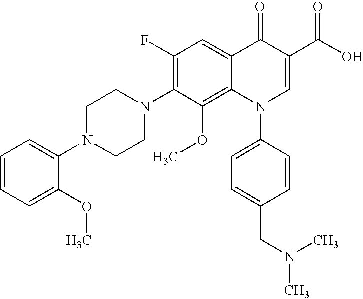

The example was prepared by using the procedure as described for the synthesis of Example 92 except 1-(2-methoxyphenyl)piperazine was used instead of 1-pyridin-2-ylpiperazine in step 3. LC-MS: 561.2 [M+H]⁺, RT 2.36 min.

### Example 100

### Preparation of 1-{4-[(dimethylamino)methyl]phenyl}-6-fluoro-8-methoxy-4-oxo-7-{4-[4-(trifluoromethyl)phenyl]piperazin-1-yl}-1,4-dihydroquinoline-3-carboxylic acid

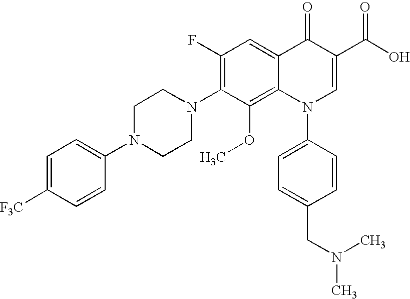

The example was prepared by using the procedure as described for the synthesis of Example 92 except 1-(4-trifluoromethylphenyl)piperazine was used instead of 1-pyridin-2-ylpiperazine in step 3. LC-MS: 599.1 [M+H]⁺, RT 2.99 min.

### Example 101

### Preparation of 7-[4-(2,4-difluorophenyl)piperazin-1-yl]-1-{4-[(dimethylamino)methyl]phenyl}-6-fluoro-8-methoxy-4-oxo-1,4-dihydroquinoline-3-carboxylic acid

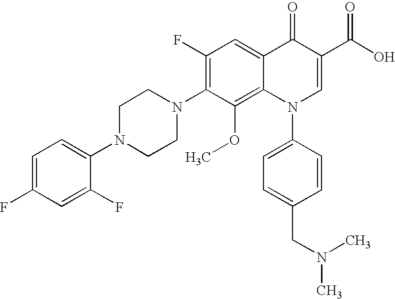

The example was prepared by using the procedure as described for the synthesis of Example 92 except 1-(2,4-difluorophenyl)piperazine was used instead of 1-pyridin-2-ylpiperazine in step 3. LC-MS: 567.8 [M+H]⁺, RT 2.68 nm.

### Example 102

### Preparation of 7-[4-(4-cyanophenyl)piperazin-1-yl]-1-{4-[(dimethylamino)methyl]phenyl}-6-fluoro-8-methoxy-4-oxo-1,4-dihydroquinoline-3-carboxylic acid

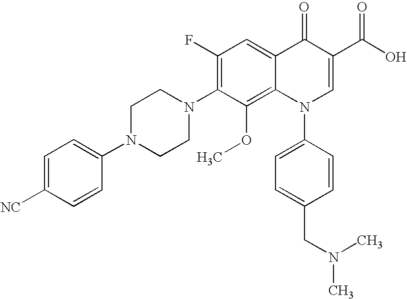

The example was prepared by using the procedure as described for the synthesis of Example 92 except 4-piperazin-1-ylbenzonitrile was used instead of 1-pyridin-2-ylpiperazine in step 3. LC-MS: 556.3 [M+H]⁺, RT 2.55 min.

### Example 103

### Preparation of 7-[4-(4-acetylphenyl)piperazin-1-yl]-1-{4-[(dimethylamino)methyl]phenyl}-6-fluoro-8-methoxy-4-oxo-1,4-dihydroquinoline-3-carboxylic acid

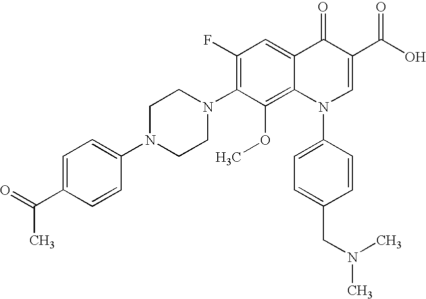

The example was prepared by using the procedure as described for the synthesis of Example 92 except 1-(4-piperazin-1-ylphenyl)ethanone was used instead of 1-pyridin-2-ylpiperazine in step 3. LC-MS: 573.5 [M+H]⁺, RT 2.46 nm.

### Example 104

### Preparation of 1-{4-[(dimethylamino)methyl]phenyl}-6-fluoro-8-methoxy-7-[4-(2-nitrophenyl)piperazin-1-yl]-4-oxo-1,4-dihydroquinoline-3-carboxylic acid

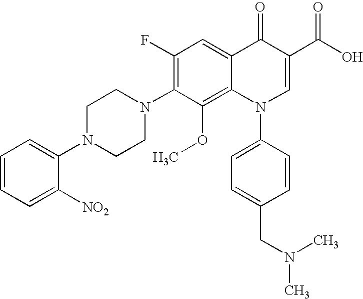

The example was prepared by using the procedure as described for the synthesis of Example 92 except 1-(2-nitrophenyl)piperazine was used instead of 1-pyridin-2-ylpiperazine in step 3. LC-MS: 576.2 [M+H]⁺, RT 2.63 min.

### Example 105

### Preparation of 1-{4-[(diethylamino)methyl]phenyl}-6-fluoro-7-[4-(4-fluorophenyl)piperazin-1-yl]-8-methoxy-4-oxo-1,4-dihydroquinoline-3-carboxylic acid

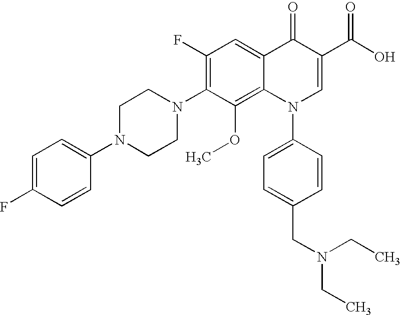

### Step 1: Preparation of ethyl 1-{4-[(diethylamino)methyl]phenyl}-6,7-difluoro-8-methoxy-4-oxo-1,4-dihydroquinoline-3-carboxylate

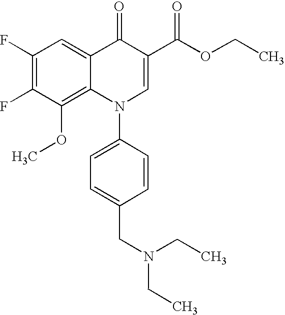

This intermediate was prepared using the procedure as described for the synthesis of Intermediate C except 4-[(diethylamino)methyl]aniline was used instead of 4-pyrrolidin-1-ylmethyl-phenylamine.

### Step 2: Preparation of the Title Compound

The example was then prepared by using procedure as described for the synthesis of Example 72 except ethyl 1-{4-[(diethylamino)methyl]phenyl}-6,7-difluoro-8-methoxy-4-oxo-1,4-dihydroquinoline-3-carboxylate was used instead of Ethyl 6,7-difluoro-8-methoxy-4-oxo-1-[4-(pyrrolidin-1-ylmethyl)phenyl]-1,4-dihydroquinoline-3-carboxylate (Intermediate C) in step 1, and 1-(4-fluorophenyl)piperazine was used instead of 1-pyridin-2-ylpiperazine in step 3. LC-MS: 577.3 [M+H]⁺, RT 3.08 min.

### Example 106

### Preparation of 1-{4-[(diethylamino)methyl]phenyl}-6-fluoro-8-methoxy-4-oxo-7-(4-pyrimidin-2-ylpiperazin-1-yl)-1,4-dihydroquinoline-3-carboxylic acid

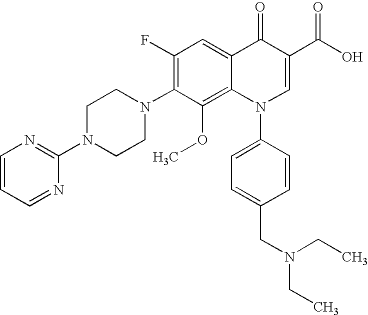

The example was prepared by using the procedure as described for the synthesis of Example 105 except 2-piperazin-1-ylpyrimidine was used instead of 1-(4-fluorophenyl)piperazine in step 3. LC-MS: 561.3 [M+H]⁺, RT 2.83 min.

### Example 107

### Preparation of 1-{4-[(diethylamino)methyl]phenyl}-6-fluoro-8-methoxy-7-[4-(2-nitrophenyl)piperazin-1-yl]-4-oxo-1,4-dihydroquinoline-3-carboxylic acid

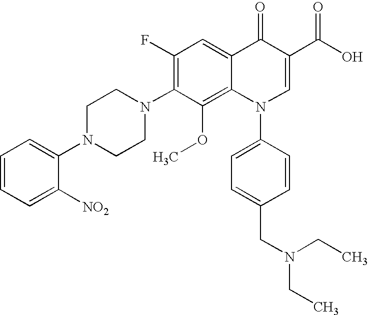

The example was prepared by using the procedure as described for the synthesis of Example 105 except 1-(2-nitrophenyl)piperazine was used instead of 1-(4-fluorophenyl)piperazine in step 3. LC-MS: 561.3 [M+H]⁺, RT 2.83 min. LC-MS: 604-2 [M+H]⁺, RT 3.17 min.

### Example 108

### Preparation of 7-[4-(4-cyanophenyl)piperazin-1-yl]-1-{4-[(diethylamino)methyl]phenyl}-6-fluoro-8-methoxy-4-oxo-1,4-dihydroquinoline-3-carboxylic acid

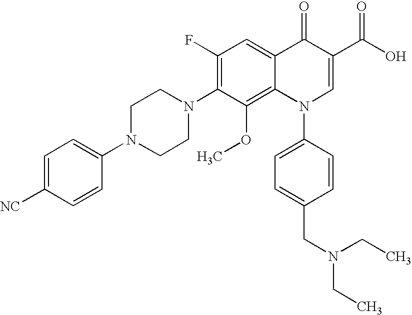

The example was prepared by using the procedure as described for the synthesis of Example 105 except 4-piperazin-1-ylbenzonitrile was used instead of 1-(4-fluorophenyl)piperazine in step 3. LC-MS: 584.3 [M+H]⁺, RT 3.09 min.

### Example 109

### Preparation of 7-[4-(2-chlorophenyl)piperazin-1-yl]-1-{4-[(diethylamino)methyl]phenyl}-6-fluoro-8-methoxy-4-oxo-1,4-dihydroquinoline-3-carboxylic acid

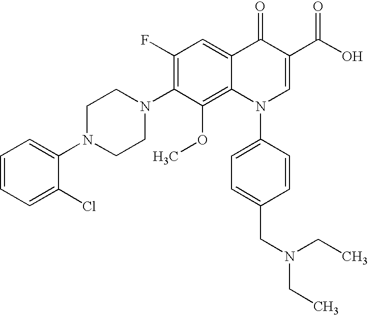

The example was prepared by using the procedure as described for the synthesis of Example 105 except 1-(2-chlorophenyl)piperazine was used instead of 1-(4-fluorophenyl)piperazine in step 3. LC-MS: 593.2[M+H]⁺, RT 3.31 min.

### Example 110

### Preparation of 1-{4-[(diethylamino)methyl]phenyl}-6-fluoro-8-methoxy-7-[4-(2-methoxyphenyl)piperazin-1-yl]-4-oxo-1,4-dihydroquinoline-3-carboxylic acid

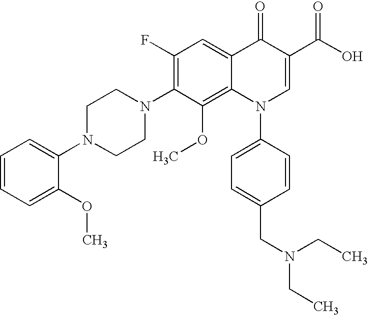

The example was prepared by using the procedure as described for the synthesis of Example 105 except 1-(2-methoxyphenyl)piperazine was used instead of 1-(4-fluorophenyl)piperazine in step 3. LC-MS: 589.3 [M+H]⁺, RT 2.40 min.

### Example 111

### Preparation of 1-{4-[(diethylamino)methyl]phenyl}-7-[4-(2,4-difluorophenyl)piperazin-1-yl]-6-fluoro-8-methoxy-4-oxo-1,4-dihydroquinoline-3-carboxylic acid

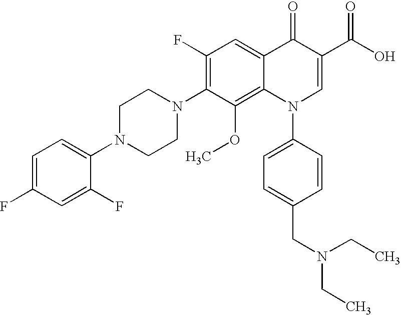

The example was prepared by using the procedure as described for the synthesis of Example 105 except 1-(2,4-difluorophenyl)piperazine was used instead of 1-(4-fluorophenyl)piperazine in step 3. LC-MS: 595.2 [M+H]⁺, RT 2.81 min.

### Example 112

### Preparation of 1-{4-[(diethylamino)methyl]phenyl}-6-fluoro-8-methoxy-4-oxo-7-{4-[4-(trifluoromethyl)phenyl]piperazin-1-yl}-1,4-dihydroquinoline-3-carboxylic acid

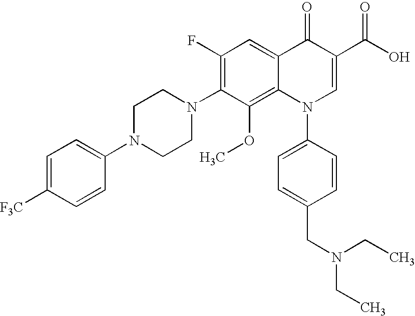

The example was prepared by using the procedure as described for the synthesis of Example 105 except 1-(4-trifluoromethylphenyl)piperazine was used instead of 1-(4-fluorophenyl)piperazine in step 3. LC-MS: 627.2 [M+H]⁺, RT 3.02 min.

### Example 113

### Preparation of 7-[4-(3-cyanopyridin-2-yl)piperazin-1-yl]-1-{4-[(diethylamino)methyl]phenyl}-6-fluoro-8-methoxy-4-oxo-1,4-dihydroquinoline-3-carboxylic acid

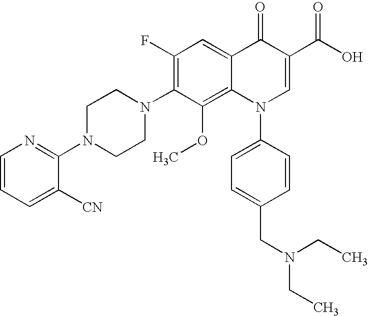

The example was prepared by using the procedure as described for the synthesis of Example 105 except 2-piperazin-1-ylnicotinonitrile was used instead of 1-(4-fluorophenyl)piperazine in step 3. LC-MS: 585.2 [M+H]⁺, RT 2.56 min.

### Example 114

### Preparation of 1-{4-[(diethylamino)methyl]phenyl}-6-fluoro-7-[4-(2-fluorophenyl)piperazin-1-yl]-8-methoxy-4-oxo-1,4-dihydroquinoline-3-carboxylic acid

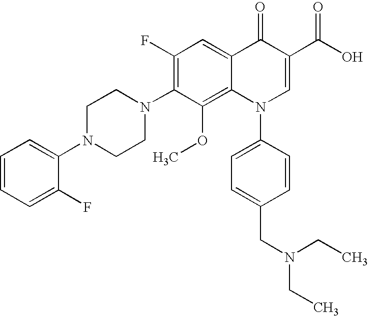

The example was prepared by using the procedure as described for the synthesis of Example 105 except 1-(2-fluorophenyl)piperazine was used instead of 1-(4-fluorophenyl)piperazine in step 3. LC-MS: 577.2 [M+H]⁺, RT 2.79 min.

### Example 115

### Preparation of 7-[4-(3-chlorophenyl)piperazin-1-yl]-1-{4-[(diethylamino)methyl]phenyl}-6-fluoro-8-methoxy-4-oxo-1,4-dihydroquinoline-3-carboxylic acid

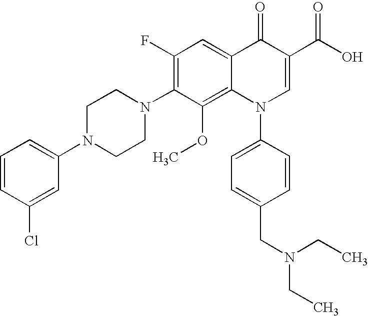

The example was prepared by using the procedure as described for the synthesis of Example 105 except 1-(3-chlorophenyl)piperazine was used instead of 1-(4-fluorophenyl)piperazine in step 3. LC-MS: 593.2 [M+H]⁺, RT 2.90 min.

### Example 116

### Preparation of 7-[4-(4-acetylphenyl)piperazin-1-yl]-1-{4-[(diethylamino)methyl]phenyl}-6-fluoro-8-methoxy-4-oxo-1,4-dihydroquinoline-3-carboxylic acid

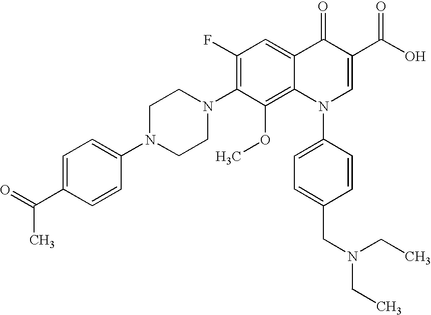

The example was prepared by using the procedure as described for the synthesis of Example 105 except 1-(4-piperazin-1-ylphenyl)ethanone was used instead of 1-(4-fluorophenyl)piperazine in step 3. LC-MS: 601.3 [M+H]⁺, RT 2.62 min.

### Example 117

### Preparation of 7-[4-(2-cyanophenyl)piperazin-1-yl]-1-{4-[(diethylamino)methyl]phenyl}-6-fluoro-8-methoxy-4-oxo-1,4-dihydroquinoline-3-carboxylic acid

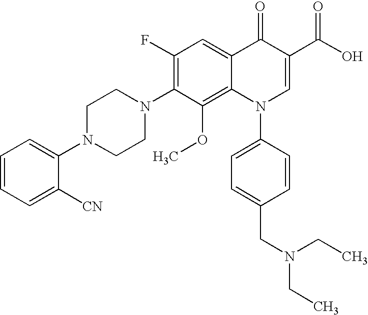

The example was prepared by using the procedure as described for the synthesis of Example 105 except 2-piperazin-1-ylbenzonitrile was used instead of 1-(4-fluorophenyl)piperazine in step 3. LC-MS: 584.3 [M+H]⁺, RT 2.60 min

### Example 118

### Preparation of 7-[4-(4-chlorophenyl)piperazin-1-yl]-1-{4-[(diethylamino)methyl]phenyl}-6-fluoro-8-methoxy-4-oxo-1,4-dihydroquinoline-3-carboxylic acid

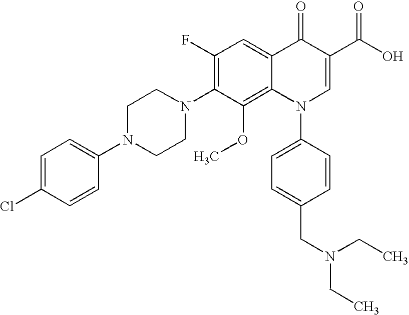

The example was prepared by using the procedure as described for the synthesis of Example 105 except 1-(4-chlorophenyl)piperazine was used instead of 1-(4-fluorophenyl)piperazine in step 3. LC-MS: 593.4 [M+H]⁺, RT 2.82 min.

### Example 119

### Preparation of 7-[4-(3-chlorophenyl)piperazin-1-yl]-6-fluoro-4-oxo-1-[4-(pyrrolidin-1-ylmethyl)phenyl]-8-(trifluoromethoxy)-1,4-dihydroquinoline-3-carboxylic acid

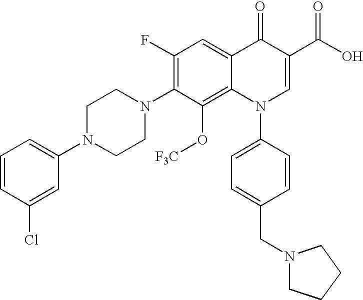

### Step 1: Synthesis of ethyl 7-[4-(3-chlorophenyl)piperazin-1-yl]-6-fluoro-4-oxo-1-[4-(pyrrolidin-1-ylmethyl)phenyl]-8-(trifluoromethoxy)-1,4-dihydroquinoline-3-carboxylate

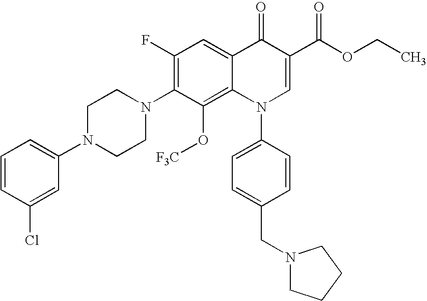

Ethyl 6,7-difluoro-4-oxo-1-[4-(pyrrolidin-1-ylmethyl)phenyl]-8-(trifluoromethoxy)-1,4-dihydroquinoline-3-carboxylate (Intermediate E, 100 mg, 0.20 mmol), DABCO (0.16 mL, 1.0 mmol, 5 equiv.) and 1-(3-chlorophenyl)piperazine (138 mg, 0.71 mmol, 3.5 equiv.) were added to acetonitrile (3.0 mL) in a 40 mL vial (95 mm×28 mm). The vial was sealed with a screw cap containing a septum and placed on a rotary shaker at 100° C. for 5 d. The mixture was cooled to room temperature and the precipitate was collected by filtration, rinsed with methanol (approx. 2 mL), then dried to give 40 mg (29%) of ethyl 7-[4-(3-chlorophenyl)piperazin-1-yl]-6-fluoro-4-oxo-1-[4-(pyrrolidin-1-ylmethyl)phenyl]-8-(trifluoromethoxy)-1,4-dihydroquinoline-3-carboxylate. ¹H NMR (DMSO-d₆) δ 8.33 (s, 1H), 7.95 (d, 1H), 7.53 (d, 1H), 7.46 (d, 1H), 7.21 (t, 1H), 6.98 (t, 1H), 6.92 (dd, 1H), 6.80 (dd, 1H), 4.21 (dd, 2H), 3.67 (br s, 2H), 3.34-3.36 (m, 10H), 2.46-2.49 (m, 2H), 1.71-1.74 (m, 4H), 1.26 (t, 3H).

### Step 2: Synthesis of Title Compound

Ethyl 7-[4-(3-chlorophenyl)piperazin-1-yl]-6-fluoro-4-oxo-1-[4-(pyrrolidin-1-ylmethyl)phenyl]-8-(trifluoromethoxy)-1,4-dihydroquinoline-3-carboxylate (25 mg, 0.037 mmol) was taken up in a mixture of iso-propyl alcohol/HCl (conc.)/H₂O (8/2/1) (5 mL) and heated to reflux for 2 h. The solution was cooled to room temperature and the solvent was removed in vacuo to give 23 mg (89%) of 7-[4-(3-chlorophenyl)piperazin-1-yl]-6-fluoro-4-oxo-1-[4-(pyrrolidin-1-ylmethyl)phenyl]-8-(trifluoromethoxy)-1,4-dihydroquinoline-3-carboxylic acid as the hydrochloride salt. ¹H NMR (DMSO-d₆) δ 10.67-10.69 (m, 1H), 8.56 (d, 1H), 8.12 (d, 1H), 7.75-7.80 (m, 4H), 7.21 (t, 1H), 6.97-6.99 (m, 1H), 6.92 (m, 1H), 6.70 (dd, 1H), 4.44 (d, 2H), 3.73-3.44 (m, 10H), 3.00-3.07 (m, 2H), 1.96-2.04 (m, 2H), 1.84-1.90 (m, 2H); LC-MS RT 2.96 min; [M+H]⁺ 645.7.

### Example 120

### Preparation of 6-fluoro-7-[4-(6-methylpyridin-2-yl)piperazin-1-yl]-4-oxo-1-[4-(pyrrolidin-1-ylmethyl)phenyl]-8-(trifluoromethoxy)-1,4-dihydroquinoline-3-carboxylic acid

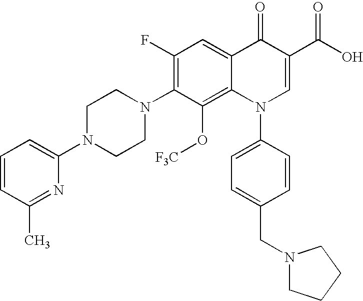

The example was prepared using a similar protocol as Example 119, using 1-(3-methylpyridin-2-yl)piperazine instead of 1-(3-chlorophenyl)piperazine in step 1. LC-MS: 626.2 [M+H]⁺, RT 2.96 min.

### Example 121

### Preparation of 6-fluoro-7-[4-(4-fluorophenyl)piperazin-1-yl]-4-oxo-1-[4-(pyrrolidin-1-ylmethyl)phenyl]-8-(trifluoromethoxy)-1,4-dihydroquinoline-3-carboxylic acid

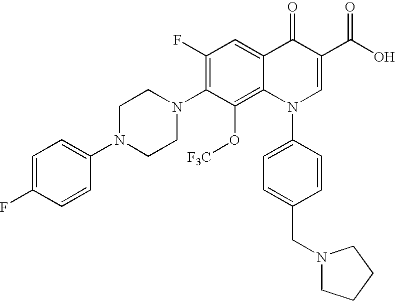

The example was prepared using a similar protocol as Example 119, using 1-(4-fluorophenyl)piperazine instead of 1-(3-chlorophenyl)piperazine in step 1. LC-MS: 629.2 [M+H]⁺, RT 2.11 min.

### Example 122

### Preparation of 6-fluoro-7-[4-(3-methylpyridin-2-yl)-piperazin-1-yl]-4-oxo-1-[4-(pyrrolidin-1-ylmethyl)phenyl]-8-(trifluoromethoxy)-1,4-dihydroquinoline-3-carboxylic acid

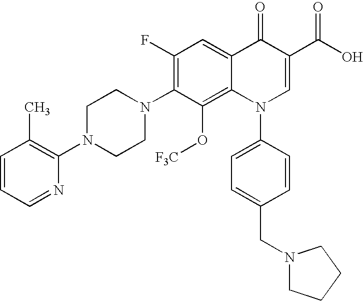

The example was prepared using a similar protocol as Example 119, using 1-(6-methylpyridin-2-yl)piperazine instead of 1-(3-chlorophenyl)piperazine in step 1. LC-MS: 626.2 [M+H]⁺, RT 2.27 min.

### Example 123

### Preparation of 6-fluoro-4-oxo-7-(4-pyrimidin-2-yl-piperazin-1-yl)-1-[4-(pyrrolidin-1-ylmethyl)phenyl]-8-(trifluoromethoxy)-1,4-dihydroquinoline-3-carboxylic acid

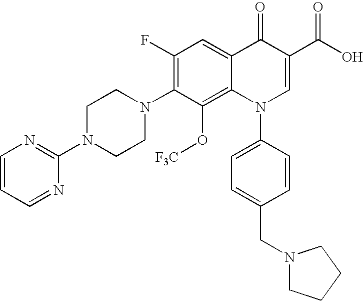

The example was prepared using a similar protocol as Example 119, using 2-piperazin-1-ylpyrimidine instead of 1-(3-chlorophenyl)piperazine in step 1. LC-MS: 613.7 [M+H]⁺, RT 2.45 min.

### Example 124

### Preparation of 6-fluoro-4-oxo-7-(4-pyridin-2-yl-piperazin-1-yl)-1-[4-(pyrrolidin-1-ylmethyl)phenyl]-8-(trifluoromethoxy)-1,4-dihydroquinoline-3-carboxylic acid

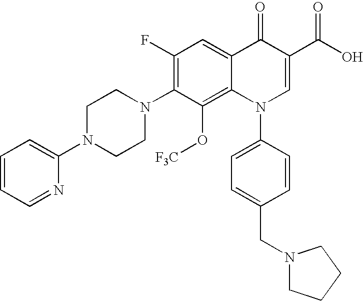

The example was prepared using a similar protocol as Example 119, using 1-pyridin-2-ylpiperazine instead of 1-(3-chlorophenyl)piperazine in step 1. ¹H NMR(CD₃OD): δ 8.73 (s, 1H), 8.17 (d, 1H), 8.09 (ddd, 1H), 7.99 (dd, 1H), 7.84 (d, 1H), 7.72 (d, 1H), 7.46 (d, 1H), 7.05 (t, 1H), 4.56 (s, 2H), 3.87-3.89 (m, 4H), 3.53-3.55 (m, 6H), 3.24-3.28 (m, 2H), 2.22-2.56 (m, 2H), 2.08-2.11 (m, 2H); LC-MS RT 1.94 min; [M+H]⁺ 612.5.

### Example 125

### Preparation of 7-[4-(3-cyanopyridin-2-yl)piperazin-1-yl]-6-fluoro-4-oxo-1-[4-(pyrrolidin-1-ylmethyl)phenyl]-8-(trifluoromethoxy)-1,4-dihydroquinoline-3-carboxylic acid

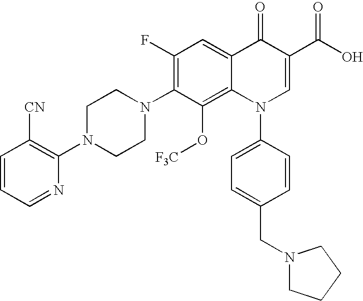

The example was prepared using a similar protocol as Example 119, using 2-piperazin-1-ylnicotinonitrile instead of 1-(3-chlorophenyl)piperazine in step 1. LC-MS: 637.4 [M+H]⁺, RT 2.60 min.

### Example 126

### Preparation of 1-{4-[(dimethylamino)methyl]phenyl}-6-fluoro-4-oxo-7-(4-pyridin-2-ylpiperazin-1-yl)-8-(trifluoromethoxy)-1,4-dihydroquinoline-3-carboxylic acid

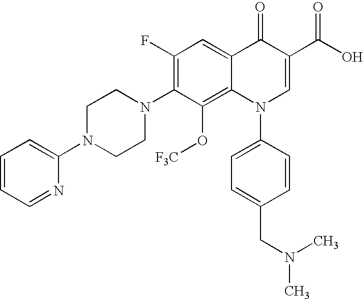

### Step 1: Synthesis of ethyl 1-{4-[(dimethylamino)methyl]phenyl}-6,7-difluoro-4-oxo-8-(trifluoromethoxy)-1,4-dihydroquinoline-3-carboxylate

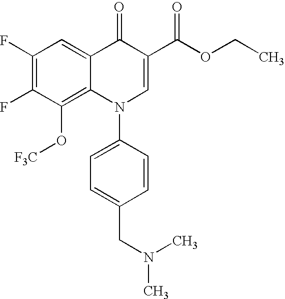

This intermediate was prepared using the procedure as described for synthesis of Intermediate E except 4-[(dimethylamino)methyl]aniline was used instead of 4-pyrrolidin-1-ylmethyl-phenylamine in step 3.

### Step 2: Preparation of the Title Compound

The title compound was prepared using similar procedure as described for the synthesis of Example 119, using ethyl 1-{4-[(dimethylamino)methyl]phenyl}-6,7-difluoro-4-oxo-8-(trifluoromethoxy)-1,4-dihydroquinoline-3-carboxylate instead of ethyl 6,7-difluoro-4-oxo-1-[4-(pyrrolidin-1-ylmethyl)phenyl]-8-(trifluoromethoxy)-1,4-dihydroquinoline-3-carboxylate and 1-pyridin-2-ylpiperazine instead of 1-(3-chlorophenyl)piperazine in step 1. LC-MS: 586.3 [M+H]⁺, RT 1.92 min.

### Example 127

### Preparation of 1-{4-[(dimethylamino)methyl]phenyl}-6-fluoro-7-[4-(6-methylpyridin-2-yl)piperazin-1-yl]-4-oxo-8-(trifluoromethoxy)-1,4-dihydroquinoline-3-carboxylic acid

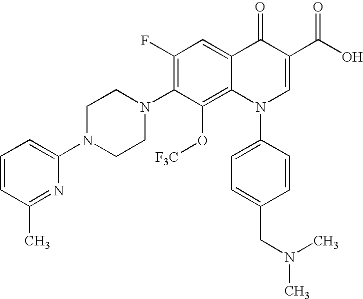

The example was prepared using a similar protocol as Example 126, using 1-(3-methylpyridin-2-yl)piperazine instead of 1-pyridin-2-ylpiperazine. LC-MS: 600.2 [M+H]⁺, RT 2.09 min.

### Example 128

### Preparation of 1-{4-[(dimethylamino)methyl]phenyl}-6-fluoro-4-oxo-7-(4-pyrimidin-2-ylpiperazin-1-yl)-8-(trifluoromethoxy)-1,4-dihydroquinoline-3-carboxylic acid

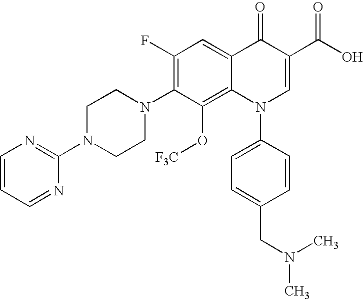

The example was prepared using a similar protocol as Example 126, using 2-piperazin-1-ylpyrimidine instead of 1-pyridin-2-ylpiperazine. ¹H NMR (CD₃OD): δ 8.73 (s, 1H), 8.51 (d, 2H), 8.20 (d, 1H), 7.76 (dd, 4H), 6.89)t, 1H), 4.48 (s, 2H), 3.99-4.01 (m, 4H), 3.44-3.46 (m, 4H), 2.93 (s, 6H); LC-MS: 587.2 [M+H]⁺, RT 2.40 min.

### Example 129

### Preparation of 1-{4-[(dimethylamino)methyl]phenyl}-6-fluoro-7-[4-(3-methylpyridin-2-yl)piperazin-1-yl]-4-oxo-8-(trifluoromethoxy)-1,4-dihydroquinoline-3-carboxylic acid

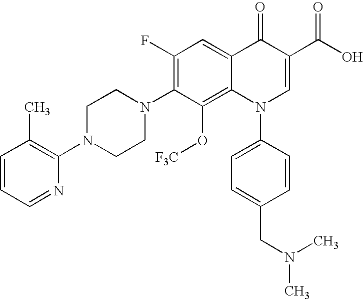

The example was prepared using a similar protocol as Example 126, using 1-(6-methylpyridin-2-yl)piperazine instead of 1-pyridin-2-ylpiperazine. LC-MS: 600.2 [M+H]⁺, RT 2.24 min.

### Example 130

### Preparation of 7-[4-(3-chlorophenyl)piperazin-1-yl]-1-{4-[(dimethylamino)methyl]phenyl}-6-fluoro-4-oxo-8-(trifluoromethoxy)-1,4-dihydroquinoline-3-carboxylic acid

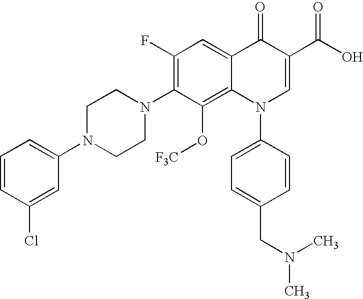

The example was prepared using a similar protocol as Example 126, using 1-(3-chlorophenyl)piperazine instead of 1-pyridin-2-ylpiperazine. LC-MS: 619.2 [M+H]⁺, RT 2.92 min.

### Example 131

### Preparation of 1-{4-[(dimethylamino)methyl]phenyl}-6-fluoro-7-[4-(4-fluorophenyl)piperazin-1-yl]-4-oxo-8-(trifluoromethoxy)-1,4-dihydroquinoline-3-carboxylic acid

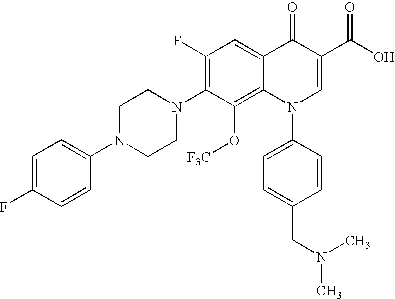

The example was prepared using a similar protocol as Example 126, using 1-(4-fluorophenyl)piperazine instead of 1-pyridin-2-ylpiperazine. LC-MS: 603.2 [M+H]⁺, RT 2.79 min.

### Example 132

### Preparation of 1-{4-[(diethylamino)methyl]phenyl}-6-fluoro-4-oxo-7-(4-pyridin-2-ylpiperazin-1-yl)-8-(trifluoromethoxy)-1,4-dihydroquinoline-3-carboxylic acid

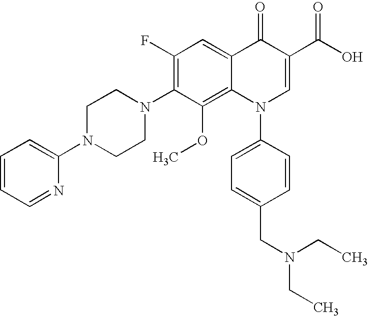

### Step 1: Synthesis of ethyl 1-{4-[(diethylamino)methyl]phenyl}-6,7-difluoro-4-oxo-8-(trifluoromethoxy)-1,4-dihydroquinoline-3-carboxylate

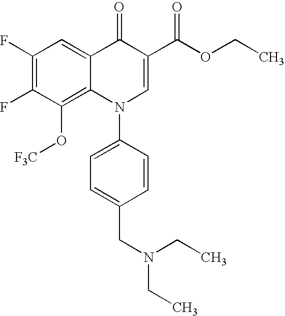

This intermediate was prepared using the procedure as described for synthesis of Intermediate E except 4-[(diethylamino)methyl]aniline was used instead of 4-pyrrolidin-1-ylmethyl-phenylamine in step 3.

### Step 2: Preparation of the Title Compound

The title compound was prepared using similar procedure as described for the synthesis of Example 119, using ethyl 1-{4-[(diethylamino)methyl]phenyl}-6,7-difluoro-4-oxo-8-(trifluoromethoxy)-1,4-dihydroquinoline-3-carboxylate instead of ethyl 6,7-difluoro-4-oxo-1-[4-(pyrrolidin-1-ylmethyl)phenyl]-8-(trifluoro-methoxy)-1,4-dihydroquinoline-3-carboxylate. LC-MS: 614.8 [M+H]⁺, RT 2.08 min.

### Example 133

### Preparation of ethyl 1-{4-[(diethylamino)methyl]-phenyl}-6-fluoro-4-oxo-7-(4-pyrimidin-2-ylpiperazin-1-yl)-8-(trifluoromethoxy)-1,4-dihydroquinoline-3-carboxylate

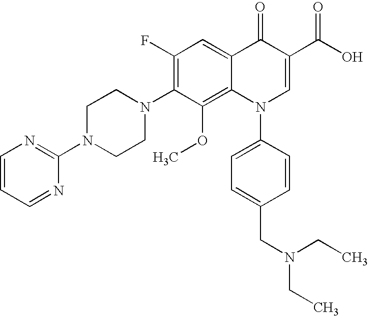

The example was prepared using a similar protocol as Example 132, using 2-piperazin-1-ylpyrimidine instead of 1-pyridin-2-ylpiperazine. LC-MS: 615.8 [M+H]⁺, RT 2.58 min.

### Example 134

### Preparation of 7-[4-(5-chloro-2-methylphenyl)piperazin-1-yl]-1-{4-[(diethylamino)methyl]phenyl}-6-fluoro-4-oxo-8-(trifluoromethoxy)-1,4-dihydroquinoline-3-carboxylic acid

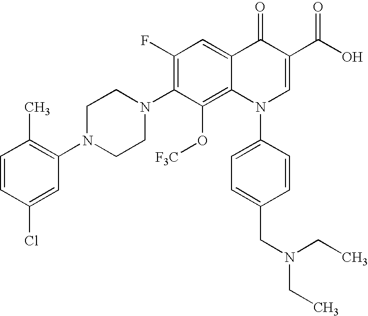

The example was prepared using a similar protocol as Example 132, using 1-(5-chloro-2-methylphenyl)piperazine instead of 1-pyridin-2-ylpiperazine. LC-MS: 661.8 [M+H]⁺, RT 3.27 min.

### Example 135

### Preparation of 1-{4-[(diethylamino)methyl]phenyl}-6-fluoro-7-[4-(2-fluorophenyl)piperazin-1-yl]-4-oxo-8-(trifluoromethoxy)-1,4-dihydroquinoline-3-carboxylic acid

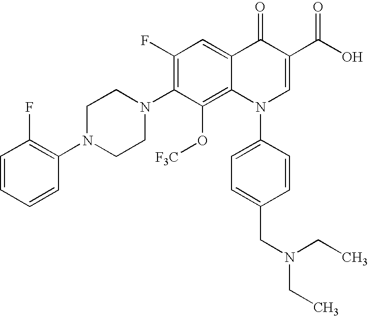

The example was prepared using a similar protocol as Example 132, using 1-(2-fluorophenyl)piperazine instead of 1-pyridin-2-ylpiperazine. LC-MS: 631.2 [M+H]⁺, RT 2.94 min.

### Example 136

### Preparation of 7-[4-(3-chlorophenyl)piperazin-1-yl]-1-{4-[(diethylamino)methyl]phenyl}-6-fluoro-4-oxo-8-(trifluoromethoxy)-1,4-dihydroquinoline-3-carboxylic acid

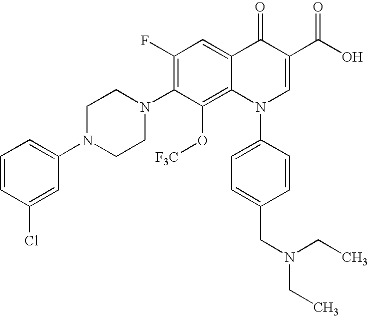

The example was prepared using a similar protocol as Example 132, using 1-(3-chlorophenyl)piperazine instead of 1-pyridin-2-ylpiperazine. LC-MS: 647.2 [M+H]⁺, RT 3.06 min.

### Example 137

### Preparation of 1-{4-[(diethylamino)methyl]phenyl}-7-[4-(2,4-difluorophenyl)piperazin-1-yl]-6-fluoro-4-oxo-8-(trifluoromethoxy)-1,4-dihydroquinoline-3-carboxylic acid

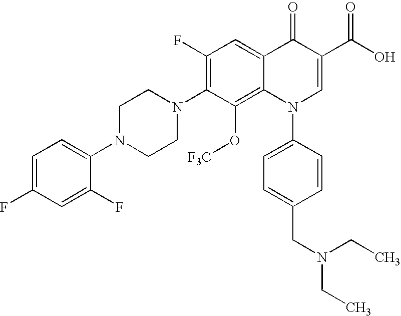

The example was prepared using a similar protocol as Example 132, using 1-(2,4-difluorophenyl)piperazine instead of 1-pyridin-2-ylpiperazine. LC-MS: 649.6 [M+H]⁺, RT 2.94 min.

### Example 138

### Preparation of 1-{4-[(diethylamino)methyl]phenyl}-6-fluoro-4-oxo-7-(4-phenylpiperazin-1-yl)-8-(trifluoromethoxy)-1,4-dihydroquinoline-3-carboxylic acid

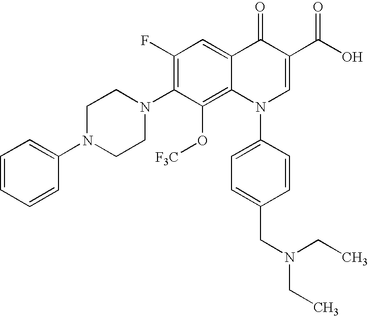

The example was prepared using a similar protocol as Example 132, using 1-phenylpiperazine instead of 1-pyridin-2-ylpiperazine. LC-MS: 613.5 [M+H]⁺, RT 2.68 min.

### Example 139

### Preparation of 6-fluoro-1-(4-{[(2S)-2-methylpiperidin-1-yl]methyl}phenyl)-4-oxo-7-(4-pyrimidin-2-ylpiperazin-1-yl)-8-(trifluoromethoxy)-1,4-dihydroquinoline-3-carboxylic acid

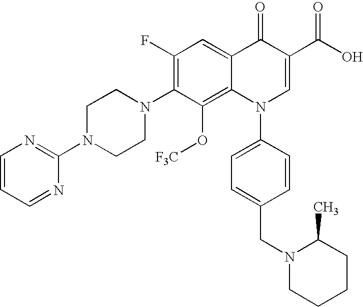

### Step 1: Preparation of 4-{[(2S)-2-methylpiperidin-1-yl]methyl}aniline

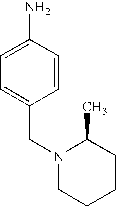

This intermediate was prepared using the procedure as described for the synthesis of Intermediate A except (2S)-2-methylpiperidine was used instead of pyrrolidine in the step 1.

### Step 2: Preparation of ethyl 6,7-difluoro-1-(4-{[(2S)-2-methylpiperidin-1-yl]methyl}phenyl)-4-oxo-8-(trifluoromethoxy)-1,4-dihydroquinoline-3-carboxylate

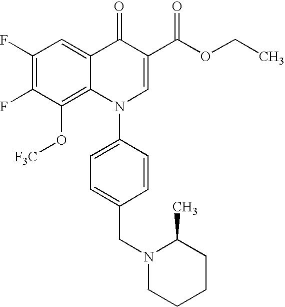

This intermediate was prepared using the procedure as described for synthesis of Intermediate E, except 4-{[(2S)-2-methylpiperidin-1-yl]methyl}aniline was used instead of 4-pyrrolidin-1-ylmethyl-phenylamine in step 3.

### Step 3: Preparation of the Title Compound

The title compound was prepared using similar procedure as described for the synthesis of Example 119, using ethyl 6,7-difluoro-1-(4-{[(2S)-2-methylpiperidin-1-yl]methyl}phenyl)-4-oxo-8-(trifluoromethoxy)-1,4-dihydro-quinoline-3-carboxylate instead of ethyl 6,7-difluoro-4-oxo-1-[4-(pyrrolidin-1-ylmethyl)phenyl]-8-(trifluoromethoxy)-1,4-dihydroquinoline-3-carboxylate and using 2-piperazin-1-ylpyrimidine instead of 1-pyridin-2-ylpiperazine in step 1. LC-MS: 604.3 [M+H]⁺, RT 2.53 min.

### Example 140

### Preparation of 6-fluoro-4-oxo-1-[3-(piperidin-1-yl-methyl)phenyl]-7-(4-pyridin-2-ylpiperazin-1-yl)-8-(trifluoromethoxy)-1,4-dihydroquinoline-3-carboxylic acid

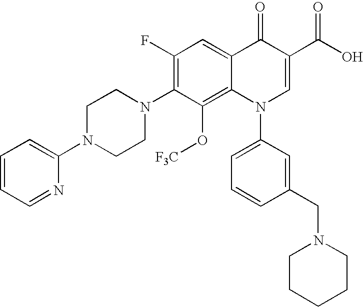

### Step 1: Preparation of [3-(piperidin-1-ylmethyl)phenyl]amine

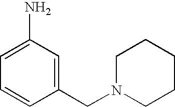

Piperidine (5.26 mL, 53 mmol, 2.3 equiv.) was added to a solution of 1-(bromomethyl)-3-nitrobenzene (5.0 g, 23 mmol) in THF (100 mL) at room temperature. The mixture was stirred for 3 h then the piperidine hydrobromide was filtered off and the solvent was removed from the filtrate in vacuo. The oil was dissolved in toluene (approx. 20 mL) then concentrated in vacuo three times to remove excess piperidine. The oil was then dissolved in EtOAc (100 mL) and the flask was purged with nitrogen. Raney Nickel (slurry, 200 mg) was added to the flask and the flask was purged 3 times with hydrogen. The reaction stirred for 2 d under hydrogen. The catalyst was filtered off and the solvent was removed in vacuo to give 4 g (91%) of [3-(piperidin-1-ylmethyl)phenyl]amine.

### Step 2: Preparation of ethyl-3{[3-(piperidin-1-ylmethyl)phenyl]amino}-2-[2,4,5-trifluoro-3-(trifluoromethoxy)benzoyl]acrylate

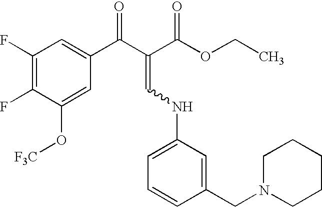

Ethyl-3-ethoxy-2-[2,4,5-trifluoro-3-(trifluoromethoxy)benzoyl]acrylate (2.0 g, 5.2 mmol) was dissolved in EtOH (abs., 50.0 mL) then cooled to 0° C. [3-(Piperidin-1-ylmethyl)phenyl]amine (985 mg, 5.2 mmol) was added and the mixture was allowed to warm to room temperature and stir for 2 h. LC-MS analysis showed the reaction was complete. Solvent was removed in vacuo and the ethyl-3{[3-(piperidin-1-ylmethyl)phenyl]amino}-2-[2,4,5-trifluoro-3-(trifluoro-methoxy)benzoyl]acrylate was taken on without further purification. LC-MS RT 2.88 min; [M+H]⁺ 531.1.

### Step 3: Preparation of ethyl 6,7-difluoro-4-oxo-1-[3-(piperidin-1-ylmethyl)phenyl]-8-(trifluoromethoxy)-1,4-dihydroquinoline-3-carboxylate

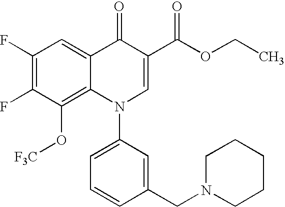

### Ethyl-3{[3-(piperidin-1-ylmethyl)phenyl]amino}-2-[2,4,5-trifluoro-3-(trifluoromethoxy)benzoyl]acrylate (approx 5.2 mmol) was dissolved in THF (10 mL) and K₂CO₃ (2.15 g, 15.5 mmol, 3 equiv.) and 18-crown-6 (957 mg, 1.55 mmol, 0.3 equiv.) were added. The mixture was heated to reflux for 2 h; LC-MS showed the starting material was gone. The reaction was cooled to room temperature, filtered and the solids were rinsed with THF. The solvent was removed from the filtrate in vacuo and hexanes were added to the oil and the mixture was stirred 18 h. The precipitate was filtered and dried in a drying oven to give 1200 mg (45%, 2 steps) of ethyl 6,7-difluoro-4-oxo-1-[3-(piperidin-1-ylmethyl)phenyl]-8-(trifluoro-methoxy)-1,4-dihydroquinoline-3-carboxylate as an off-white solid. LC-MS RT 2.24 min; [M+H]⁺ 512.0.

The ethyl-3 {[3-(piperidin-1-ylmethyl)phenyl]amino}-2-[2,4,5-trifluoro-3-(trifluoromethoxy)benzoyl]acrylate (approx 5.2 mmol) was dissolved in THF (10 mL) and K₂CO₃ (2.15 g, 15.5 mmol, 3 equiv.) and 18-crown-6 (957 mg, 1.55 mmol, 0.3 equiv.) were added. The mixture was heated to reflux for 2 h; LC-MS showed the starting material was gone. The reaction was cooled to room temperature, filtered and the solids were rinsed with THF. The solvent was removed from the filtrate in vacuo and hexanes were added to the oil and the mixture was stirred 18 h. The precipitate was filtered and dried in a drying oven to give 1200 mg (45%, 2 steps) of ethyl 6,7-difluoro-4-oxo-1-[3-(piperidin-1-ylmethyl)phenyl]-8-(trifluoro-methoxy)-1,4-dihydroquinoline-3-carboxylate as an off-white solid. LC-MS RT 2.24 min; [M+H]⁺ 512.0.

### Step 4: Synthesis of ethyl 6-fluoro-4-oxo-1-[3-(piperidin-1-ylmethyl)phenyl]-7-(4-pyridin-2-ylpiperazin-1-yl)-8-(trifluoromethoxy)-1,4-dihydroquinoline-3-carboxylate

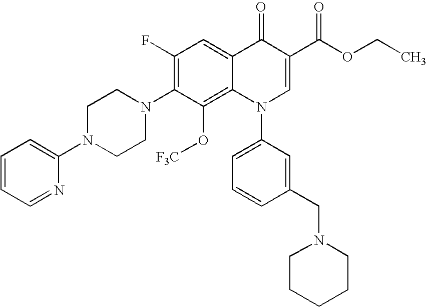

This was synthesized using the step 3 product and 1-pyridin-2-ylpiperazine following the procedure as described in step 1 of Example 119. ¹H NMR (acetone-d₆): δ 8.41 (s, 1H), 8.13 (ddd, 1H), 7.99 (d, 1H), 7.47-7.56 (m, 5H), 6.83 (d, 1H), 6.65 (ddd, 1H), 4.26 (dd, 2H), 3.67 (t, 4H), 3.54 (s, 2H), 3.36-3.37 (m, 4H), 2.42-2.44 (m, 4H), 1.55-1.60 (m, 4H), 1.47-1.49 (m, 2H), 1.31 (t, 3H); LC-MS: 654.9 [M+H]⁺, RT 1.97 min.

### Step 5: Synthesis of Title Compound

Hydrolysis of the step 4 product was carried out by following the procedure as described in step 2 of Example 119. ¹H NMR (CD₃OD): δ 8.76 (s, 1H), 8.22 (d, 1H), 8.08 (ddd, 1H), 7.98 (ddd, 1H), 7.87 (br s, 1H), 7.72-7.74 (m, 3H), 7.44 (d, 1H), 7.05 (m, 1H), 4.44 (dd, 2H), 3.85 (t, 4H), 3.48-3.63 (m, 6H), 3.01-3.08 (m, 2H), 1.96-2.00 (m, 2H), 1.79-1.91 (m, 3H), 1.54-1.58 (m, 1H); LC-MS: 626.3 [M+H]⁺, RT 2.07 min.

### Example 141

### Preparation of 6-fluoro-7-[4-(4-fluorophenyl)piperazin-1-yl]-4-oxo-1-[3-(piperidin-1-ylmethyl)phenyl]-8-(trifluoromethoxy)-1,4-dihydroquinoline-3-carboxylic acid

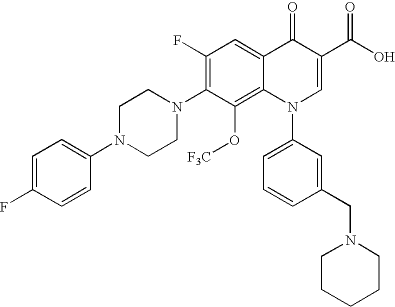

The example was prepared using a similar protocol as Example 140, using 1-(4-fluorophenyl)piperazine instead of 1-pyridin-2-ylpiperazine in step 4. LC-MS: 643.3 [M+H]⁺, RT 2.86 min.

### Example 142

### Preparation of 7-[4-(2,4-difluorophenyl)piperazin-1-yl]-6-fluoro-4-oxo-1-[3-(piperidin-1-ylmethyl)phenyl]-8-(trifluoromethoxy)-1,4-dihydroquinoline-3-carboxylic acid (6)

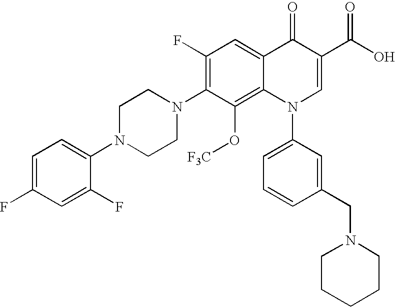

The example was prepared using similar protocol as Example 140, using 1-(2,4-difluorophenyl)piperazine instead of 1-pyridin-2-ylpiperazine in step 4. LC-MS: 661.3 [M+H]⁺, RT 2.98 min.

### Example 143

### Preparation of 6-fluoro-4-oxo-1-[3-(piperidin-1-yl-methyl)phenyl]-7-(4-pyrimidin-2-ylpiperazin-1-yl)-8-(trifluoromethoxy)-1,4-dihydroquinoline-3-carboxylic acid

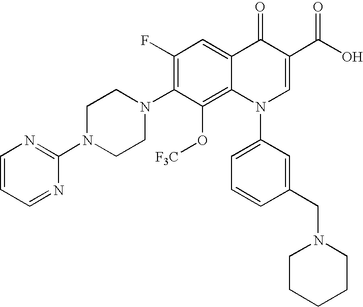

The example was prepared using a similar protocol as Example 140, using 2-piperazin-1-ylpyrimidine instead of 1-pyridin-2-ylpiperazine in step 4. LC-MS: 627.8 [M+H]⁺, RT 2.46 min.

### Example 144

### Preparation of 6-fluoro-7-[4-(2-fluorophenyl)piperazin-1-yl]-4-oxo-1-[3-(piperidin-1-ylmethyl)phenyl]-8-(trifluoromethoxy)-1,4-dihydroquinoline-3-carboxylic acid

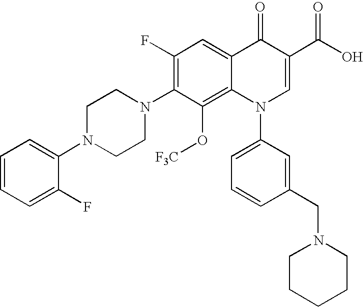

The example was prepared using a similar protocol as Example 140, using 1-(2-fluorophenyl)piperazine instead of 1-pyridin-2-ylpiperazine in step 4. LC-MS: 643.8 [M+H]⁺, RT 2.82 min.

### Example 145

### Preparation of 7-[4-(3-chlorophenyl)piperazin-1-yl]-6-fluoro-4-oxo-1-[3-(piperidin-1-ylmethyl)phenyl]-8-(trifluoromethoxy)-1,4-dihydroquinoline-3-carboxylic acid

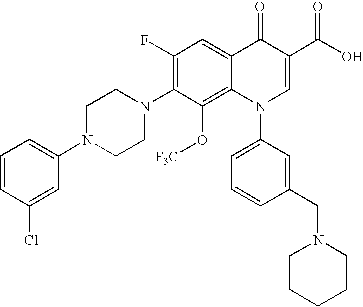

The example was prepared using a similar protocol as Example 140, using 1-(3-chlorophenyl)piperazine instead of 1-pyridin-2-ylpiperazine in step 4. LC-MS: 660 [M+H]⁺, RT 2.94 min.

### Example 146

### Preparation of 8-[chloro(difluoro)methoxy]-6-fluoro-4-oxo-7-(4-pyridin-2-ylpiperazin-1-yl)-1-[4-(pyrrolidin-1-ylmethyl)phenyl]-1,4-dihydroquinoline-3-carboxylic acid

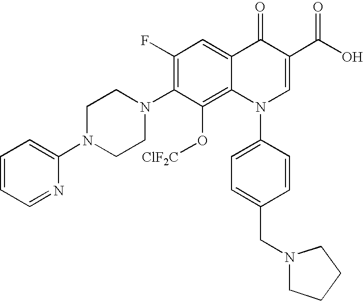

### Step 1: Preparation of ethyl 8-[chloro(difluoro)methoxy]-6-fluoro-4-oxo-7-(4-pyridin-2-ylpiperazin-1-yl)-1-[4-(pyrrolidin-1-ylmethyl)phenyl]-1,4-dihydro-quinoline-3-carboxylate

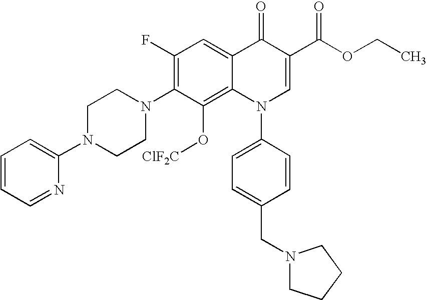

A solution of ethyl 8-[chloro(difluoro)methoxy]-6,7-difluoro-4-oxo-1-[4-(pyrrolidin-1-ylmethyl)phenyl]-1,4-dihydroquinoline-3-carboxylate (Intermediate D, 0.78 g, 1.52 mmol), 1-(2-pyridyl)piperazine (0.74 g, 4.56 mmol) and DIEA (0.59 g, 4.56 mmol) in DMSO (50 mL) was heated at 95 C for 18 h. The reaction was monitored by LC-MS until the starting material ethyl 8-[chloro(difluoro)methoxy]-6,7-difluoro-4-oxo-1-[4-(pyrrolidin-1-ylmethyl)-phenyl]-1,4-dihydroquinoline-3-carboxylate was all consumed in 18 h. After removal of the solvent to dryness, the resulting solid was purified by silica gel column eluted with methanol/dichloromethane (3/97). The pure product was obtained as yellow foam (0.6 g, 60% yield). LC-MS 656 [M+H]⁺, RT 2.10 min.

### Step 2: Preparation of the Title Compound

A solution of ethyl 8-[chloro(difluoro)methoxy]-6-fluoro-4-oxo-7-(4-pyridin-2-ylpiperazin-1-yl)-1-[4-(pyrrolidin-1-ylmethyl)phenyl]-1,4-dihydro-quinoline-3-carboxylate (0.6 g, 0.9 mmol) in IPA/H₂O/HCl (100:10:20) was heated at 90 C overnight. LC-MS showed no starting material left. After removal of the solvent, the crude product was purified by recrystallization from IPA/methanol (100/1). The pure product was obtained as yellow solid (320 mg, 50% yield). LC-MS 628 [M+H]⁺, RT 2.10 min. ¹H NMR (methanol-d4): 8.73(s, 1H), 8.21(d, 1H), 8.10-7.95(m, 2H), 7.80(d, 2H), 7.72(d, 2H), 7.41(d, 1H), 7.03(t, 1H), 4.55(s, 2H), 3.84 (s, 4H), 3.58(s, 6H), 3.28(s, 2H), 2.3(s, 2H), 2.10(s, 2H). LC-MS: 628 [M+H]⁺, RT 1.92 min.

### Example 147

### Preparation of 8-[chloro(difluoro)methoxy]-6-fluoro-7-[4-(4-fluorophenyl)piperazin-1-yl]-4-oxo-1-[4-(pyrrolidin-1-ylmethyl)phenyl]1,4-dihydroquinoline-3-carboxylic acid

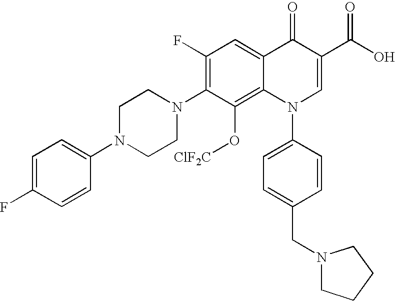

The example was prepared using a similar protocol as Example 146, using 1-(4-fluorophenyl)piperazine in step 1. LC-MS: 645 [M+H]⁺, RT 2.72 min.

### Example 148

### Preparation of 8-[chloro(difluoro)methoxy]-7-[4-(4-chlorophenyl)piperazin-1-yl]-6-fluoro-4-oxo-1-[4-(pyrrolidin-1-ylmethyl)phenyl]-1,4-dihydroquinoline-3-carboxylic acid

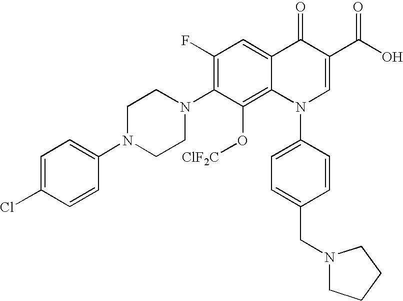

The example was prepared using a similar protocol as Example 146, using 1-(4-chlorophenyl)piperazine in step 1. LC-MS: 661 [M+H]⁺, RT 2.89 min.

### Example 149

### Preparation of 8-[chloro(difluoro)methoxy]-7-[4-(3-cyanopyridin-2-yl)piperazin-1-yl]-6-fluoro-4-oxo-1-[4-(pyrrolidin-1-ylmethyl)-phenyl]-1,4-dihydroquinoline-3-carboxylic acid

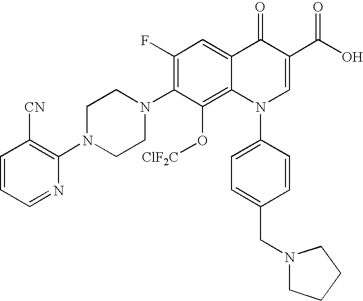

The example was prepared using a similar protocol as Example 146, using 2-piperazin-1-ylnicotinonitrile in step 1. LC-MS: 653 [M+H]⁺, RT 2.53 min.

### Example 150

### Preparation of 8-[chloro(difluoro)methoxy]-7-[4-(2,4-dimethylphenyl)piperazin-1-yl]-6-fluoro-4-oxo-1-[4-(pyrrolidin-1-ylmethyl)phenyl]-1,4-dihydroquinoline-3-carboxylic acid

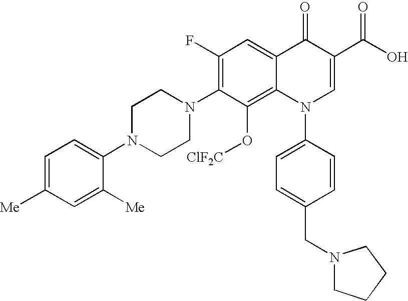

The example was prepared using a similar protocol as Example 146, using 1-(2,4-dimethylphenyl)piperazine in step 1. LC-MS: 655 [M+H]⁺, RT 3.58 min.

### Example 151

### Preparation of 8-[chloro(difluoro)methoxy]-7-[4-(3,4-dimethylphenyl)piperazin-1-yl]-6-fluoro-4-oxo-1-[4-(pyrrolidin-1-ylmethyl)phenyl]-1,4-dihydroquinoline-3-carboxylic acid

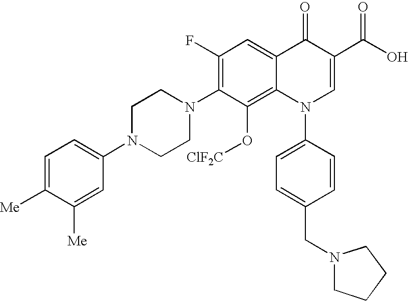

The example was prepared using a similar protocol as Example 146, using 1-(3,4-dimethylphenyl)piperazine in step 1. LC-MS: 655 [M+H]⁺, RT 3.29 min.

### Example 152

### Preparation of 8-[chloro(difluoro)methoxy]-6-fluoro-4-oxo-7-(4-phenylpiperazin-1-yl)-1-[4-(pyrrolidin-1-ylmethyl)phenyl]-1,4-dihydroquinoline-3-carboxylic acid

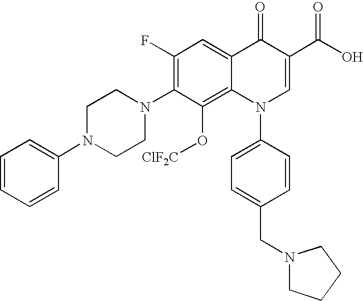

The example was prepared using a similar protocol as Example 146, using 1-phenylpiperazine in step 1. LC-MS: LC-MS: 627 [M+H]⁺, RT 3.25 min.

### Example 153

### Preparation of 6-fluoro-4-oxo-7-(4-pyrimidin-2-yl-piperazin-1-yl)-1-[4-(pyrrolidin-1-ylmethyl)phenyl]-8-(trifluoromethoxy)-1,4-dihydroquinoline-3-carboxylic acid

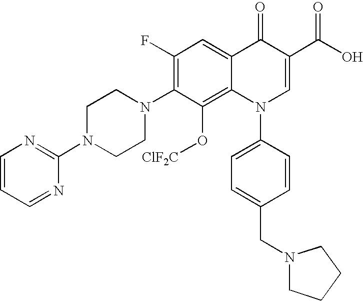

The example was prepared using a similar protocol as Example 146, using 2-piperazin-1-ylpyrimidine in step 1. LC-MS: 613 [M+H]⁺, RT 2.55 min.

### Example 154

### Preparation of 8-[chloro(difluoro)methoxy]-1-{4-[(dimethylamino)methyl]phenyl}-6-fluoro-4-oxo-7-(4-pyridin-2-ylpiperazin-1-yl)-1,4-dihydroquinoline-3-carboxylic acid

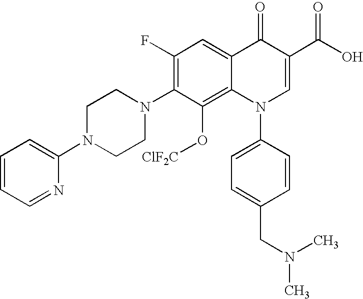

### Step 1: Preparation of 4-[(dimethylamino)methyl]aniline

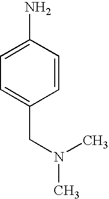

This intermediate was prepared using the procedure as described for preparation of Intermediate A, except dimethyl amine was used instead of pyrrolidine

### Step 2: Preparation of ethyl 8-[chloro(difluoro)methoxy]-1-{4-[(dimethylamino)methyl]phenyl}-6,7-difluoro-4-oxo-1,4-dihydroquinoline-3-carboxylate

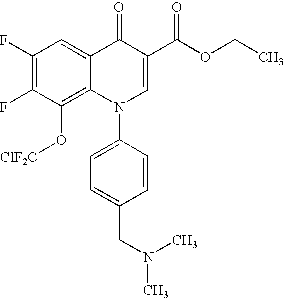

This intermediate was prepared using the procedure as described for preparation of Intermediate D, except dimethyl amine was used instead of 4-pyrrolidin-1-ylmethyl-phenylamine in step 5.

### Step 3: Preparation of the Title Compound

This example was prepared using the procedure as described for preparation of Example 146, except ethyl 8-[chloro(difluoro)methoxy]-1-{4-[(dimethylamino)methyl]phenyl}-6,7-difluoro-4-oxo-1,4-dihydroquinoline-3-carboxylate used instead of Intermediate D in step 1 of the synthesis. LC-MS: 607 [M+H]⁺, RT 2.49 min.

### Example 155

### Preparation of 8-[chloro(difluoro)methoxy]-1-{4-[(dimethylamino)methyl]phenyl}-6-fluoro-7-[4-(4-fluorophenyl)piperazin-1-yl]-4-oxo-1,4-dihydroquinoline-3-carboxylic acid

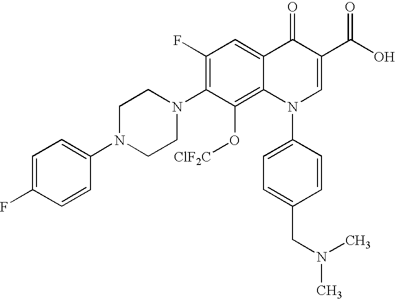

The example was prepared using a similar protocol as Example 154, using 1-(4-fluorophenyl)piperazine instead of 1-pyridin-2-ylpiperazine. LC-MS: 619 [M+H]⁺, RT 2.65 min.

### Example 156

### Preparation of 8-[chloro(difluoro)methoxy]-7-[4-(4-chlorophenyl)piperazin-1-yl]-1-{4-[(dimethylamino)methyl]phenyl}-6-fluoro-4-oxo-1,4-dihydroquinoline-3-carboxylic acid

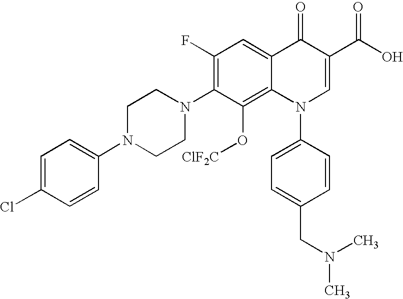

The example was prepared using a similar protocol as Example 154, using 1-(4-chlorophenyl)piperazine instead of 1-pyridin-2-ylpiperazine. LC-MS: 635 [M+H]⁺, RT 2.82 min.

### Example 157

### Preparation of 8-[chloro(difluoro)methoxy]-1-{4-[(dimethylamino)methyl]phenyl}-6-fluoro-4-oxo-7-(4-pyrimidin-2-ylpiperazin-1-yl)-1,4-dihydroquinoline-3-carboxylic acid

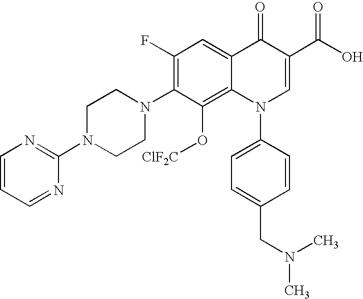

The example was prepared using a similar protocol as Example 154, using 2-piperazin-1-ylpyrimidine instead of 1-pyridin-2-ylpiperazine. LC-MS: 603 [M+H]⁺, RT 2.55 min.

### Example 158

### Preparation of 8-[chloro(difluoro)methoxy]-7-[4-(3-cyanopyridin-2-yl)piperazin-1-yl]-1-{4-[(dimethylamino)methyl]phenyl}-6-fluoro-4-oxo-1,4-dihydroquinoline-3-carboxylic acid

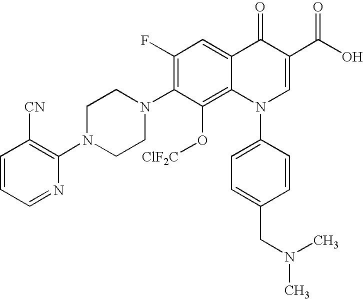

The example was prepared using a similar protocol as Example 154, using 2-piperazin-1-ylnicotinonitrile instead of 1-pyridin-2-ylpiperazine. LC-MS: 627 [M+H]⁺, RT 3.02 min.

### Example 159

### Preparation of 8-[chloro(difluoro)methoxy]-1-{4-[(diethylamino)methyl]phenyl}-6-fluoro-4-oxo-7-(4-pyridin-2-ylpiperazin-1-yl)-1,4-dihydroquinoline-3-carboxylic acid

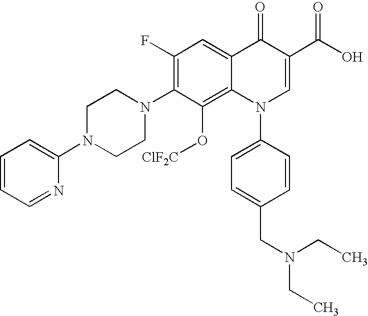

### Step 1: Preparation of 4-[(diethylamino)methyl]aniline

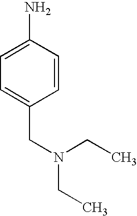

This intermediate was prepared using the procedure as described for preparation of Intermediate A, except diethyl amine was used instead of pyrrolidine

### Step 2: Preparation of ethyl 8-[chloro(difluoro)methoxy]-1-{4-[(diethylamino)methyl]phenyl}-6,7-difluoro-4-oxo-1,4-dihydroquinoline-3-carboxylate

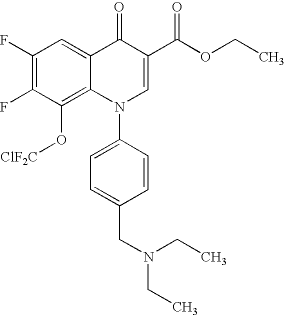

This intermediate was prepared using the procedure as described for preparation of Intermediate D, except 4-[(diethylamino)methyl]aniline was used instead of 4-pyrrolidin-1-ylmethyl-phenylamine in step 5.

Step 3: Preparation of the title compound: This example was prepared using the procedure as described for preparation of Example 147, except ethyl 8-[chloro(difluoro)methoxy]-1-{4-[(diethylamino)methyl]phenyl}-6,7-difluoro-4-oxo-1,4-dihydroquinoline-3-carboxylate was used instead of Intermediate D in step 1 of the synthesis. LC-MS: 630 [M+H]⁺, RT 2.51 min.

### Example 160

### Preparation of 8-[chloro(difluoro)methoxy]-1-{4-[(diethylamino)methyl]phenyl}-6-fluoro-4-oxo-7-(4-pyridin-2-ylpiperazin-1-yl)-1,4-dihydroquinoline-3-carboxylic acid

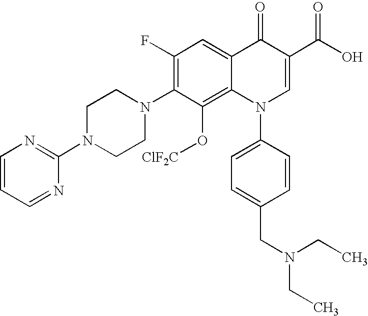

The example was prepared using a similar protocol as Example 159, using 2-piperazin-1-ylpyrimidine instead of 1-pyridin-2-ylpiperazine. LC-MS: 631 [M+H]⁺, RT 2.47 nm in.

### Example 161

### Preparation of 8-[chloro(difluoro)methoxy]-7-[4-(3-cyanopyridin-2-yl)piperazin-1-yl]-1-{4-[(diethylamino)methyl]phenyl}-6-fluoro-4-oxo-1,4-dihydroquinoline-3-carboxylic acid

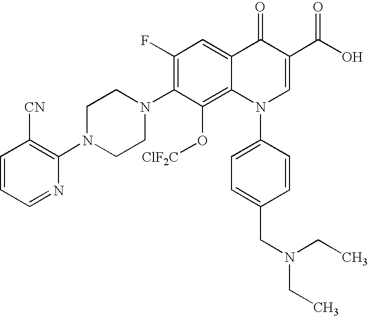

The example was prepared using a similar protocol as Example 159, using 2-piperazin-1-ylnicotinonitrile instead of 1-pyridin-2-ylpiperazine. LC-MS: 655 [M+H]⁺, RT 2.60 min.

### Example 162

### Preparation of 8-[chloro(difluoro)methoxy]-7-[4-(2-cyanophenyl)piperazin-1-yl]-1-{4-[(diethylamino)methyl]phenyl}-6-fluoro-4-oxo-1,4-dihydroquinoline-3-carboxylic acid

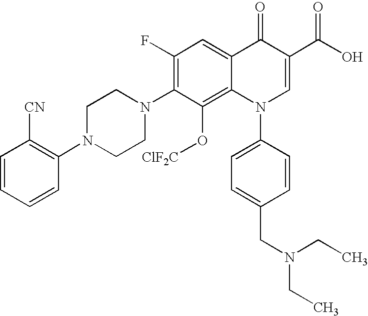

The example was prepared using a similar protocol as Example 159, using 2-piperazin-1-ylbenzonitrile instead of 1-pyridin-2-ylpiperazine. LC-MS: 654 [M+H]⁺, RT 2.73 min.

### Example 163

### Preparation of 8-[chloro(difluoro)methoxy]-1-{4-[(diethylamino)methyl]phenyl}-6-fluoro-7-[4-(4-fluorophenyl)piperazin-1-yl]-4-oxo-1,4-dihydroquinoline-3-carboxylic acid

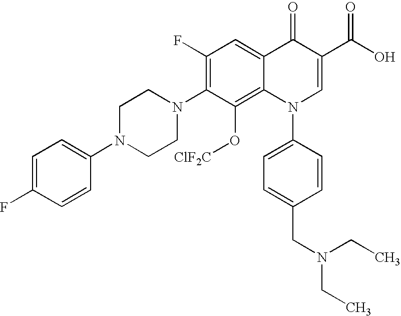

The example was prepared using a similar protocol as Example 159, using 1-(4-fluorophenyl)piperazine instead of 1-pyridin-2-ylpiperazine. LC-MS: 647 [M+H]⁺, RT 2.76 min.

### Example 164

### Preparation of 8-[chloro(difluoro)methoxy]-7-[4-(4-chlorophenyl)piperazin-1-yl]-1-{4-[(diethylamino)methyl]phenyl}-6-fluoro-4-oxo-1,4-dihydroquinoline-3-carboxylic acid

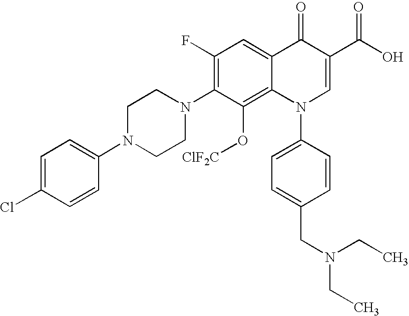

The example was prepared using a similar protocol as Example 159, using 1-(4-chlorophenyl)piperazine instead of 1-pyridin-2-ylpiperazine. LC-MS: 663 [M+H]⁺, RT 2.94 min.

### Example 165

### Preparation of 8-[chloro(difluoro)methoxy]-1-(4-{[ethyl(methyl)amino]methyl}phenyl)-6-fluoro-4-oxo-7-(4-pyrimidin-2-ylpiperazin-1-yl)-1,4-dihydroquinoline-3-carboxylic acid

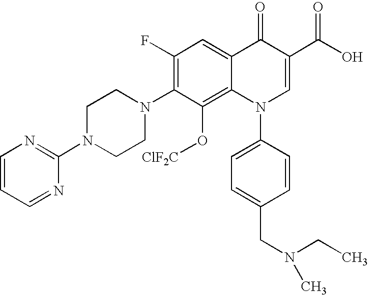

### Step 1: Preparation of 4-{[ethyl(methyl)amino]methyl}aniline

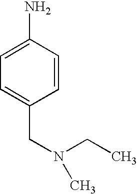

This intermediate was prepared using the procedure as described for preparation of Intermediate A, except N-methyl-N′-ethyl amine was used instead of pyrrolidine

### Step 2: Preparation of ethyl 8-[chloro(difluoro)methoxy]-1-(4-{[ethyl(methyl)amino]methyl}phenyl)-6,7-difluoro-4-oxo-1,4-dihydroquinoline-3-carboxylate

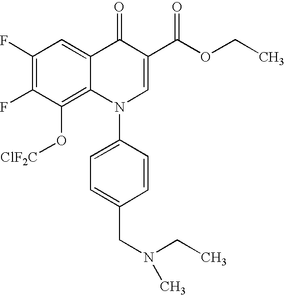

This intermediate was prepared using the procedure as described for preparation of Intermediate D, except 4-{[ethyl(methyl)amino]methyl}aniline was used instead of 4-pyrrolidin-1-ylmethyl-phenylamine in step 5.

### Step 3: Preparation of the Title Compound

This example was prepared using procedure as described for preparation of Example 146, except ethyl 8-[chloro(difluoro)methoxy]-1-(4-{[ethyl(methyl)amino]methyl}phenyl)-6,7-difluoro-4-oxo-1,4-dihydroquinoline-3-carboxylate was used instead of Intermediate D and 2-piperazin-1-ylpyrimidine was used instead of 1-pyridin-2-ylpiperazine in step 1 of the synthesis. LC-MS: 617 [M+H]⁺, RT 2.49 min.

### Example 166

### Preparation of 8-[chloro(difluoro)methoxy]-7-[4-(3-cyanopyridin-2-yl)piperazin-1-yl]-1-(4-{[ethyl(methyl)amino]methyl}phenyl)-6-fluoro-4-oxo-1,4-dihydroquinoline-3-carboxylic acid

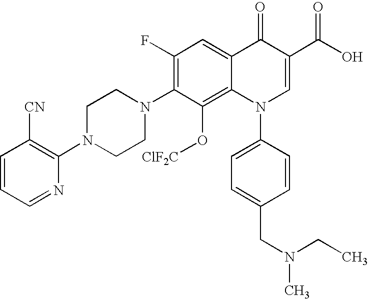

The example was prepared using a similar protocol as Example 164, using 2-piperazin-1-ylnicotinonitrile instead of 2-piperazin-1-ylpyrimidine. LC-MS: 641 [M+H]⁺, RT 2.58 min.

### Example 167

### Preparation of 8-[chloro(difluoro)methoxy]-1-(4-{[ethyl(methyl)amino]methyl}phenyl)-6-fluoro-7-[4-(4-fluorophenyl)piperazin-1-yl]-4-oxo-1,4-dihydroquinoline-3-carboxylic acid

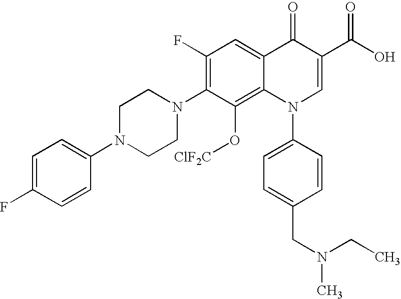

The example was prepared using a similar protocol as Example 164, using 1-(4-fluorophenyl)piperazine instead of 2-piperazin-1-ylpyrimidine. LC-MS: 633 [M+H]⁺, RT 2.76 min.

### Example 168

### Preparation of 8-[chloro(difluoro)methoxy]-7-[4-(4-chlorophenyl)piperazin-1-yl]-1-(4-{[ethyl(methyl)amino]methyl}phenyl)-6-fluoro-4-oxo-1,4-dihydroquinoline-3-carboxylic acid

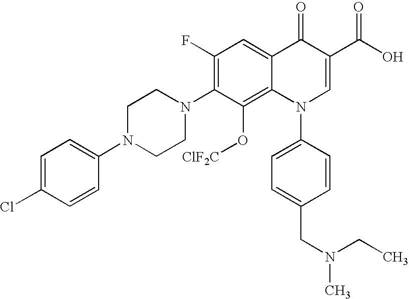

The example was prepared using a similar protocol as Example 164, using 1-(4-chlorophenyl)piperazine instead of 2-piperazin-1-ylpyrimidine. LC-MS: 649 [M+H]⁺, RT 2.92 min.

### Example 169

### Preparation of 8-cyano-6-fluoro-4-oxo-7-(4-pyridin-2-ylpiperazin-1-yl)-1-[4-(pyrrolidin-1-ylmethyl)phenyl]-1,4-dihydroquinoline-3-carboxylic acid

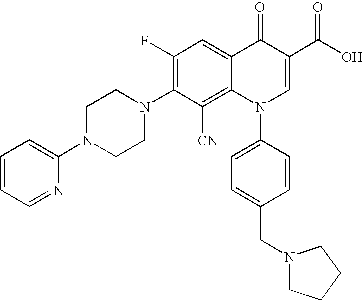

### Step 1: Preparation of ethyl 8-cyano-6-fluoro-4-oxo-7-(4-pyridin-2-ylpiperazin-1-yl)-1-[4-(pyrrolidin-1-ylmethyl)phenyl]-1,4-dihydroquinoline-3-carboxylate

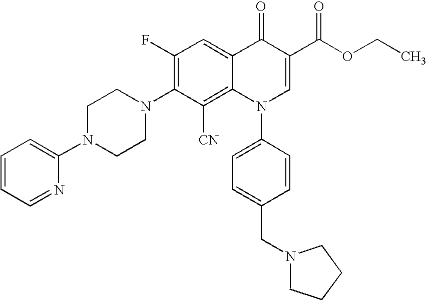

A suspension of [4-(pyrrolidin-1-ylmethyl)phenyl]amine hydrochloride (0.12 g, 0.56 mmol) in dry DMSO was treated with Hunig's base (0.14 g, 1.12 mmol), followed by ethyl (2Z)-2-(3-cyano-2,4,5-trifluorobenzoyl)-3-ethoxyacrylate (0.9 g, 0.55 mmol) and DBU (0.17 g, 1.12 mmol). The mixture was stirred at rt for 2 h. 1-(2-pyridyl)piperazine (0.27 g, 1.65 mmol) and more Hunig's base (0.57 g, 4.4 mmol) were then added to the reaction solution. The reaction was heated at 100° C. overnight. The reaction mixture was cooled down to rt and the solvent was removed. HPLC purification gave the desired product as a yellow powder (70.2 mg, 20%). ¹H NMR (DMSO-d₆: δ 9.97 (broad s, 1H); 8.32 (s, 1H); 8.16 (d, J=12.4, 1H); 8.08 (m, 1H); 7.82 (d, J=8.4, 2H); 7.72 (d, J=8.4, 2H); 7.66 (t, 1H); 6.99 (d, J=8.4, 1H); 6.74 (t, 1H); 4-49 (d, J=5.6, 2H); 4.24 (q, 2H); 3.62 (s, 4H); 3.44 (s, 4H); 3.39 (m, 2H); 3.12 (m, 2H); 2.05 (m, 2H); 1.89 (m, 2H); 1.27 (t, 3H). MS [M+H]⁺: 581.3 m/z. Calcd 580. RT (LC-MS): 1.76 min.

### Step 2: Preparation of the Title Compound

To a solution of ethyl 8-cyano-6-fluoro-4-oxo-7-(4-pyridin-2-ylpiperazin-1-yl)-1-[4-(pyrrolidin-1-ylmethyl)phenyl]-1,4-dihydroquinoline-3-carboxylate (50 mg, 0.08 mmol) in methanol (2 mL) was added 1N NaOH (2 mL). The solution was stirred at rt for 3 h. After the reaction, the reaction was neutralized with 1N HCl, and then extracted with iPrOH/CHCl3 (1:3). The organic layer washed with brine and water, dried, and concentrated. The crude was purified by HPLC to give 8-cyano-6-fluoro-4-oxo-7-(4-pyridin-2-ylpiperazin-1-yl)-1-[4-(pyrrolidin-1-ylmethyl)phenyl]-1,4-dihydroquinoline-3-carboxylic acid as a light yellow powder (32 mg, 51%). ¹H NMR (DMSO-d₆): δ 11.74 (s, broad, 1H); 8-57 (s, 1H); 8.29 (d, J=12.4, 1H); 8.04 (m, 1H); 7.93 (d, J=8.4, 2H); 7.82 (d, J=8.4, 2H); 7.18 (m, 1H); 6.83 (m, 1H); 4.49 (d, J=5.6, 2H); 3.78 (s, 4H); 3.58 (s, 4H); 3.35 (m, 2H); 3.04(m, 2H); 2.00 (m, 2H); 1.92 (m, 2H). MS [M+H]⁺: 553.2 m/z. Calcd 552. RT (LC-MS): 1.71 min.

### Example 170

### Preparation of 8-chloro-1-(4-{[(cyclopropylmethyl)amino]methyl}phenyl)6-fluoro-4-oxo-7-(4-pyridin-2-ylpiperazin-1-yl)-1,4-dihydroquinoline-3-carboxylic acid

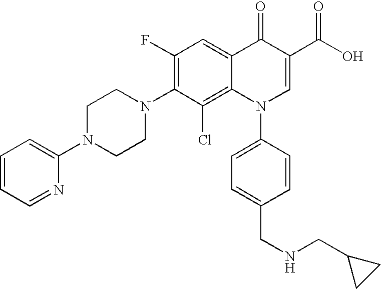

The example was prepared using the procedure as described for the preparation of Example 27. Aminomethylcyclopropane was used in step 2 instead of (3S)-pyrrolidin-3-ol and using 1-pyridin-2-ylpiperazine instead of 2-piperazin-1-ylpyrimidine in step 3. %). ¹H-NMR (DMSO-d₆): [symbol] 9.44 (s, broad, 2H); 8.55 (s, 1H); 8.14 (d, J=12, 1H); 8.03 (m, 1H); 7.91 (m, 1H); 7.76 (d, J=8.4, 2H); 7.69 (d, J=8.4, 2H); 7.30 (m, 1H); 6.91 (m, 1H); 4.26 (m, 2H); 3.78 (m, broad, 4H); 3.37 (s, 4H); 2.86 (m, 2H); 1.16 (m, 1H); 0.62 (m, 2H); 0.41 (m, 2H). MS (M+H)⁺: 562.1 m/z. Calc. 561. RT (LC/MS): 1.39 min.

### Example 171

### Preparation of 8-chloro-1-(4-cyclohexylaminomethyl-phenyl)-6-fluoro-4-oxo-7-(4-pyridin-2-yl-piperazin-1-yl)-1,4-dihydroquinoline-3-carboxylic acid

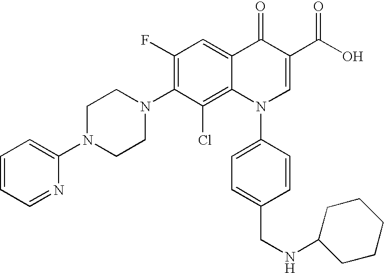

The example was prepared using the procedure as described for the preparation of Example 27. Cyclohexylamine was used in step 2 instead of (3S)-pyrrolidin-3-ol and 1-pyridin-2-ylpiperazine was used instead of 2-piperazin-1-ylpyrimidine in step 3. ¹H-NMR (DMSO-d₆): δ 9.35 (s, broad, 2H); 8.54 (s, 1H); 8.14 (d, J=12, 1H); 8.03 (m, 1H); 7.91 (m, 1H); 7.80 (d, J=8.4, 2H); 7.69 (d, J=8.4, 2H); 7.30 (m, 1H); 6.89 (m, 1H); 4.26 (m, 2H); 3.78 (m, broad, 4H); 3.37 (s, 4H); 2.98 (m, 1H); 2.15 (m, 2H); 1.80 (m, 2H); 1.63 (m, 1H); 1.47 (m, 2H); 1.27 (m, 3H). MS (M+H)⁺: 590.1, (LC/MS): RT 0.98 min.

### Example 172

### Preparation of 8-chloro-6-fluoro-1{4-[(2-hydroxycyclopentylamino)-methyl]-phenyl}-4-oxo-7-(4-pyridin-2-yl-piperazine-1-yl)-1,4-dihydro-quinoline-3-carboxylic acid

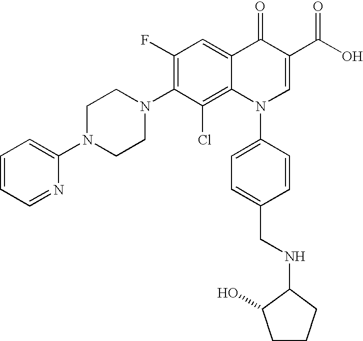

The example was prepared using the procedure as described for the preparation of Example 27. 2-Amino-cyclopentanol was used in step 2 instead of (3S)-pyrrolidin-3-ol and 1-pyridin-2-ylpiperazine was used instead of 2-piperazin-1-ylpyrimidine in step 3. ¹H-NMR (DMSO-d₆): [symbol] 9.70 (broad, 1H); 9.55 (broad, 1H); 8.54 (s, 1H); 8.13 (d, J=12, 1H); 8.02 (m, 1H); 7.93 (m, 1H); 7.80 (d, J=7.6, 2H); 7.69 (d, J=8, 2H); 7-34 (m, 1H); 6.92 (t, 1H); 4.33 (m, 3H); 3.80 (s, broad, 4H); 3.37 (m, 4H); 3.20 (m, 1H); 2-07 (m, 1H); 1.97 (m, 1H); 1.76 (m, 3H); 1.53 (ma, 1H). MS (M+H)⁺: 592.2, (LC/MS): RT 1.41 min.

### Example 173

### Preparation of 8-chloro-1-{4-[(2-chloro-cyclopentylamino)-methyl]-phenyl}-6-fluoro-4-oxo-7-(4-pyridin-2-yl-piperazine-1-yl)-1,4-dihydro-quinoline-3-carboxylic acid

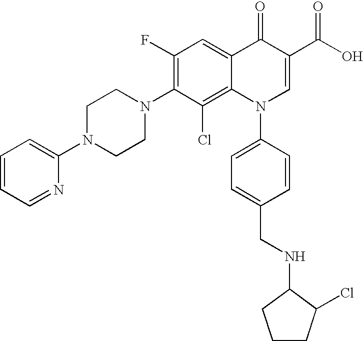

The example was prepared using the procedure as described for the preparation of Example 27. 2-Amino-1-chlorocyclopentane was used in step 2 instead of (3S)-pyrrolidin-3-ol and 1-pyridin-2-ylpiperazine was used instead of 2-piperazin-1-ylpyrimidine in step 3. ¹H-NMR (DMSO-d₆): [symbol] 9.80 (broad, 1H); 9.34 (broad, 1H); 8.53 (s, 1H); 8.14 (d, J=12, 1H); 8.04 (m, 1H); 7.86 (m, 1H); 7.82 (m, 4H); 7.24 (m, 1H); 6.86 (m, 1H); 4.83 (m, 1H); 4.29 (m, 2H); 3.74 (m, 4H); 3.38 (m, 5H); 2.19 (m, 2H); 2.04 (m, 1H); 1.95 (m, 2H); 1.74 (m, 1H). MS (M+H)⁺: 610.1, (LC/MS): RT 2.12 min.

### Example 174

### Preparation of 8-chloro-1-{4-[(2-cyclohexyl-isopropyl-amino)-methyl]-phenyl}-6-fluoro-4-oxo-7-(4-pyridin-2-yl-piperazine-1-yl)-1,4-dihydro-quinoline-3-carboxylic acid

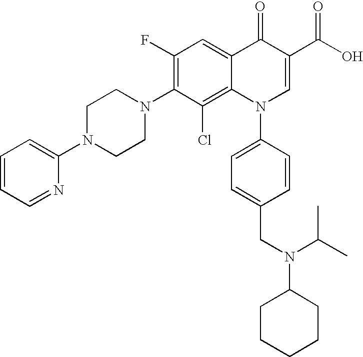

The example was prepared using the procedure as described for the preparation of Example 27. Cyclohexyl-isopropyl amine was used in step 2 instead of (3S)-pyrrolidin-3-ol and 1-pyridin-2-ylpiperazine was used instead of 2-piperazin-1-ylpyrimidine in step 3. ¹H-NMR (DMSO-d₆): [symbol] 9.44 (s, broad, 1H); 8.60 (s, 1H); 8.14 (d, J=11.6, 1H); 8.04 (m, 1H); 7.94 (d, J=8, 1H); 7.87 (m, 1H); 7.84 (d, J=8.4, 1H); 7.72 (m, 2H); 7.27 (m, 1H); 6.88 (m, 1H); 4.53 (m, 2H); 3.76 (m, broad, 4H); 3.14 (m, 5H); 2.22 (m, 2H); 1.80 (m, 2H); 1.61 (m, 3H); 1.43 (d, J=6.8, 3H); 1.38 (d, J=6.4, 3H); 1.29 (m, 3H); 1.13 (m, 1H). MS (M+H)⁺: 632.2, (LC/MS): RT 1.89 min.

### Example 175

### Preparation of 8-chloro-6-fluoro-1-(2-fluoro-4-pyrrolidin-1-ylmethyl-phenyl)-7-[4-(6-methylpyridine-2-yl)-piperazine-1-yl]-4-oxo-1,4-dihydroquinoline-3-carboxylic acid

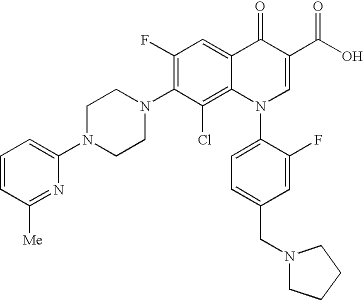

### Step 1: Preparation of 1-(3-fluoro-4-nitrobenzylbromide)

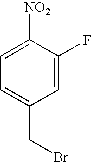

In a 3 L round bottom flask 3-fluoro-4-nitrotoluene (10 g, 64 mmol) was dissolved in methylene chloride (1 L). To the solution, 500 mL of water was added, followed by potassium bromate (43 g, 257 mmol). Sodium hydrosulfite (53 g) in 500 mL of water was added dropwise to the reaction mixture over 10 h. The organic layer was separated, washed and concentrated. The crude product was purified on silica gel column using 0 to 20% EtOAc/hexanes as eluent to furnish 9.7 g (64%) of the desired product.

### Step 2: Preparation of 1-(3-fluoro-4-nitrobenzyl)-pyrrolidine

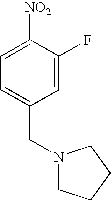

To a solution of 1-(3-fluoro-4-nitrobenzylbromide) (9 g), and disiopropylethylamine (3.6 mL) in THF (80 mL) was added pyrrolidine (2.65 g) in 20 mL of THF at 0° C. The reaction mixture was stirred at rt for 6 h. The resulting precipitate was filtered, and the filtrate was concentrated to give the desired product (2.8 g, 100%) which was used for the next step without further purification. MS (M+H)⁺: 225, (LC/MS): RT 0.91 min.

### Step 3: Preparation of 1-(3-fluoro-4-pyrrolidine-1-yl-methylphenylamine

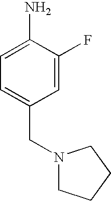

A solution of 1-(3-fluoro-4-nitrobenzyl)-pyrrolidine (2.8 g) was added to the catalyst Raney-Ni in 30 mL of MeOH. The mixture was stirred under an atmosphere of hydrogen (balloon) for 4 h. The reaction mixture was then filtered through a plug of celite, and concentrated to provide the desired product as a light yellow oil (1.5 g, 62%).

### Step 4: Preparation of ethyl 8-chloro-6,7-difluoro-1-(2-fluoro-4-pyrrolidin-1-ylmethylphenyl)-4-oxo-1,4-dihydroquinoline-3-carboxylate

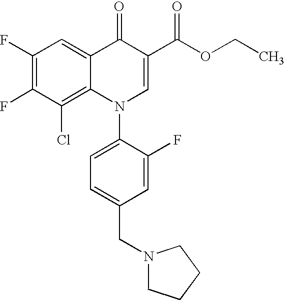

This intermediate was prepared using the procedure as described for preparation of Intermediate B, except 2-fluoro-4-pyrrolidine-1-yl-methylphenylamine was used instead of 4-pyrrolidin-1-ylmethyl-phenylamine.

Step 5: Preparation of the title compound: This example was prepared using the procedure as described for preparation of Example 1, except ethyl 8-chloro-6,7-difluoro-1-(2-fluoro-4-pyrrolidin-1-ylmethylphenyl)-4-oxo-1,4-dihydroquinoline-3-carboxylate was used instead of Intermediate B and 1-(6-methyl-pyridin-2-yl)-piperazine was used instead of 1-(2-pyridylphenyl)piperazine in step 1 of the synthesis. LC-MS: 594 [M+H]⁺, RT 1.92 min.

### Example 176

### Preparation of 8-chloro-6-fluoro-7-[4-(4-fluorophenyl)-piperazine-1-yl]-1-(2-methoxy-4-pyrrolidin-1-ylmethyl-phenyl)-4-oxo-1,4-dihydroquinoline-3-carboxylic acid

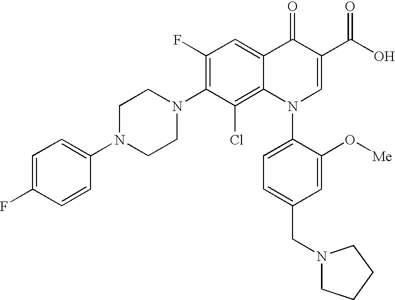

### Step 1: Preparation of 4-amino-3-methoxyphenyl methanol

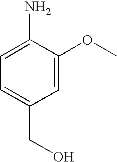

A solution of 3-methoxy-4-nitrobenzyl alcohol (5 g, 27 mmol) in methanol (250 mL) was added to a flask containing 10% Pd—C (5 mol %). The mixture was stirred under hydrogen atmosphere for overnight. The reaction mixture was then filtered through a plug of celite and the filtrate was concentrated. The crude product was purified on silica gel column eluting with EtOAc/hexanes to give pure product as a colorless oil (0.5 g, 15%).

### Step 2: Preparation of ethyl 8-chloro-6,7-difluoro-1-(4-hydroxymethyl-2-methoxyphenyl)-4-oxo-1,4-dihydroquinoline-3-carboxylate

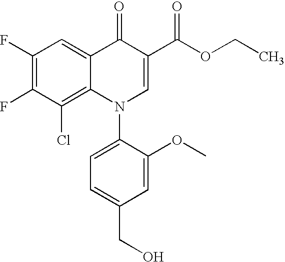

This intermediate was prepared using the procedure as described for preparation of the intermediate F, except that 4-amino-3-methoxyphenyl methanol was used instead of 4-aminobenzyl alcohol in step 1.

Step 3: Preparation of the title compound: This example was prepared using the procedure as described for preparation of Example 27, except that ethyl 8-chloro-6,7-difluoro-1-(4-hydroxymethyl-2-methoxyphenyl)-4-oxo-1,4-dihydroquinoline-3-carboxylate was used instead of Intermediate F in the step 1, pyrrolidine was used in step 2 instead of (3S)-pyrrolidin-3-ol, and 1-(4-fluorophenyl)-piperazine was used instead of 2-piperazin-1-ylpyrimidine in step 3 of the synthesis. LC-MS: 609 [M+H]⁺, RT 2.85 min.

### Example 177

### Preparation of 8-chloro-6-fluoro-7-[4-(4-fluorophenyl)-piperazine-1-yl]-1-(2-methoxy-4-piperidine-1-ylmethyl-phenyl)-4-oxo-1,4-dihydroquinoline-3-carboxylic acid

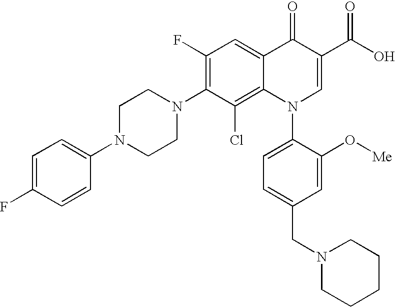

This example was prepared using the procedure as described for preparation of Example 176, except that piperidine was used instead of pyrrolidine in the step 3. LC-MS: 623 [M+H]⁺, RT 2.91 min.

### Example 178

### Preparation of 8-chloro-1-(4-cyclobutylaminomethyl-2-methoxyphenyl)-6-fluoro-7-[4-(4-fluorophenyl)-piperazine-1-yl]-4-oxo-1,4-dihydroquinoline-3-carboxylic acid

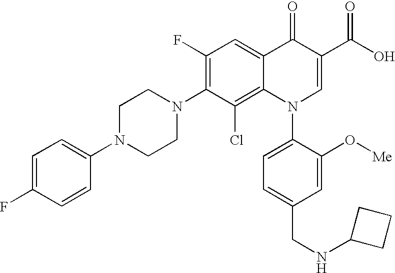

This example was prepared using the procedure as described for preparation of Example 176, except that cyclobutylamine was used instead of pyrrolidine in the step 3. LC-MS: 609 [M+H]⁺, RT 3.19 min.

### Example 179

### Preparation of 8-chloro-1-(4-cyclopentylaminomethyl-2-methoxyphenyl)-6-fluoro-7-[4-(4-fluorophenyl)-piperazine-1-yl]-4-oxo-1,4-dihydroquinoline-3-carboxylic acid

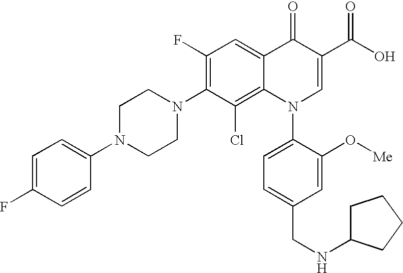

This example was prepared using the procedure as described for preparation of Example 176, except that cyclopentylamine was used instead of pyrrolidine in the step 3. LC-MS: 623 [M+H]⁺, RT 2.91 min.

### Example 180

### Preparation of 8-(chloro-difluoromethoxy)-1-(4-cyclopentylaminomethyl-phenyl)-6-fluoro-4-oxo-7-(4-pyridin-2-yl-piperazin-1-yl)-1,4-dihydroquinoline-3-carboxylic acid

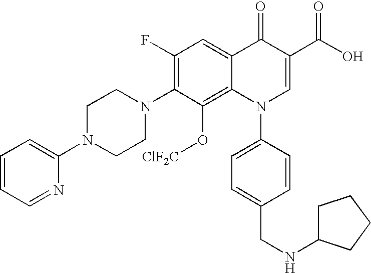

### Step 1: Preparation of ethyl 8-(chloro-difluoromethoxy)-1-(4-cyclopentylaminomethyl-phenyl)-6,7-difluoro-4-oxo-1,4-dihydroquinoline-3-carboxylate

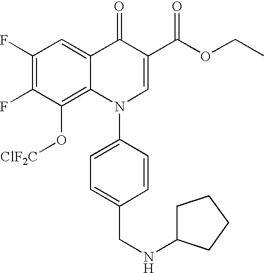

This intermediate was prepared using the procedure as described for preparation of the intermediate D, except that 4-cyclopentylaminomethyl-phenylamine was used instead of 4-pyrrolidine-1 ylmethyl-phenylamine (intermediate A) in step 5.

### Step 2: Preparation of the Title Compound

This example was prepared using procedure as described for preparation of Example 146, except ethyl 8-(chloro-difluoromethoxy)-1-(4-cyclopentylaminomethyl-phenyl)-6,7-difluoro-4-oxo-1,4-dihydroquinoline-3-carboxylate was used instead of Intermediate D. LC-MS: 642 [M+H]⁺, RT 2.71 min.

### Example 181

### Preparation of 8-(chloro-difluoromethoxy)-1-(4-cyclopentylaminomethyl-phenyl)-6-fluoro-4-oxo-7-[4(4-fluorophenyl)piperazine-1-yl]-1,4-dihydroquinoline-3-carboxylic acid

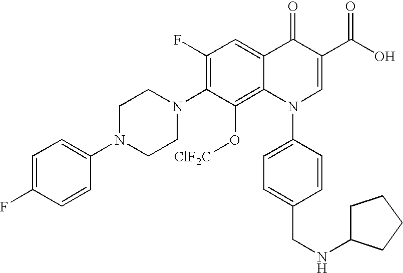

This example was prepared using procedure as described for preparation of Example 180, except that 1-(4-fluorophenyl)piperazine was used in step 2 instead of 1-pyridin-2-ylpiperazine. LC-MS: 659 [M+H]⁺, RT 2.96 min.

### Example 182

### Preparation of 1-(4-cyclopentylaminomethyl-phenyl)-6-fluoro-7-[4-(4-fluorophenyl)-piperazine-1-yl]-4-oxo-8-trifluoromethoxy-1,4-dihydroquinoline-3-carboxylic acid

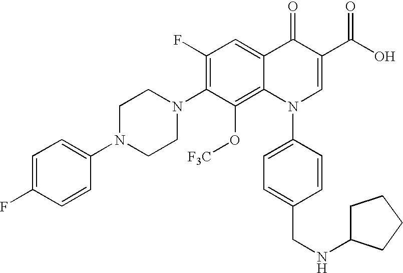

### Step 1: Preparation of 1-(4-cyclopentylaminomethyl-phenyl)-6,7-difluoro-4-oxo-8-trifluoromethoxy-1,4-dihydroquinoline-3-carboxylic acid ethyl ester

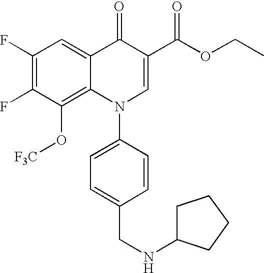

This intermediate was prepared using the procedure as described for preparation of the intermediate E, except 4-cyclopentylaminomethyl-phenylamine was used instead of 4-pyrrolidine-1ylmethyl-phenylamine (intermediate A) in step 3.

### Step 2: Preparation of the Title Compound

This example was prepared using procedure as described for preparation of Example 119, except 1-(4-cyclopentylaminomethyl-phenyl)-6,7-difluoro-4-oxo-8-trifluoromethoxy-1,4-dihydroquinoline-3-carboxylic acid ethyl ester was used instead of Intermediate E and 1-(4-fluorophenyl)piperazine was used instead of 1-(3-chlorophenyl)piperazine in the step 1. LC-MS: 643 [M+H]⁺, RT 2.88 min.

### Example 183

### Preparation of 1-(4-cyclopentylaminomethyl-phenyl)-6-fluoro-7-(4-pyridin-2-yl-piperazine-1-yl]-4-oxo-8-chlorodifluoromethoxy-1,4-dihydroquinoline-3-carboxylic acid

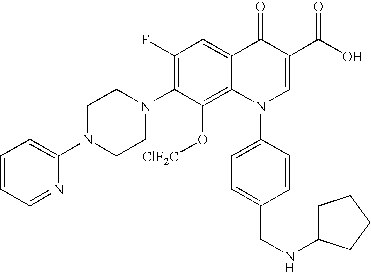

This example was prepared using procedure as described for preparation of Example 182, except that 1-pyridin-2ylpiperazine was used in step 2 instead of 1-(4-fluorophenyl)piperazine. LC-MS: 626 [M+H]⁺, RT 2.77 min.

### Example 184

### Preparation of 8-chloro-7-[4-(3-cyanopyridin-2-yl)-piperazine-1-yl]-6-fluoro-1-(3-fluoro-4-pyrrolidin-1-ylmethyl-phenyl)-4-oxo-1,4-dihydroquinoline-3-carboxylic acid

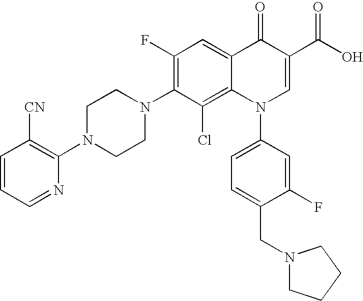

This example was prepared using procedure as described for preparation of Example 175, except that 2-fluoro-4-nitrotoluene was used in step 1, and 2-piperazine-1-yl-nicotinonitrile was used instead of 1-(6-methylpyridin-2-yl)-piperazine in step 5. LC-MS: 605 [M+H]⁺, RT 3.09 min.

### Example 185

### Preparation of 8-chloro-7-[4-(2-cyanophenyl)-piperazine-1-yl]-6-fluoro-1-(3-fluoro-4-pyrrolidin-1-ylmethyl-phenyl)-4-oxo-1,4-dihydroquinoline-3-carboxylic acid

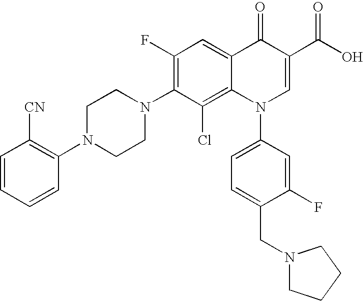

This example was prepared using procedure as described for preparation of Example 184, except 2-piperazine-1-yl-benzonitrile was used instead of 2-piperazine-1-yl-nicotinonitrile in step 5. LC-MS: 604 [M+H]⁺, RT 3.19 min.

### Example 186

### Preparation of 8-chloro-7-[4-(4-fluorophenyl)-piperazine-1-yl]-6-fluoro-1-(3-fluoro-4-pyrrolidin-1-ylmethyl-phenyl)-4-oxo-1,4-dihydroquinoline-3-carboxylic acid

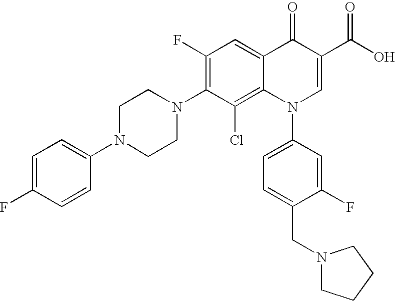

This example was prepared using procedure as described for preparation of Example 184, except 1-(4-fluorophenyl)piperazine was used instead of 2-piperazine-1-yl-nicotinonitrile in step 5. LC-MS: 597 [M+H]⁺, RT 3.11 min.

### Example 187

### Preparation of 8-chloro-7-[4-(4-chlorophenyl)-piperazine-1-yl]-6-fluoro-1-(3-fluoro-4-pyrrolidin-1-ylmethyl-phenyl)-4-oxo-1,4-dihydroquinoline-3-carboxylic acid

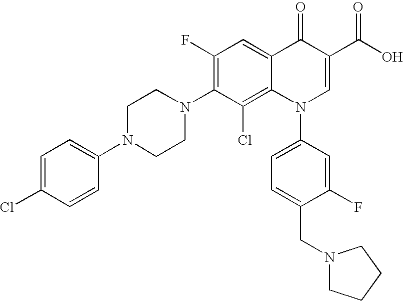

This example was prepared using procedure as described for preparation of Example 1840, except that 1-(4-chlorophenyl)piperazine was used instead of 2-piperazine-1-yl-nicotinonitrile in step 5. LC-MS: 613 [M+H]⁺, RT 3.33 min.

### Example 188

### Preparation of 8-chloro-7-[4-(4-cyanophenyl)-piperazine-1-yl]-6-fluoro-1-(3-fluoro-4-pyrrolidin-1-ylmethyl-phenyl)-4-oxo-1,4-dihydroquinoline-3-carboxylic acid

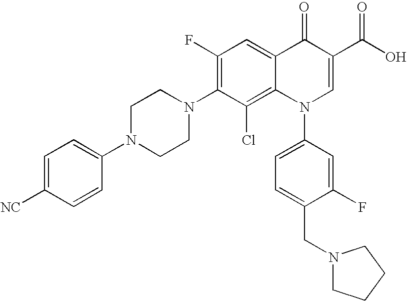

This example was prepared using procedure as described for preparation of Example 184, except that 1-(4-cyanophenyl)piperazine was used instead of 2-piperazine-1-yl-nicotinonitrile in step 5. LC-MS: 604 [M+H]⁺, RT 3.12 min.

### Example 189

### Preparation of 8-chloro-1-(4-cyclohexylaminomethyl-phenyl)-6-fluoro-4-oxo-7-(4-pyrimidin-2-yl-piperazin-1-yl)-1,4-dihydroquinoline-3-carboxylic acid

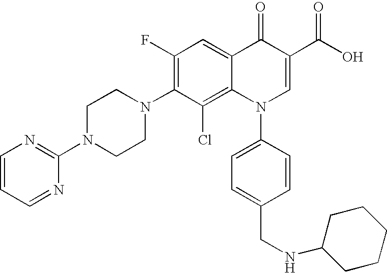

This example was prepared using procedure as described for preparation of Example 56, except that 2-piperazine-1ylpyrimidine was used instead of 1-pyridin-2-ylpiperazine in step 1 and cyclohexylamine was used instead of cyclopentylamine in step 3. LC-MS: 591.2 [M+H]⁺, RT 2.60 min.

### Example 190

### Preparation of 8-chloro-1-{4-[(4-cyclohelmethyl-amino)-methyl]-phenyl}-6-fluoro-4-oxo-7-(4-pyrimidin-2-yl-piperazin-1-yl)-1,4-dihydroquinoline-3-carboxylic acid

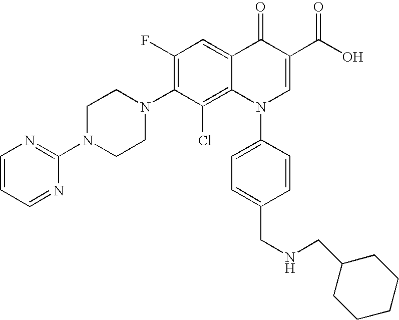

This example was prepared using procedure as described for preparation of Example 56, except that 2-piperazine-1ylpyrimidine was used instead of 1-pyridin-2-ylpiperazine in step 1 and C-cyclohexyl-methylamine was used instead of cyclopentylamine in step 3. LC-MS: 605.2 [M+H]⁺, RT 2.76 min. ¹H NMR (DMSO-d₆): δ 9.02 (s, 1H), 8.53 (s, 1H), 8.34 (d, 2H), 8.10 (d, 1H), 7.33-7.66 (m, 4H), 6.63 (t, 1H), 4.22 (d, 2H), 3.80 (m, 3H), 3.50 (m, 3H), 2.73 (d, 2H), 1.64-1.73 (m, 7H), 1.15-1.18 (m, 3H), 0.80-0.99 (m, 2H).

### Example 191

### Preparation of 8-chloro-1-(4-cyclobutylaminomethyl-phenyl)-6-fluoro-4-oxo-7-(4-pyrimidin-2-yl-piperazin-1-yl)-1,4-dihydroquinoline-3-carboxylic acid

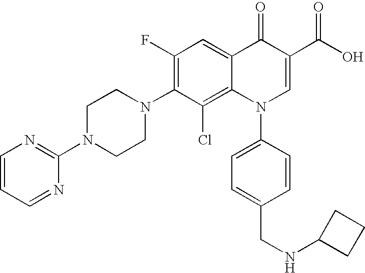

This example was prepared using procedure as described for preparation of Example 56, except that 2-piperazine-1ylpyrimidine was used instead of 1-pyridin-2-ylpiperazine in step 1 and cyclobutylamine was used instead of cyclopentylamine in step 3. LC-MS: 563.2 [M+H]⁺, RT 2.86 min.

### Example 192

### Preparation of 7-[4-(4-cyanophenyl)-piperazine-1-yl]-6-fluoro-8-methoxy-4-oxo-1-(4-piperidin-1-ylmethyl-phenyl)-1,4-dihydroquinoline-3-carboxylic acid

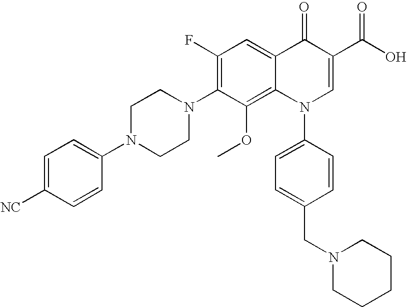

This example was prepared using procedure as described for preparation of Example 89, except that 4-piperazine-1-yl-benzonitrile was used instead of 2-piperazine-1-yl-benzonitrile in step 2. LC-MS: 596.2 [M+H]⁺, RT 3.08 min.

### Example 193

### Preparation of 6-fluoro-8-methoxy-4-oxo-1-(4-piperidin-1-ylmethyl-phenyl)-7-(4-pyrimidine-2-yl-piperazine-1-yl]-1,4-dihydroquinoline-3-carboxylic acid

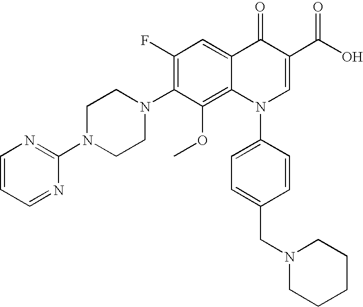

This example was prepared using procedure as described for preparation of Example 89, except that 2-piperazine-1ylpyrimidine was used instead of 2-piperazine-1-yl-benzonitrile in step 2. LC-MS: 573.1 [M+H]⁺, RT 2.38 min. ¹H NMR (DMSO-d₆): δ 10.20 (d, 1H), 8.44 (s, 1H), 8.37 (d, 2H), 7.87 (d, 1H), 7.72 (d, 4H), 6.64 (t, 1H), 4.36 (d, 2H), 3.50-3.84 (m, 5H), 3.32 (d, 2H), 3.17 (s, 3H), 2.86 (d, 2H), 1.69-1.83 (m, 4H), 1.30-1.37 (m, 1H), 1.03 (d, 1H).

### Example 194

### Preparation of 6-fluoro-8-methoxy-4-oxo-1-(4-piperidin-1-ylmethyl-phenyl)-7-(4-pyridin-2-yl-piperazine-1-yl]-1,4-dihydroquinoline-3-carboxylic acid

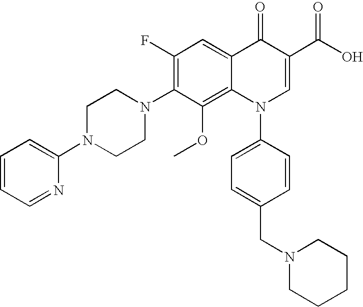

This example was prepared using procedure as described for preparation of Example 89, except that 1-pyridin-2ylpiperazine was used instead of 2-piperazine-1-yl-benzonitrile in step 2. LC-MS: 572.2 [M+H]⁺, RT 1.52 min.

Compositions Useful for the Method of this Invention

A compound of Formula I is useful in this method for preventing or treating the conditions described further herein when it is formulated as a pharmaceutically acceptable composition. A pharmaceutically acceptable composition is a compound of Formula I in admixture with a pharmaceutically acceptable carrier. A pharmaceutically acceptable carrier is any carrier that is relatively non-toxic and innocuous to a patient at concentrations consistent with effective activity of the active ingredient so that any side effects ascribable to the carrier do not vitiate the beneficial effects of the active ingredient.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃);

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate);

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);

chelating agents (examples include but are not limited to edetate disodium and edetic acid);

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate);

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures);

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

The compounds of the present invention can be administered with pharmaceutically-acceptable carriers well known in the art using any effective conventional dosage unit forms formulated as immediate, slow or timed release preparations, including, for example, the following.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatin type containing, for example, surfactants, lubricants, and inert fillers such as lactose, sucrose, calcium phosphate, and corn starch.

A compound used in this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

It is believed that one skilled in the art, utilizing the preceding information, can utilize the present invention to its fullest extent. Nevertheless, the following are examples of pharmaceutical formulations that can be used in the method of the present invention. They are for illustrative purposes only, and are not to be construed as limiting the invention in any way.

Pharmaceutical compositions according to the present invention can be further illustrated as follows:

Sterile IV Solution: A 5 mg/mL solution of the desired compound of this invention is made using sterile, injectable water, and the pH is adjusted if necessary. The solution is diluted for administration to 1-2 mg/mL with sterile 5% dextrose and is administered as an IV infusion over 60 min.

Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 min.

Intramuscular suspension: The following solution or suspension can be prepared, for intramuscular injection:
50 mg/mL of the desired, water-insoluble compound of this invention
   5 mg/mL sodium carboxymethylcellulose
   4 mg/mL TWEEN 80
   9 mg/mL sodium chloride
   9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Method of Treating Cancer

The compounds and compositions described herein can be used to treat or prevent hyper-proliferative disorders. An effective amount of a compound or composition of this invention can be administered to a patient in need thereof in order to achieve a desired pharmacological effect. A patient, for the purpose of this invention, is a mammal, including a human, in need of treatment (including prophylactic treatment) for a particular disorder described further herein. A pharmaceutically effective amount of compound or composition is that amount which produces a desired result or exerts an influence on the particular hyper-proliferative disorder being treated.

Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/-oropharyngeal cancer, and lip and oral cavity cancer,

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

The disorders described above have been well characterized in humans, but also exist with a similar etiology in other mammals. Accordingly, the method of this invention can be administered to mammals, including humans, in need thereof for the treatment of angiogenesis and/or proliferative dependent disorders.

The anti-proliferative activity of the compounds of the method of the present invention can be illustrated, for example, by their activity in vitro in the in vitro tumor cell proliferation assay described below. The link between activity in tumor cell proliferation assays in vitro and anti-tumor activity in the clinical setting has been very well established in the art. For example, the therapeutic utility of taxol (Silvestrini et al. Stem Cells 1993, 11(6), 528-35), taxotere (Bissery et al. Anti Cancer Drugs 1995, 6(3), 339), and topoisomerase inhibitors (Edelman et al. Cancer Chemother. Pharmacol. 1996, 37(5), 385-93) was demonstrated with the use of in vitro tumor proliferation assays.

The compounds and compositions described herein, including salts and esters thereof, exhibit anti-proliferative activity and are thus useful to prevent or treat the disorders associated with hyper-proliferation. The assay described below is one of the methods by which compound activity relating to treatment of the disorders identified herein can be determined.

In Vitro Tumor Cell Proliferation Assay

The adherent H460 human non-small cell lung carcinoma and Colo205 human colon carcinoma cell lines were purchased from the American Type and Culture Collection (ATCC, Manassas, Va.) and maintained in RPMI-1640 growth media supplemented with 10% heat inactivated fetal bovine serum (Gibco, Invitrogen Corp. Grand Island, N.Y.). At 37° C. in a humidified atmosphere of 5% CO₂.

The CellTiter 960 Aqᵤₑₒᵤₛ One Solution kit, MTS, (Promega, Madison, Wis.) was used to measure proliferation of tumor cell lines in vitro. This method monitors the bioreduction of a tetrazolium dye as a measure of cell viability. On Day 0, exponentially growing cells were trypsinized, resuspended in RPMI-1640 growth media supplemented with 10% FCS, 100 u/ml of penicillin G and 100 ug/ml of streptomycin sulfate, and seeded at 2000 cells per well into 96 well microtiter plates. Cells were incubated overnight in a humidified atmosphere of 5% CO₂ at 37° C. On Day 1, serial dilutions of compounds were prepared at 2× the finial assay concentration. One hundred microliters of 2× solution was added to test wells in duplicate and control wells received no test compound. The final drug concentration ranged from 0 to 10-20 um in a 5 point dose-response curve. Cells were incubated in the presence of test compounds in a humidified atmosphere of 5% CO₂ at 37° C. for 72 hours. After 72 hours of compound exposure, 40 ul of Promega CellTiter 960 Aqᵤₑₒᵤₛ One Solution was added to each well and absorbance at 490 n-M was measured using a multi-well plate reader. Percent inhibition of proliferation was calculated using the following formula:
100×(1−Absorbance_{treated}−Background/(Absorbance_{control}−Background)

Where:

Absorbance_{treated}=absorbance at 490 nM in test wells, cells with test compound

Absorbance_{control}=absorbance at 490 nM in control wells, cells with no test compound

Background=absorbance 490 nM in wells containing media and no cells

The concentration of test compound required to inhibit proliferation of 50% of the cells (IC₅₀) was determined by linear regression analysis.

Representative compounds of the invention were tested in the above-described Colo205 human colon carcinoma cell line in vitro assay and found to inhibit human colon carcinoma cell proliferation, and are summarized as follows:

Compounds with IC₅₀ of <500 nM

Example numbers: 1, 2, 4, 6, 7, 13, 15, 17, 18, 19, 21, 23, 24, 25, 26, 27, 28, 29, 30, 31, 32, 35, 37, 38, 39, 40, 41, 42, 43, 44, 45, 48, 56, 57, 58, 59, 60, 61, 62, 69, 72, 73, 74, 77, 79, 85, 88, 89, 90, 92, 93, 96, 97, 98, 102, 105, 106, 108, 110, 113, 114, 115, 116, 117, 118, 119, 120, 121, 122, 123, 124, 125, 126, 127, 128, 130, 131, 132, 133, 135, 136, 137, 138, 139, 146, 147, 148, 149, 151, 152, 153, 154, 155, 156, 158, 159, 161, 162, 163, 164, 165, 166, 167, 168, 170, 171, 172, 174, 175, 176, 177, 178, 180, 181, 182, 183, 184, 185, 186, 187, 188, 189, 191, 192, 193, and 194.

Compounds with IC₅₀ of 500-2,000 nM

Example numbers: 3, 5, 8, 9, 11, 14, 16, 22, 34, 36, 46, 47, 55, 70, 75, 76, 78, 80, 81, 83, 84, 86, 87, 91, 94, 95, 99, 101, 103, 107, 109, 111, 129, 134, 140, 141, 143, 145, 150, 157, 160, 169, 173, and 179.

Compounds with IC₅₀ of 2,000-20,000 nM

Example numbers: 10, 12, 20, 33, 49, 50, 51, 52, 53, 54, 63, 64, 65, 66, 67, 68, 71, 82, 100, 104, 112, 142, 144, and 190.

Based upon the above and other standard laboratory techniques known to evaluate compounds useful for the prevention or treatment of the diseases or disorders described above by standard toxicity tests and by standard pharmacological assays for the determination of the prevention or treatment of the conditions identified above in mammals, and by comparison of these results with the results of known medicaments that are used to treat these conditions, the effective dosage of the compounds of this invention can readily be determined for prevention or treatment of each desired indication. The amount of the active ingredient to be administered in the prevention and/or treatment of one of these conditions can vary widely according to such considerations as the particular compound and dosage unit employed, the mode of administration, the duration of treatment (including prophylactic treatment), the age and sex of the patient treated, and the nature and extent of the condition to be prevented and/or treated.

The total amount of the active ingredient to be administered will generally range from about 0.001 mg/kg to about 300 mg/kg, and preferably from about 0.10 mg/kg to about 150 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of administration and number of doses of a compound or composition of the present invention or a pharmaceutically acceptable salt or ester thereof can be ascertained by those skilled in the art using conventional prevention and/or treatment tests.

The compounds of this invention can be administered as the sole pharmaceutical agent or in combination with one or more other pharmaceutical agents where the combination causes no unacceptable adverse effects. For example, the compounds of this invention can be combined with other anti-hyper-proliferative or other indication agents, and the like, as well as with admixtures and combinations thereof.

For example, optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment and/or prevention of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of this invention include but are not limited to other anti-cancer agents such as epothilone, irinotecan, raloxifen and topotecan.

Other embodiments of the invention will be apparent to the skilled in the art from a consideration of this specification or practice of the invention disclosed herein. It is intended that the specification and examples be considered as exemplary only, with the true scope and spirit of the invention being indicated by the following claims.

1. A compound having the structure (I) 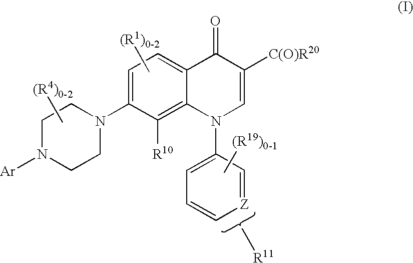
wherein
R¹ represents
—F,
—Cl,
—Br,
—NO₂,
—(C₁₋₃ alkyl) optionally substituted with halogen, or
—NR²R³, wherein
R² and R³ are independently H or C₁₋₃ alkyl optionally substituted with halogen;
R⁴ represents —F, —Cl, —Br, or —(C₁₋₃ alkyl) optionally substituted with halogen;
Ar represents: 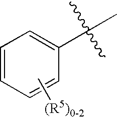
wherein
R⁵ represents
—F,
—Cl,
—Br,
—(C₁₋₃ alkyl) optionally substituted with halogen,
—O(C₁₋₃ alkyl) optionally substituted with halogen,
—S(C₁₋₃ alkyl) optionally substituted with halogen
—CN,
—C(O)NH₂,
—SO₂NH₂,
—C(O)CH₃,
—NO₂; or
—NR⁶R⁷, wherein
R⁶ and R⁷ are independently H or —(C₁₋₃ alkyl) optionally substituted with halogen; 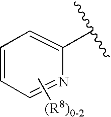
wherein
R⁸ represents
—CN
—(C₁₋₃ alkyl) optionally substituted with halogen,
—O(C₁₋₃ alkyl) optionally substituted with halogen,
—C(O)NH₂, or
—SO₂NH₂; or 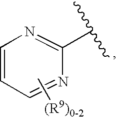
wherein
R⁹ represents —F, —Cl, or —Br;
R¹⁰ represents
—Cl,
—Br,
—(C₁₋₃ alkyl) optionally substituted with halogen,
—O(C₁₋₃ alkyl) optionally substituted with halogen, or
—CN;
Z represents C or N;
when Z is C, R¹¹ is located on one of the two ring atoms encompassed by the bracket and the other of the two ring atoms encompassed by the bracket bears a H or an R¹⁹ substituent, and
when Z is C, R¹¹ represents 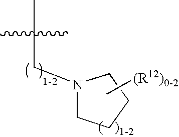
wherein
R¹² represents
—F, —Cl —Br,
—OH,
—(C₁₋₃ alkyl) optionally substituted with halogen,
—O(C₁₋₃ alkyl) optionally substituted with halogen, or —CH₂OR¹³; wherein
R¹³ represents H or —(C₁₋₃ alkyl) optionally substituted with halogen; 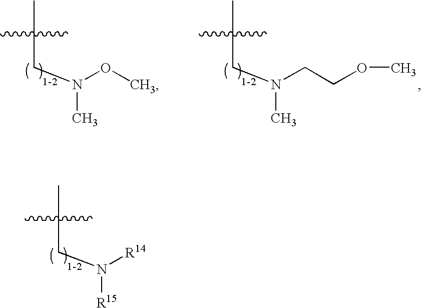
wherein
R¹⁴ represents H or —(C₁₋₃ alkyl) optionally substituted with halogen;
R¹⁵ represents —(CH₂)₀₋₂(C₃₋₆ cycloalkyl) wherein said cycloalkyl moiety is optionally substituted with up to two substituents independently selected from the group of —F, —Cl, —Br, —OH, —(C₁₋₃ alkyl) optionally substituted with halogen, —O(C₁₋₃ alkyl) optionally substituted with halogen, and —CH₂OR¹⁶ wherein
R¹⁶ represents H or —(C₁₋₃ alkyl) optionally substituted with halogen; and 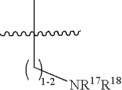
wherein
R¹⁷ represents H or —(C₁₋₃ alkyl) optionally substituted with halogen,
and
R¹⁸ represents —(C₁₋₃ alkyl) optionally substituted with halogen; or 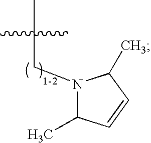
with the proviso that when R¹¹ is —CH₂—NR¹⁷R¹⁸,
R¹⁰ is —Br, —CN, —O(C₁₋₃ alkyl), —OCF₃, —OCF₂Cl, or —(C₁₋₃ alkyl) optionally substituted with halogen;
when Z is N, R¹¹ is located on the carbon atom encompassed by the bracket and R¹¹ represents 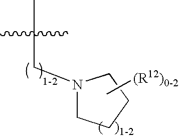
wherein
R¹² is as defined above;
R¹⁹ represents
—F, —Cl, —Br,
—(C₁₋₃ alkyl) optionally substituted with halogen, or
—O(C₁₋₃ alkyl) optionally substituted with halogen;
R²⁰ represents
—NHR²¹, or
—OR²²; wherein
R²¹ and R²² are each independently H or —(C₁₋₃ alkyl) optionally substituted with halogen;
or a pharmaceutically acceptable salt or hydrate thereof.

2. The compound of claim 1 wherein
R¹ represents
—F,
—Br, or
—(C₁₋₃ alkyl) optionally substituted with halogen;
R⁴ is absent;
Ar represents: 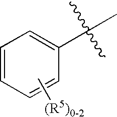
wherein
R⁵ represents
—F,
—Cl,
—Br,
—(C₁₋₃ alkyl) optionally substituted with halogen,
—O(C₁₋₃ alkyl) optionally substituted with halogen,
—S(C₁₋₃ alkyl) optionally substituted with halogen
—CN,
—C(O)NH₂,
—SO₂NH₂,
—C(O)CH₃,
—NO₂; or
—NR⁶R⁷, wherein
R⁶ and R⁷ are independently H or —(C₁₋₃ alkyl) optionally substituted with halogen; 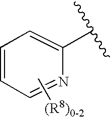
wherein
R⁸ represents
—CN
—(C₁₋₃ alkyl) optionally substituted with halogen,
—O(C₁₋₃ alkyl) optionally substituted with halogen,
—C(O)NH₂, or
—SO₂NH₂; or 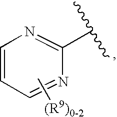
wherein
R⁹ represents —F, —Cl, or —Br;
R¹⁰ represents
—Cl,
—O(C₁₋₃ alkyl) optionally substituted with halogen, or
—CN;
Z represents C;
R¹¹ is located on one of the two ring atoms encompassed by the bracket and the other of the two ring atoms encompassed by the bracket bears a H or an R¹⁹ substituent,
R¹¹ represents 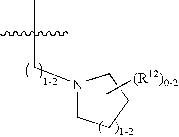
wherein
R¹² represents
—F, —Cl—Br,
—OH,
—(C₁₋₃ alkyl) optionally substituted with halogen,
—O(C₁₋₃ alkyl) optionally substituted with halogen, or
—CH₂OR¹³; wherein
R¹³ represents H or —(C₁₋₃ alkyl) optionally substituted with halogen; 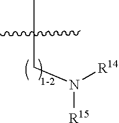
wherein
R¹⁴ represents H or —(C₁₋₃ alkyl) optionally substituted with halogen;
R¹⁵ represents —(CH₂)₀₋₂(C₃₋₆ cycloalkyl) wherein said cycloalkyl moiety is optionally substituted with up to two substituents independently selected from the group of —F, —Cl, —Br, —OH, —(C₁₋₁₃ alkyl) optionally substituted with halogen, —O(C₁₋₃ alkyl) optionally substituted with halogen, and —CH₂OR¹⁶ wherein
R¹⁶ represents H or —(C₁₋₃ alkyl) optionally substituted with halogen; or 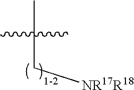
wherein
R¹⁷ represents H or —(C₁₋₃ alkyl) optionally substituted with halogen,
and
R¹⁸ represents —(C₁₋₃ alkyl) optionally substituted with halogen;
with the proviso that when R¹¹ is —CH₂—NR¹⁷R¹⁸,
R¹⁰ is —Br, —CN, —(C₁₋₃ alkyl), —OCF₃, —OCF₂Cl, or —(C₁₋₁₃ alkyl) optionally substituted with halogen;
R¹⁹ represents
—F, —Cl, —Br,
—(C₁₋₃ alkyl) optionally substituted with halogen, or
—O(C₁₋₃ alkyl) optionally substituted with halogen; and
R²⁰ represents
—OR²²; wherein
R²² is H or —(C₁₋₃ alkyl) optionally substituted with halogen.

3. The compound of claim 1 wherein
R¹ is absent or represents a single substituent selected from
—F,
—Cl, and
—Br;
R⁴ is absent;
Ar represents: 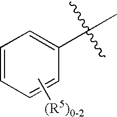
wherein
R⁵ represents
—F,
—Cl,
—Br,
—(C₁₋₃ alkyl) optionally substituted with halogen,
—O(C₁₋₃ alkyl) optionally substituted with halogen, or
—CN; 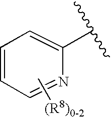
wherein
R⁸ represents
—CN
—(C₁₋₃ alkyl) optionally substituted with halogen, or
—O(C₁₋₃ alkyl) optionally substituted with halogen; or 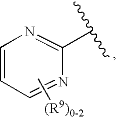
wherein
R⁹ represents —F, —Cl, or —Br;
R¹⁰ represents
—Cl, or
—O(C₁₋₃ alkyl) optionally substituted with halogen;
Z represents C;
R¹¹ is located on one of the two ring atoms encompassed by the bracket and the other of the two ring atoms encompassed by the bracket bears a H or an R¹⁹ substituent,
R¹¹ represents 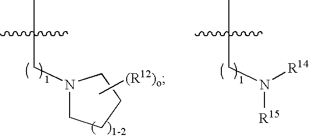
wherein
R¹⁴ represents H or —(C₁₋₃ alkyl) optionally substituted with halogen;
R¹⁵ represents —(CH₂)₀₋₂(C₃₋₆ cycloalkyl) wherein said cycloalkyl moiety is optionally substituted with up to two substituents independently selected from the group of —F, —Cl, —Br, —OH, —(C₁₋₃ alkyl) optionally substituted with halogen, —O(C₁₋₃ alkyl) optionally substituted with halogen, and —CH₂OR¹⁶ wherein
R¹⁶ represents H or —(C₁₋₃ alkyl) optionally substituted with halogen; or 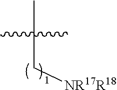
wherein
R¹⁷ represents H or —(C₁₋₃ alkyl) optionally substituted with halogen,
and
R¹⁸ represents —(C₁₋₃ alkyl) optionally substituted with halogen;
with the proviso that when R¹¹ is —CH₂—NR¹⁷R¹⁸,
R¹⁰ is —Br, —CN, —O(C₁₋₃ alkyl), —OCF₃, —OCF₂Cl, or —(C₁₋₃ alkyl) optionally substituted with halogen;
R¹⁹ represents
—F, —Cl, or —Br; and
R²⁰ represents
—OR²²; wherein
R²² is H.

4. A compound selected from the group consisting of:

| Example | |
|---|---|
| Number | Chemical Name |
| 1 | 8-chloro-6-fluoro-4-oxo-7-(4-pyridin-2-yl-piperazin-1-yl)-1-[4- |
| | (pyrrolidin-1-ylmethyl)phenyl]-1,4-dihydroquinoline-3-carboxylic acid |
| 2 | 8-chloro-6-fluoro-7-[4-(4-fluorophenyl)-piperazin-1-yl]-4-oxo-1-[4- |
| | (pyrrolidin-1-ylmethyl)phenyl]-1,4-dihydroquinoline-3-carboxylic acid |
| 4 | 8-chloro-6-fluoro-7-[4-(2-cyanophenyl)-piperazin-1-yl]-4-oxo-1-[4- |
| | (pyrrolidin-1-ylmethyl)phenyl]-1,4-dihydroquinoline-3-carboxylic acid |
| 6 | 8-chloro-6-fluoro-4-oxo-7-(4-pyrimidin-2-ylpiperazin-1-yl)-1-[4- |
| | (pyrrolidin-1-ylmethyl)phenyl]-1,4-dihydroquinoline-3-carboxylic acid |
| 7 | 8-chloro-7-[4-(3-cyanopyridin-2-yl)piperazin-1-yl]-6-fluoro-4-oxo-1- |
| | [4-(pyrrolidin-1-ylmethyl)phenyl]-1,4-dihydroquinoline-3-carboxylic acid |
| 13 | 8-chloro-6-fluoro-7-[4-(6-methylpyridin-2-yl)-piperazin-1-yl]-4-oxo-1- |
| | [4-(pyrrolidin-1-ylmethyl)phenyl]-1,4-dihydroquinoline-3-carboxylic acid |
| 15 | 8-chloro-7-[4-(2-cyanophenyl)piperazin-1-yl]-6-fluoro-4-oxo-1-[4- |
| | (pyrrolidin-1-ylmethyl)phenyl]-1,4-dihydroquinoline-3-carboxylic acid |
| 17 | 8-chloro-6-fluoro-1-{4-[(2-methylpyrrolidin-1-yl)methyl]phenyl}-4- |
| | oxo-7-(4-pyridin-2-ylpiperazin-1-yl)-1,4-dihydroquinoline-3-carboxylic acid |
| 18 | 8-chloro-6-fluoro-7-[4-(4-fluorophenyl)-piperazin-1-yl]-1-{4-[(2- |
| | methylpyrrolidin-1-yl)methyl]phenyl}-4-oxo-1,4-dihydroquinoline-3- |
| | carboxylic acid |
| 19 | 8-chloro-1-{4-[(2,5-dimethylpyrrolidin-1-yl)methyl]phenyl}-6-fluoro- |
| | 4-oxo-7-(4-pyridin-2-ylpiperazin-1-yl)-1,4-dihydro-quinoline-3-carboxylic acid |
| 21 | 8-chloro-6-fluoro-1-(4-{[(2R)-2-(methoxy-methyl)pyrrolidin-1- |
| | yl]methyl}phenyl)-4-oxo-7-(4-pyridin-2-ylpiperazin-1-yl)-1,4- |
| | dihydroquinoline-3-carboxylic acid |
| 23 | 8-chloro-6-fluoro-7-[4-(4-fluorophenyl)-piperazin-1-yl]-1-(4-{[(2R)-2- |
| | (methoxymethyl)pyrrolidin-1-yl]methyl}phenyl)-4-oxo-1,4- |
| | dihydroquinoline-3-carboxylic acid |
| 24 | 8-chloro-6-fluoro-1-(4-{[(2S)-2-(methoxy-methyl)pyrrolidin-1- |
| | yl]methyl}phenyl)-4-oxo-7-(4-pyridin-2-ylpiperazin-1-yl)-1,4- |
| | dihydroquinoline-3-carboxylic acid |
| 25 | 8-chloro-6-fluoro-7-[4-(4-fluorophenyl)-piperazin-1-yl]-1-(4-{[(2S)-2- |
| | (methoxymethyl)pyrrolidin-1-yl]methyl}phenyl)-4-oxo-1,4- |
| | dihydroquinoline-3-carboxylic acid |
| 26 | 8-chloro-6-fluoro-1-(4-{[(2S)-2-(methoxy-methyl)pyrrolidin-1- |
| | yl]methyl}phenyl)-4-oxo-7-(4-pyrimidin-2-ylpiperazin-1-yl)-1,4- |
| | dihydroquinoline-3-carboxylic acid |
| 27 | 8-chloro-6-fluoro-1-(4-{[(3S)-3-hydroxy-pyrrolidin-1- |
| | yl]methyl}phenyl)-4-oxo-7-(4-pyrimidin-2-ylpiperazin-1-yl)-1,4- |
| | dihydroquinoline-3-carboxylic acid |
| 28 | 8-chloro-6-fluoro-1-(4-{[(3S)-3-hydroxy-pyrrolidin-1- |
| | yl]methyl}phenyl)-4-oxo-7-(4-pyridin-2-ylpiperazin-1-yl)-1,4- |
| | dihydroquinoline-3-carboxylic acid |
| 29 | 8-chloro-6-fluoro-7-[4-(4-fluorophenyl)-piperazin-1-yl]-1-(4-{[(3S)-3- |
| | hydroxypyrrolidin-1-yl]methyl}phenyl)-4-oxo-1,4-dihydroquinoline-3- |
| | carboxylic acid |
| 30 | 8-chloro-7-[4-(2-cyanophenyl)piperazin-1-yl]-6-fluoro-1-(4-{[(3S)-3- |
| | hydroxypyrrolidin-1-yl]methyl}phenyl)-4-oxo-1,4-dihydro-quinoline- |
| | 3-carboxylic acid |
| 31 | 8-chloro-6-fluoro-7-[4-(4-fluorophenyl)-piperazin-1-yl]-1-(4-{[(3R)-3- |
| | hydroxypyrrolidin-1-yl]methyl}phenyl)-4-oxo-1,4-dihydroquinoline-3- |
| | carboxylic acid |
| 32 | 8-chloro-7-[4-(2-cyanophenyl)piperazin-1-yl]-6-fluoro-1-(4-{[(3R)-3- |
| | hydroxypyrrolidin-1-yl]methyl}phenyl)-4-oxo-1,4-dihydro-quinoline- |
| | 3-carboxylic acid |
| 35 | 8-chloro-7-[4-(3-cyanopyridin-2-yl)piperazin-1-yl]-6-fluoro-4-oxo-1- |
| | [6-(pyrrolidin-1-ylmethyl)pyridin-3-yl]-1,4-dihydroquinoline-3- |
| | carboxylic acid |
| 37 | 8-chloro-6-fluoro-7-[4-(4-fluorophenyl)-piperazin-1-yl]-4-oxo-1-[6- |
| | (pyrrolidin-1-ylmethyl)pyridin-3-yl]-1,4-dihydroquinoline-3- |
| | carboxylic acid |
| 38 | 8-chloro-7-[4-(4-chlorophenyl)piperazin-1-yl]-6-fluoro-4-oxo-1-[6- |
| | (pyrrolidin-1-ylmethyl)pyridin-3-yl]-1,4-dihydroquinoline-3- |
| | carboxylic acid |
| 39 | 8-chloro-7-[4-(2-cyanophenyl)piperazin-1-yl]-6-fluoro-1-[2-fluoro-4- |
| | (pyrrolidin-1-ylmethyl)phenyl]-4-oxo-1,4-dihydroquinoline-3- |
| | carboxylic acid |
| 40 | 8-chloro-6-fluoro-1-[2-fluoro-4-(pyrrolidin-1-ylmethyl)phenyl]-4-oxo- |
| | 7-(4-pyrimidin-2-ylpiperazin-1-yl)-1,4-dihydroquinoline-3-carboxylic |
| | acid |
| 41 | 8-chloro-6-fluoro-7-[4-(4-fluorophenyl)-piperazin-1-yl]-1-[2-fluoro-4- |
| | (pyrrolidin-1-ylmethyl)phenyl]-4-oxo-1,4-dihydroquinoline-3- |
| | carboxylic acid |
| 42 | 8-chloro-7-[4-(4-chlorophenyl)piperazin-1-yl]-6-fluoro-1-[2-fluoro-4- |
| | (pyrrolidin-1-ylmethyl)phenyl]-4-oxo-1,4-dihydroquinoline-3- |
| | carboxylic acid |
| 43 | 8-chloro-6-fluoro-1-[2-fluoro-4-(pyrrolidin-1-ylmethyl)phenyl]-4-oxo- |
| | 7-(4-phenylpiperazin-1-yl)-1,4-dihydroquinoline-3-carboxylic acid |
| 44 | 8-chloro-6-fluoro-1-[2-fluoro-4-(pyrrolidin-1-ylmethyl)phenyl]-7-[4- |
| | (3-methylpyridin-2-yl)piperazin-1-yl]-4-oxo-1,4-dihydro-quinoline-3- |
| | carboxylic acid |
| 45 | 8-chloro-7-[4-(3-cyanopyridin-2-yl)piperazin-1-yl]-6-fluoro-1-[2- |
| | fluoro-4-(pyrrolidin-1-ylmethyl)phenyl]-4-oxo-1,4-dihydro-quinoline- |
| | 3-carboxylic acid |
| 48 | 8-chloro-7-[4-(4-cyanophenyl)piperazin-1-yl]-6-fluoro-1-[2-fluoro-4- |
| | (pyrrolidin-1-ylmethyl)phenyl]-4-oxo-1,4-dihydroquinoline-3- |
| | carboxylic acid |
| 56 | 8-chloro-1-{4-[(cyclopentylamino)-methyl]phenyl}-6-fluoro-4-oxo-7- |
| | (4-pyridin-2-ylpiperazin-1-yl)-1,4-dihydro-quinoline-3-carboxylic acid |
| 57 | 8-chloro-1-{4-[(cyclopentylamino)methyl]-phenyl}-6-fluoro-4-oxo-7- |
| | (4-pyrimidin-2-ylpiperazin-1-yl)-1,4-dihydroquinoline-3-carboxylic |
| | acid |
| 58 | 8-chloro-1-{4-[(cyclobutylamino)methyl]-phenyl}-6-fluoro-4-oxo-7- |
| | (4-pyridin-2-ylpiperazin-1-yl)-1,4-dihydroquinoline-3-carboxylic acid |
| 59 | 8-chloro-1-{4-[(cyclopropylamino)-methyl]phenyl}-6-fluoro-4-oxo-7- |
| | (4-pyridin-2-ylpiperazin-1-yl)-1,4-dihydro-quinoline-3-carboxylic acid |
| 60 | 8-chloro-1-{4-[(cyclopropylamino)methyl]-phenyl}-6-fluoro-4-oxo-7- |
| | (4-pyrimidin-2-ylpiperazin-1-yl)-1,4-dihydroquinoline-3-carboxylic |
| | acid |
| 61 | 8-chloro-1-(4-{[cyclohexyl(methyl)amino]-methyl}phenyl)-6-fluoro-4- |
| | oxo-7-(4-pyridin-2-ylpiperazin-1-yl)-1,4-dihydro-quinoline-3- |
| | carboxylic acid |
| 62 | 8-chloro-1-(4-{[cyclohexyl(methyl)amino]-methyl}phenyl)-6-fluoro- |
| | 4-oxo-7-(4-pyrimidin-2-ylpiperazin-1-yl)-1,4-dihydro-quinoline-3- |
| | carboxylic acid |
| 69 | 8-chloro-1-{4-[2-(dimethylamino)ethyl]phenyl}-6-fluoro-4-oxo-7-(4- |
| | pyridin-2-ylpiperazin-1-yl)-1,4-dihydroquinoline-3-carboxylic acid |
| 72 | 6-fluoro-8-methoxy-4-oxo-7-(4-pyridin-2-ylpiperazin-1-yl)-1-[4- |
| | (pyrrolidin-1-ylmethyl)phenyl]-1,4-dihydroquinoline-3-carboxylic acid |
| 73 | 6-fluoro-8-methoxy-4-oxo-7-(4-pyrimidin-2-ylpiperazin-1-yl)-1-[4- |
| | (pyrrolidin-1-ylmethyl)phenyl]-1,4-dihydroquinoline-3-carboxylic acid |
| 74 | 6-fluoro-7-[4-(4-fluorophenyl)piperazin-1-yl]-8-methoxy-4-oxo-1-[4- |
| | (pyrrolidin-1-ylmethyl)phenyl]-1,4-dihydroquinoline-3-carboxylic acid |
| 77 | 7-[4-(4-cyanophenyl)piperazin-1-yl]-6-fluoro-8-methoxy-4-oxo-1-[4- |
| | (pyrrolidin-1-ylmethyl)phenyl]-1,4-dihydroquinoline-3-carboxylic acid |
| 79 | 7-[4-(3-cyanopyridin-2-yl)piperazin-1-yl]-6-fluoro-8-methoxy-4-oxo- |
| | 1-[4-(pyrrolidin-1-ylmethyl)phenyl]-1,4-dihydroquinoline-3-carboxylic |
| | acid |
| 85 | 6-fluoro-7-[4-(3-fluorophenyl)piperazin-1-yl]-8-methoxy-4-oxo-1-[4- |
| | (pyrrolidin-1-ylmethyl)phenyl]-1,4-dihydroquinoline-3-carboxylic acid |
| 88 | 6-fluoro-8-methoxy-7-{4-[2-(methylthio)phenyl]-piperazin-1-yl}-4- |
| | oxo-1-[4-(pyrrolidin-1-ylmethyl)phenyl]-1,4-dihydroquinoline-3- |
| | carboxylic acid |
| 89 | 7-[4-(2-cyanophenyl)piperazin-1-yl]-6-fluoro-8-methoxy-4-oxo-1-[4- |
| | (piperidin-1-ylmethyl)phenyl]-1,4-dihydroquinoline-3-carboxylic acid |
| 90 | 7-[4-(4-chlorophenyl)piperazin-1-yl]-6-fluoro-8-methoxy-4-oxo-1-[4- |
| | (piperidin-1-ylmethyl)phenyl]-1,4-dihydroquinoline-3-carboxylic acid |
| 92 | 1-{4-[(dimethylamino)methyl]phenyl}-6-fluoro-8-methoxy-4-oxo-7- |
| | (4-pyridin-2-ylpiperazin-1-yl)-1,4-dihydroquinoline-3-carboxylic acid |
| 93 | 1-{4-[(dimethylamino)methyl]phenyl}-6-fluoro-7-[4-(4- |
| | fluorophenyl)piperazin-1-yl]-8-methoxy-4-oxo-1,4-dihydroquinoline- |
| | 3-carboxylic acid |
| 96 | 1-{4-[(dimethylamino)methyl]phenyl}-6-fluoro-8-methoxy-4-oxo-7- |
| | (4-pyrimidin-2-ylpiperazin-1-yl)-1,4-dihydroquinoline-3-carboxylic |
| | acid |
| 97 | 1-{4-[(dimethylamino)methyl]phenyl}-6-fluoro-8-methoxy-4-oxo-7- |
| | (4-phenylpiperazin-1-yl)-1,4-dihydroquinoline-3-carboxylic acid |
| 98 | 7-[4-(3-chlorophenyl)piperazin-1-yl]-1-{4- |
| | [(dimethylamino)methyl]phenyl}-6-fluoro-8-methoxy-4-oxo-1,4- |
| | dihydroquinoline-3-carboxylic acid |
| 102 | 7-[4-(4-cyanophenyl)piperazin-1-yl]-1-{4- |
| | [(dimethylamino)methyl]phenyl}-6-fluoro-8-methoxy-4-oxo-1,4- |
| | dihydroquinoline-3-carboxylic acid |
| 105 | 1-{4-[(diethylamino)methyl]phenyl}-6-fluoro-7-[4-(4- |
| | fluorophenyl)piperazin-1-yl]-8-methoxy-4-oxo-1,4-dihydroquinoline- |
| | 3-carboxylic acid |
| 106 | 1-{4-[(diethylamino)methyl]phenyl}-6-fluoro-8-methoxy-4-oxo-7-(4- |
| | pyrimidin-2-ylpiperazin-1-yl)-1,4-dihydroquinoline-3-carboxylic acid |
| 108 | 7-[4-(4-cyanophenyl)piperazin-1-yl]-1-{4- |
| | [(diethylamino)methyl]phenyl}-6-fluoro-8-methoxy-4-oxo-1,4- |
| | dihydroquinoline-3-carboxylic acid |
| 110 | 1-{4-[(diethylamino)methyl]phenyl}-6-fluoro-8-methoxy-7-[4-(2- |
| | methoxyphenyl)piperazin-1-yl]-4-oxo-1,4-dihydro-quinoline-3- |
| | carboxylic acid |
| 113 | 7-[4-(3-cyanopyridin-2-yl)piperazin-1-yl]-1-{4- |
| | [(diethylamino)methyl]phenyl}-6-fluoro-8-methoxy-4-oxo-1,4- |
| | dihydroquinoline-3-carboxylic acid |
| 114 | 1-{4-[(diethylamino)methyl]phenyl}-6-fluoro-7-[4-(2- |
| | fluorophenyl)piperazin-1-yl]-8-methoxy-4-oxo-1,4-dihydroquinoline- |
| | 3-carboxylic acid |
| 115 | 7-[4-(3-chlorophenyl)piperazin-1-yl]-1-{4- |
| | [(diethylamino)methyl]phenyl}-6-fluoro-8-methoxy-4-oxo-1,4- |
| | dihydroquinoline-3-carboxylic acid |
| 116 | 7-[4-(4-acetylphenyl)piperazin-1-yl]-1-{4- |
| | [(diethylamino)methyl]phenyl}-6-fluoro-8-methoxy-4-oxo-1,4- |
| | dihydroquinoline-3-carboxylic acid |
| 117 | 7-[4-(2-cyanophenyl)piperazin-1-yl]-1-{4- |
| | [(diethylamino)methyl]phenyl}-6-fluoro-8-methoxy-4-oxo-1,4- |
| | dihydroquinoline-3-carboxylic acid |
| 118 | 7-[4-(4-chlorophenyl)piperazin-1-yl]-1-{4- |
| | [(diethylamino)methyl]phenyl}-6-fluoro-8-methoxy-4-oxo-1,4- |
| | dihydroquinoline-3-carboxylic acid |
| 119 | 7-[4-(3-chlorophenyl)piperazin-1-yl]-6-fluoro-4-oxo-1-[4-(pyrrolidin- |
| | 1-ylmethyl)phenyl]-8-(trifluoromethoxy)-1,4-dihydroquinoline-3- |
| | carboxylic acid |
| 120 | 6-fluoro-7-[4-(6-methylpyridin-2-yl)piperazin-1-yl]-4-oxo-1-[4- |
| | (pyrrolidin-1-ylmethyl)phenyl]-8-(trifluoromethoxy)-1,4- |
| | dihydroquinoline-3-carboxylic acid |
| 121 | 6-fluoro-7-[4-(4-fluorophenyl)piperazin-1-yl]-4-oxo-1-[4-(pyrrolidin- |
| | 1-ylmethyl)phenyl]-8-(trifluoromethoxy)-1,4-dihydroquinoline-3- |
| | carboxylic acid |
| 122 | 6-fluoro-7-[4-(3-methylpyridin-2-yl)-piperazin-1-yl]-4-oxo-1-[4- |
| | (pyrrolidin-1-ylmethyl)phenyl]-8-(trifluoromethoxy)-1,4- |
| | dihydroquinoline-3-carboxylic acid |
| 123 | 6-fluoro-4-oxo-7-(4-pyrimidin-2-yl-piperazin-1-yl)-1-[4-(pyrrolidin-1- |
| | ylmethyl)phenyl]-8-(trifluoromethoxy)-1,4-dihydroquinoline-3- |
| | carboxylic acid |
| 124 | 6-fluoro-4-oxo-7-(4-pyridin-2-yl-piperazin-1-yl)-1-[4-(pyrrolidin-1- |
| | ylmethyl)phenyl]-8-(trifluoromethoxy)-1,4-dihydroquinoline-3- |
| | carboxylic acid |
| 125 | 7-[4-(3-cyanopyridin-2-yl)piperazin-1-yl]-6-fluoro-4-oxo-1-[4- |
| | (pyrrolidin-1-ylmethyl)phenyl]-8-(trifluoromethoxy)-1,4- |
| | dihydroquinoline-3-carboxylic acid |
| 126 | 1-{4-[(dimethylamino)methyl]phenyl}-6-fluoro-4-oxo-7-(4-pyridin-2- |
| | ylpiperazin-1-yl)-8-(trifluoromethoxy)-1,4-dihydro-quinoline-3- |
| | carboxylic acid |
| 127 | 1-{4-[(dimethylamino)methyl]phenyl}-6-fluoro-7-[4-(6-methylpyridin- |
| | 2-yl)piperazin-1-yl]-4-oxo-8(trifluoromethoxy)-1,4-dihydroquinoline- |
| | 3-carboxylic acid |
| 128 | 1-{4-[(dimethylamino)methyl]phenyl}-6-fluoro-4-oxo-7-(4-pyrimidin- |
| | 2-ylpiperazin-1-yl)-8-(trifluoromethoxy)-1,4-dihydro-quinoline-3- |
| | carboxylic acid |
| 130 | 7-[4-(3-chlorophenyl)piperazin-1-yl]-1-{4- |
| | [(dimethylamino)methyl]phenyl}-6-fluoro-4-oxo-8-(trifluoromethoxy)- |
| | 1,4-dihydroquinoline-3-carboxylic acid |
| 131 | 1-{4-[(dimethylamino)methyl]phenyl}-6-fluoro-7-[4-(4- |
| | fluorophenyl)piperazin-1-yl]-4-oxo-8-(trifluoromethoxy)-1,4-dihydro- |
| | quinoline-3-carboxylic acid |
| 132 | 1-{4-[(diethylamino)methyl]phenyl}-6-fluoro-4-oxo-7-(4-pyridin-2- |
| | ylpiperazin-1-yl)-8-(trifluoromethoxy)-1,4-dihydro-quinoline-3- |
| | carboxylic acid |
| 133 | ethyl 1-{4-[(diethylamino)methyl]-phenyl}-6-fluoro-4-oxo-7-(4- |
| | pyrimidin-2-ylpiperazin-1-yl)-8-(trifluoromethoxy)-1,4- |
| | dihydroquinoline-3-carboxylate |
| 135 | 1-{4-[(diethylamino)methyl]phenyl}-6-fluoro-7-[4-(2- |
| | fluorophenyl)piperazin-1-yl]-4-oxo-8-(trifluoromethoxy)-1,4-dihydro- |
| | quinoline-3-carboxylic acid |
| 136 | 7-[4-(3-chlorophenyl)piperazin-1-yl]-1-{4- |
| | [(diethylamino)methyl]phenyl}-6-fluoro-4-oxo-8-(trifluoromethoxy)- |
| | 1,4-dihydro-quinoline-3-carboxylic acid |
| 137 | 1-{4-[(diethylamino)methyl]phenyl}-7-[4-(2,4- |
| | difluorophenyl)piperazin-1-yl]-6-fluoro-4-oxo-8-(trifluoromethoxy)- |
| | 1,4-dihydro-quinoline-3-carboxylic acid |
| 138 | 1-{4-[(diethylamino)methyl]phenyl}-6-fluoro-4-oxo-7-(4- |
| | phenylpiperazin-1-yl)-8-(trifluoromethoxy)-1,4-dihydroquinoline-3- |
| | carboxylic acid |
| 139 | 6-fluoro-1-(4-{[(2S)-2-methylpiperidin-1-yl]methyl}phenyl)-4-oxo-7- |
| | (4-pyrimidin-2-ylpiperazin-1-yl)-8-(trifluoromethoxy)-1,4- |
| | dihydroquinoline-3-carboxylic acid |
| 146 | 8-[chloro(difluoro)methoxy]-6-fluoro-4-oxo-7-(4-pyridin-2- |
| | ylpiperazin-1-yl)-1-[4-(pyrrolidin-1-ylmethyl)phenyl]-1,4- |
| | dihydroquinoline-3-carboxylic acid |
| 147 | 8-[chloro(difluoro)methoxy]-6-fluoro-7-[4-(4-fluorophenyl)piperazin- |
| | 1-yl]-4-oxo-1-[4-(pyrrolidin-1-ylmethyl)phenyl]-1,4-dihydroquinoline- |
| | 3-carboxylic acid |
| 148 | 8-[chloro(difluoro)methoxy]-7-[4-(4-chlorophenyl)piperazin-1-yl]-6- |
| | fluoro-4-oxo-1-[4-(pyrrolidin-1-ylmethyl)phenyl]-1,4- |
| | dihydroquinoline-3-carboxylic acid |
| 149 | 8-[chloro(difluoro)methoxy]-7-[4-(3-cyanopyridin-2-yl)piperazin-1- |
| | yl]-6-fluoro-4-oxo-1-[4-(pyrrolidin-1-ylmethyl)-phenyl]-1,4- |
| | dihydroquinoline-3-carboxylic acid |
| 151 | 8-[chloro(difluoro)methoxy]-7-[4-(3,4-dimethylphenyl)piperazin-1-yl]- |
| | 6-fluoro-4-oxo-1-[4-(pyrrolidin-1-ylmethyl)phenyl]-1,4- |
| | dihydroquinoline-3-carboxylic acid |
| 152 | 8-[chloro(difluoro)methoxy]-6-fluoro-4-oxo-7-(4-phenylpiperazin-1- |
| | yl)-1-[4-(pyrrolidin-1-ylmethyl)phenyl]-1,4-dihydro-quinoline-3- |
| | carboxylic acid |
| 153 | 6-fluoro-4-oxo-7-(4-pyrimidin-2-yl-piperazin-1-yl)-1-[4-(pyrrolidin-1- |
| | ylmethyl)phenyl]-8-(trifluoromethoxy)-1,4-dihydroquinoline-3- |
| | carboxylic acid |
| 154 | 8-[chloro(difluoro)methoxy]-1-{4-[(dimethylamino)methyl]phenyl}-6- |
| | fluoro-4-oxo-7-(4-pyridin-2-ylpiperazin-1-yl)-1,4-dihydroquinoline-3- |
| | carboxylic acid |
| 155 | 8-[chloro(difluoro)methoxy]-1-{4-[(dimethylamino)methyl]phenyl}-6- |
| | fluoro-7-[4-(4-fluorophenyl)piperazin-1-yl]-4-oxo-1,4- |
| | dihydroquinoline-3-carboxylic acid |
| 156 | 8-[chloro(difluoro)methoxy]-7-[4-(4-chlorophenyl)piperazin-1-yl]-1- |
| | {4-[(dimethylamino)methyl]phenyl}-6-fluoro-4-oxo-1,4- |
| | dihydroquinoline-3-carboxylic acid |
| 158 | 8-[chloro(difluoro)methoxy]-7-[4-(3-cyanopyridin-2-yl)piperazin-1- |
| | yl]-1-{4-[(dimethylamino)methyl]phenyl}-6-fluoro-4-oxo-1,4- |
| | dihydroquinoline-3-carboxylic acid |
| 159 | 8-[chloro(difluoro)methoxy]-1-{4-[(diethylamino)methyl]phenyl}-6- |
| | fluoro-4-oxo-7-(4-pyridin-2-ylpiperazin-1-yl)-1,4-dihydroquinoline-3- |
| | carboxylic acid |
| 161 | 8-[chloro(difluoro)methoxy]-7-[4-(3-cyanopyridin-2-yl)piperazin-1- |
| | yl]-1-{4-[(diethylamino)methyl]phenyl}-6-fluoro-4-oxo-1,4- |
| | dihydroquinoline-3-carboxylic acid |
| 162 | 8-[chloro(difluoro)methoxy]-7-[4-(2-cyanophenyl)piperazin-1-yl]-1- |
| | {4-[(diethylamino)methyl]phenyl}-6-fluoro-4-oxo-1,4- |
| | dihydroquinoline-3-carboxylic acid |
| 163 | 8-[chloro(difluoro)methoxy]-1-{4-[(diethylamino)methyl]phenyl}-6- |
| | fluoro-7-[4-(4-fluorophenyl)piperazin-1-yl]-4-oxo-1,4- |
| | dihydroquinoline-3-carboxylic acid |
| 164 | 8-[chloro(difluoro)methoxy]-7-[4-(4-chlorophenyl)piperazin-1-yl]-1- |
| | {4-[(diethylamino)methyl]phenyl}-6-fluoro-4-oxo-1,4- |
| | dihydroquinoline-3-carboxylic acid |
| 165 | 8-[chloro(difluoro)methoxy]-1-(4- |
| | {[ethyl(methyl)amino]methyl}phenyl)-6-fluoro-4-oxo-7-(4-pyrimidin- |
| | 2-ylpiperazin-1-yl)-1,4-dihydroquinoline-3-carboxylic acid |
| 166 | 8-[chloro(difluoro)methoxy]-7-[4-(3-cyanopyridin-2-yl)piperazin-1- |
| | yl]-1-(4-{[ethyl(methyl)amino]methyl}phenyl)-6-fluoro-4-oxo-1,4- |
| | dihydroquinoline-3-carboxylic acid |
| 167 | 8-[chloro(difluoro)methoxy]-1-(4- |
| | {[ethyl(methyl)amino]methyl}phenyl)-6-fluoro-7-[4-(4- |
| | fluorophenyl)piperazin-1-yl]-4-oxo-1,4-dihydroquinoline-3-carboxylic |
| | acid |
| 168 | 8-[chloro(difluoro)methoxy]-7-[4-(4-chlorophenyl)piperazin-1-yl]-1- |
| | (4-{[ethyl(methyl)amino]methyl}phenyl)-6-fluoro-4-oxo-1,4- |
| | dihydroquinoline-3-carboxylic acid |
| 170 | 8-chloro-1-(4-{[(cyclopropylmethyl)amino]methyl}phenyl)6-fluoro-4- |
| | oxo-7-(4-pyridin-2-ylpiperazin-1-yl)-1,4-dihydroquinoline-3- |
| | carboxylic acid |
| 171 | 8-chloro-1-(4-cyclohexylaminomethyl-phenyl)-6-fluoro-4-oxo-7-(4- |
| | pyridin-2-yl-piperazin-1-yl)-1,4-dihydroquinoline-3-carboxylic acid |
| 172 | 8-chloro-6-fluoro-1{4-[(2-hydroxycyclopentylamino)-methyl]- |
| | phenyl}-4-oxo-7-(4-pyridin-2-yl-piperazine-1-yl)-1,4-dihydro- |
| | quinoline-3-carboxylic acid |
| 174 | 8-chloro-1-{4-[(2-cyclohexyl-isopropyl-amino)-methyl]-phenyl}-6- |
| | fluoro-4-oxo-7-(4-pyridin-2-yl-piperazine-1-yl)-1,4-dihydro-quinoline- |
| | 3-carboxylic acid |
| 175 | 8-chloro-6-fluoro-1-(2-fluoro-4-pyrrolidin-1-ylmethyl-phenyl)-7-[4-(6- |
| | methylpyridine-2-yl)-piperazine-1-yl]-4-oxo-1,4-dihydroquinoline-3- |
| | carboxylic acid |
| 176 | 8-chloro-6-fluoro-7-[4-(4-fluorophenyl)-piperazine-1-yl]-1-(2- |
| | methoxy-4-pyrrolidin-1-ylmethyl-phenyl)-4-oxo-1,4-dihydroquinoline- |
| | 3-carboxylic acid |
| 177 | 8-chloro-6-fluoro-7-[4-(4-fluorophenyl)-piperazine-1-yl]-1-(2- |
| | methoxy-4-piperidine-1-ylmethyl-phenyl)-4-oxo-1,4-dihydroquinoline- |
| | 3-carboxylic acid |
| 178 | 8-chloro-1-(4-cyclobutylaminomethyl-2-methoxyphenyl)-6-fluoro-7- |
| | [4-(4-fluorophenyl)-piperazine-1-yl]-4-oxo-1,4-dihydroquinoline-3- |
| | carboxylic acid |
| 180 | 8-(chloro-difluoromethoxy)-1-(4-cyclopentylaminomethyl-phenyl)-6- |
| | fluoro-4-oxo-7-(4-pyridin-2-yl-piperazin-1-yl)-1,4-dihyroquinoline-3- |
| | carboxylic acid |
| 181 | 8-(chloro-difluoromethoxy)-1-(4-cyclopentylaminomethyl-phenyl)-6- |
| | fluoro-4-oxo-7-[4(4-fluorophenyl)piperazine-1-yl]-1,4- |
| | dihyroquinoline-3-carboxylic acid |
| 182 | 1-(4-cyclopentylaminomethyl-phenyl)-6-fluoro-7-[4-(4-fluorophenyl)- |
| | piperazine-1-yl]-4-oxo-8-trifluoromethoxy-1,4-dihyroquinoline-3- |
| | carboxylic acid |
| 183 | 1-(4-cyclopentylaminomethyl-phenyl)-6-fluoro-7-(4-pyridin-2-yl- |
| | piperazine-1-yl]-4-oxo-8-chlorodifluoromethoxy-1,4-dihyroquinoline- |
| | 3-carboxylic acid |
| 184 | 8-chloro-7-[4-(3-cyanopyridin-2-yl)-piperazine-1-yl]-6-fluoro-1-(3- |
| | fluoro-4-pyrrolidin-1-ylmethyl-phenyl)-4-oxo-1,4-dihydroquinoline-3- |
| | carboxylic acid |
| 185 | 8-chloro-7-[4-(2-cyanophenyl)-piperazine-1-yl]-6-fluoro-1-(3-fluoro-4- |
| | pyrrolidin-1-ylmethyl-phenyl)-4-oxo-1,4-dihydroquinoline-3- |
| | carboxylic acid |
| 186 | 8-chloro-7-[4-(4-fluorophenyl)-piperazine-1-yl]-6-fluoro-1-(3-fluoro-4- |
| | pyrrolidin-1-ylmethyl-phenyl)-4-oxo-1,4-dihydroquinoline-3- |
| | carboxylic acid |
| 187 | 8-chloro-7-[4-(4-chlorophenyl)-piperazine-1-yl]-6-fluoro-1-(3-fluoro- |
| | 4-pyrrolidin-1-ylmethyl-phenyl)-4-oxo-1,4-dihydroquinoline-3- |
| | carboxylic acid |
| 188 | 8-chloro-7-[4-(4-cyanophenyl)-piperazine-1-yl]-6-fluoro-1-(3-fluoro-4- |
| | pyrrolidin-1-ylmethyl-phenyl)-4-oxo-1,4-dihydroquinoline-3- |
| | carboxylic acid |
| 189 | 8-chloro-1-(4-cyclohexylaminomethyl-phenyl)-6-fluoro-4-oxo-7-(4- |
| | pyrimidin-2-yl-piperazin-1-yl)-1,4-dihydroquinoline-3-carboxylic acid |
| 191 | 8-chloro-1-(4-cyclobutylaminomethyl-phenyl)-6-fluoro-4-oxo-7-(4- |
| | pyrimidin-2-yl-piperazin-1-yl)-1,4-dihydroquinoline-3-carboxylic acid |
| 192 | 7-[4-(4-cyanophenyl)-piperazine-1-yl]-6-fluoro-8-methoxy-4-oxo-1-(4- |
| | piperidin-1-ylmethyl-phenyl)-1,4-dihydroquinoline-3-carboxylic acid |
| 193 | 6-fluoro-8-methoxy-4-oxo-1-(4-piperidin-1-ylmethyl-phenyl)-7-(4- |
| | pyrimidine-2-yl-piperazine-1-yl]-1,4-dihydroquinoline-3-carboxylic |
| | acid |
| 194 | 6-fluoro-8-methoxy-4-oxo-1-(4-piperidin-1-ylmethyl-phenyl)-7-(4- |
| | pyridin-2-yl-piperazine-1-yl]-1,4-dihydroquinoline-3-carboxylic acid |

5. A pharmaceutical composition comprising a compound of claim 1 and a pharmaceutically acceptable excipient.

6. A method of treating a hyperproliferative disorder comprising administering to a mammalian subject an effective amount of a compound of claim 1.
